(19) United States
(12) Patent Application Publication
(10) Pub. No.: US 2006/0035940 A1
(43) Pub. Date: Feb. 16, 2006

(54) (S)-Amlodipine malate

(75) Inventors: Sharon M. Laughlin, Hudson, MA (US); Roger Bakale, Shrewsbury, MA (US); Harold Scott Wilkinson, Westborough, MA (US); Andrei Zlota, Sharon, MA (US)

Correspondence Address: FOLEY HOAG, LLP;PATENT GROUP, WORLD TRADE CENTER WEST, 155 SEAPORT BLVD, BOSTON, MA, 02110, US

(73) Assignee: Sepracor Inc., Marlborough, MA (US)

(21) Appl. No.: 11/082,253

(22) Filed: Mar. 16, 2005

Related U.S. Application Data
(60) Provisional application No. 60/554,030, filed on Mar. 16, 2004.
(60) Provisional application No. 60/649,635, filed on Feb. 3, 2005.

(51) Int. Cl. C07D 211/82; A61K 31/455

(52) U.S. Cl. 514/356; 546/315

(57) ABSTRACT

One aspect of the present invention relates to optically pure (S)-amlodipine malate. Another aspect of the present invention relates to (rac)-amlodipine malate. In a preferred embodiment, the compound is optically pure (S)-amlodipine L-malate. Another aspect of the present invention relates to a pharmaceutical composition comprising optically pure (S)-amlodipine malate. Another aspect of the present invention relates to a method of preparing optically pure (S)-amlodipine malate, comprising admixing optically pure (S)-amlodipine with malic acid. Another aspect of the present invention relates to the various polymorphic and solvated forms of optically pure (S)-amlodipine malate. In another prefered embodiment the invention relates to polymorphic and solvated forms A-G. The present invention also relates to a method of preparing optically pure (S)-amlodipine malate, comprising combining a salt of optically pure (S)-amlodipine with a malate salt to give optically pure (S)-amlodipine malate. In a preferred embodiment, the malate salt is an optically pure L-malate salt.

### RELATED APPLICATIONS

This application claims the benefit of priority to U.S. Provisional Patent Application Ser. No. 60/554,030, filed Mar. 16, 2004; U.S. Provisional Patent Application Ser. No. 60/649,635, filed Feb. 3, 2005; the contents of both of which are hereby incorporated by reference.

### BACKGROUND OF THE INVENTION

Amlodipine is a dihydropyridine calcium antagonist that inhibits the transmembrane influx of calcium ions into vascular smooth muscle and cardiac muscle. Experimental data suggest that amlodipine binds to both dihydropyridine and non-dihydropyridine binding sites. The contractile processes of cardiac muscle and vascular smooth muscle are dependent upon the movement of extracellular calcium ions into these cells through specific ion channels. The ability of amlodipine to block calcium channels in smooth muscle produces peripheral vasodilation resulting in decreases in both systolic and diastolic blood pressure. Within the physiologic pH range, amlodipine is an ionized compound (pKa=8.6, as reported in Norvasc® package insert), and its kinetic interaction with the calcium channel receptor is characterized by a gradual rate of association and dissociation with the receptor binding site, resulting in a gradual onset of effect. The metabolites of amlodipine do not possess significant calcium-channel blocking activity, while the parent drug offers a biological half-life of some 35-40 hours, prompting a once-daily dosage regimen (Lorimer, A. R., et al., J. Hum. Hypertens. 1989, 3, 191-96; Glasser, S. F. et al., A. J. H. 1989, 2, 154-57).

Amlodipine is known to exist in two chiral forms designated (S)-amlodipine and (R)-amlodipine. Importantly, the (S)-isomer is more active that the (R)-isomer. Methods of treatment using (s)-amlodipine are described in U.S. Pat. No. 6,476,058. For a review focused on amlodipine, see: Burges et al. Cardiovas Drug Dev. 1990, 8, 25-44. Amlodipine, its pharmaceutically acceptable salts, routes of administration, dosages, and formulations are described in U.S. Pat. Nos. 4,572,909 and 4,879,303. Procedures for synthesis of racemic amlodipine can be found in Arrowsmith, J. E. et al., J. Med. Chem. 1986, 29, 1696; and U.S. Pat. Nos. 4,572,909 and 5,438,145. The chemical name of (S)-amlodipine is (S)-3-ethyl-5-1-methyl-2-(2-aminoethoxymethyl)-4-(2-chlorophenyl)-1,4-dihydro-6-methyl-3,5-pyridinedicarboxylate. (S)-Amlodipine may be obtained by resolution of racemic synthetic precursors as described in WO 88/07524 and WO 88/07525. Alternatively, optically pure (S)-amlodipine can be obtained by resolution of precursors to racemic amlodipine using optically pure cinchonine and cinchonidine salts as resolving agents. See EP 0 331 315; and U.S. Published Patent Application 2002/0010200.

It has been reported in U.S. Pat. No. 6,451,826 that the optically pure (−) isomer of amlodipine is an effective antihypertensive agent for both systolic and diastolic hypertension, particularly in mild to moderate disease and angina, while avoiding various adverse effects, including but not limited to edema of the extremities, headache and dizziness, which effects are associated with the administration of the racemic mixture of amlodipine. It has also been reported that compositions of matter containing optically pure (−) amlodipine are useful in treating other conditions as may be related to the activity of (−) amlodipine as a calcium channel antagonist, including but not limited to cerebral ischemia, cerebral disorders, arrhythmias, cardiac hypertrophy, coronary vasospasm, myocardial infarction, renal impairment and acute renal failure while avoiding the above-described adverse effects associated with the administration of the racemic mixture of amlodipine. Administration of (S)-amlodipine avoids the adverse effects that are associated with the racemic mixture of amlodipine.

A variety of amlodipine salts have been reported. The besylate salt of amlodipine was first disclosed in EP 0 244 944 and has since been used worldwide in the treatment of ischaemic heart disease and hypertension. Amlodipine maleate has been described in U.S. Pat. No. 4,572,909 and J. Med. Chem. 1986, 29, 1696. It has been observed that amlodipine maleate is very sensitive to moisture and interacts with certain excipients leading to degradation. In addition, amlodipine maleate is a sticky material which poses problems during manufacturing of tablets. Procedures for the preparation of various amlodipine salts, e.g., tosylate, mesylate, succinate, acetate, and nitrate, are described in U.S. Pat. Nos. 4,879,303; 5,270,323; 5,750,707; and 6,451,826. However, these compositions relate to racemic mixtures of amlodipine or relate to salt forms of (S)-amlodipine that have limited utility as pharmaceutical agents owing to their limited solubility, poor thermal stability, or unsuitability for processing into a tablet.

In light of the superior pharmacological profile of (S)-amlodipine in comparison to racemic amlodipine, it is desirable to administer (S)-amlodipine as the therapeutic agent. Therefore, the need exists for a new salt form of (S)-amlodipine that has good physicochemical properties for the preparation of a pharmaceutical formulation. The current invention fulfills this need and has other related advantages.

### SUMMARY OF THE INVENTION

One aspect of the present invention relates to optically pure (S)-amlodipine malate. Another aspect of the present invention relates to (rac)-amlodipine malate. In a preferred embodiment, the compound is optically pure (S)-amlodipine L-malate. Another aspect of the present invention relates to a pharmaceutical composition comprising optically pure (S)-amlodipine malate. In a preferred embodiment, the pharmaceutical composition comprises optically pure (S)-amlodipine L-malate. Another aspect of the present invention relates to a method of preparing optically pure (S)-amlodipine malate, comprising admixing optically pure (S)-amlodipine with malic acid. Another aspect of the present invention relates to the various polymorphic and solvated forms of optically pure (S)-amlodipine malate. In a preferred embodiment, said malic acid is optically pure L-malic acid. In another prefered embodiment the invention relates to polymorphic and solvated forms A-G of optically pure (S)-amlodipine L-malate. The present invention also relates to a method of preparing optically pure (S)-amlodipine malate, comprising combining a salt of optically pure (S)-amlodipine with a malate salt to give optically pure (S)-amlodipine malate. In a preferred embodiment, the malate salt is an optically pure L-malate salt.

### BRIEF DESCRIPTION OF FIGURES

FIG. 1 depicts a procedure for the preparation of (S)-amlodipine L-malate (form A). If the free base of (S)-amlodipine is used as the starting material, then the first step (NaOH/MTBE) may be omitted.

FIG. 2 depicts a procedure for the preparation of (S)-amlodipine hemi-D-tartrate DMAC solvate.

FIG. 3 depicts a procedure for the preparation of (S)-amlodipine free base from (S)-amlodipine hemi-D-tartrate DMAC solvate.

FIG. 4 depicts a procedure for the preparation of (S)-amlodipine L-malate (form A) from (S)-amlodipine free base.

FIG. 5 depicts the pH solubility profile for (S)-amlodipine free base measured from (S)-amlodipine L-malate (form A) at ambient temperature.

FIG. 6 depicts solubility data for (S)-amlodipine L-malate (form A) as a function of pH at ambient temperature. The calculated solubilities of (S)-amlodipine L-malate were based on the solubilities measured for (S)-amlodipine free base derived from (S)-amlodipine maleate.

FIG. 7 depicts a plot of the natural log of (S)-amlodipine L-malate (form A) solution concentration versus time at ambient temperature.

FIG. 8 depicts a plot of rate of degradation (Log (k_{obs})) vs. pH for (S)-amlodipine L-malate (form A) at ambient temperature.

FIG. 9 depicts results of k_{obs} and percent remaining after 24 hr for (S)-amlodipine L-malate (form A).

FIG. 10 depicts a differential scanning calorimetry thermogram for (S)-amlodipine L-malate (form A).

FIG. 11 depicts hot-stage photomicrographs showing melting point range for (S)-amlodipine L-malate (form A).

FIG. 12 depicts a thermogravimetric analysis for (S)-amlodipine L-malate (form A).

FIG. 13 depicts solid state stability data for (S)-amlodipine L-malate (form A).

FIG. 14 depicts solid state stability data for (S)-amlodipine L-malate (form A).

FIG. 15 depicts a photomicrograph of (S)-amlodipine L-malate crystals (form A) with polarized light.

FIG. 16 depicts the thermal and water-association properties of various (S)-amlodipine salts. N/D means the value was not determined.

FIG. 17 depicts the aqueous solubility, reported as the free base concentration (mg/mL), of various (S)-amlodipine salts determined using an HPLC assay.

FIG. 18 depicts the X-ray Powder Diffraction Pattern for (S)-amlodipine L-malate (form A).

FIG. 19 depicts formulation composition of (S)-amlodipine L-malate (form A) tablets (5.0 mg). One mg of (S)-amlodipine is equivalent to 1.328 mg of (S)-amlodipine L-malate. [1] Acceptance Critera=90.0%-110% Label claim; [2] Acceptance Criteria=Report Results; [3] Acceptance Critera=NMT 2.0%.

FIG. 20 depicts formulation composition of (S)-amlodipine L-malate (form A) tablets (5.0 mg). One mg of (S)-amlodipine is equivalent to 1.328 mg of (S)-amlodipine L-malate. [1] Acceptance Critera=90.0%-110% Label claim; [2] Acceptance Criteria=Report Results; [3] Acceptance Critera=NMT 2.0%.

FIG. 21 depicts representative plasma concentration-time relationship after a single oral dose of a hypothetical drug. Area under the plasma concentration-time curve is indicated by shading.

FIG. 22 depicts a DSC of form A of (S)-amlodipine L-malate.

FIG. 23 depicts ¹H NMR spectra of form A of (S)-amlodipine L-malate.

FIG. 24 depicts an IR spectrum of form A of (S)-amlodipine L-malate.

FIG. 25 depicts a Raman spectrum of form A of (S)-amlodipine L-malate.

FIG. 26 depicts a TGA spectrum of form A of (S)-amlodipine L-malate.

FIG. 27 depicts a moisture sorption/desorption curve of form A of (S)-amlodipine L-malate.

FIG. 28 depicts a XRPD of amorphous (S)-amlodipine L-malate.

FIG. 29 depicts a DSC of amorphous (S)-amlodipine L-malate.

FIG. 30 depicts ¹H NMR spectra of amorphous (S)-amlodipine L-malate.

FIG. 31 depicts an IR spectra of amorphous (S)-amlodipine L-malate.

FIG. 32 depicts a Raman spectrum of amorphous (S)-amlodipine L-malate.

FIG. 33 depicts a XRPD of form B of (S)-amlodipine L-malate.

FIG. 34 depicts a DSC of form B of (S)-amlodipine L-malate.

FIG. 35 depicts a ¹H NMR spectrum of form B of (S)-amlodipine L-malate.

FIG. 36 depicts a TGA of form B of (S)-amlodipine L-malate.

FIG. 37 depicts an IR spectrum of form B of (S)-amlodipine L-malate.

FIG. 38 depicts a Raman spectrum of form B of (S)-amlodipine L-malate.

FIG. 39 depicts a XRPD of form C of (S)-amlodipine L-malate.

FIG. 40 depicts a DSC of form C of (S)-amlodipine L-malate.

FIG. 41 depicts ¹H NMR spectra of form C of (S)-amlodipine L-malate (bottom spectrum; top spectrum is form A).

FIG. 42 depicts a XRPD of form D of (S)-amlodipine L-malate.

FIG. 43 depicts a DSC of form D of (S)-amlodipine L-malate.

FIG. 44 depicts a TGA spectrum of form D of (S)-amlodipine L-malate.

FIG. 45 depicts an IR spectrum of form D of (S)-amlodipine L-malate.

FIG. 46 depicts a Raman spectrum of form D of (S)-amlodipine L-malate.

FIG. 47 depicts a XRPD of form E of (S)-amlodipine L-malate.

FIG. 48 depicts a DSC of form E of (S)-amlodipine L-malate.

FIG. 49 depicts a TGA spectra of form E of (S)-amlodipine L-malate.

FIG. 50 depicts a XRPD of form F of (S)-amlodipine L-malate.

FIG. 51 depicts a DSC of form F of (S)-amlodipine L-malate.

FIG. 52 depicts a TGA spectrum of form F of (S)-amlodipine L-malate.

FIG. 53 depicts a XRPD of form G of (S)-amlodipine L-malate.

FIG. 54 depicts a DSC of form G of (S)-amlodipine L-malate.

FIG. 55 depicts a TGA spectrum of form G of (S)-amlodipine L-malate.

FIG. 56 depicts a XRPD of form D′ of (S)-amlodipine-D-malate.

FIG. 57 depicts a DSC of form D′ of (S)-amlodipine-D-malate.

FIG. 58 depicts a TGA spectrum of form D′ of (S)-amlodipine-D-malate.

FIG. 59 depicts an IR spectrum of form D′ of (S)-amlodipine-D-malate.

FIG. 60 depicts a Raman spectrum of form D′ of (S)-amlodipine-D-malate.

FIG. 61 depicts a XRPD of (R,S)-amlodipine-D,L-malate.

FIG. 62 depicts a DSC of (R,S)-amlodipine-D,L-malate.

FIG. 63 depicts an IR spectrum of (R,S)-amlodipine-D,L-malate.

FIG. 64 depicts a ¹H NMR spectrum of (R,S)-amlodipine-D,L-malate.

FIG. 65 depicts a XRPD of (R,S)-amlodipine L-malate.

FIG. 66 depicts a DSC of (R,S)-amlodipine L-malate.

FIG. 67 depicts an TGA spectrum of (R,S)-amlodipine L-malate.

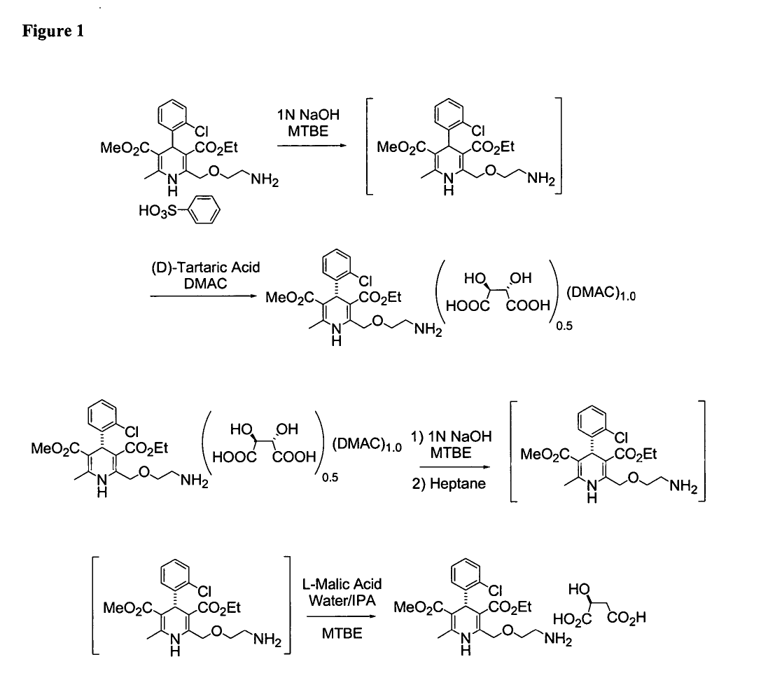

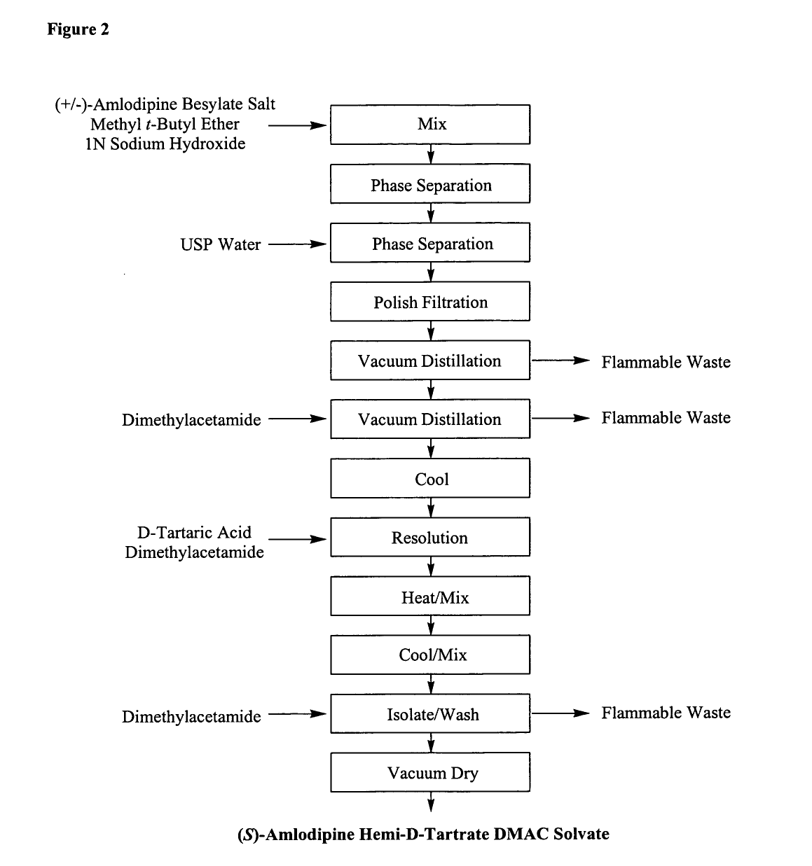

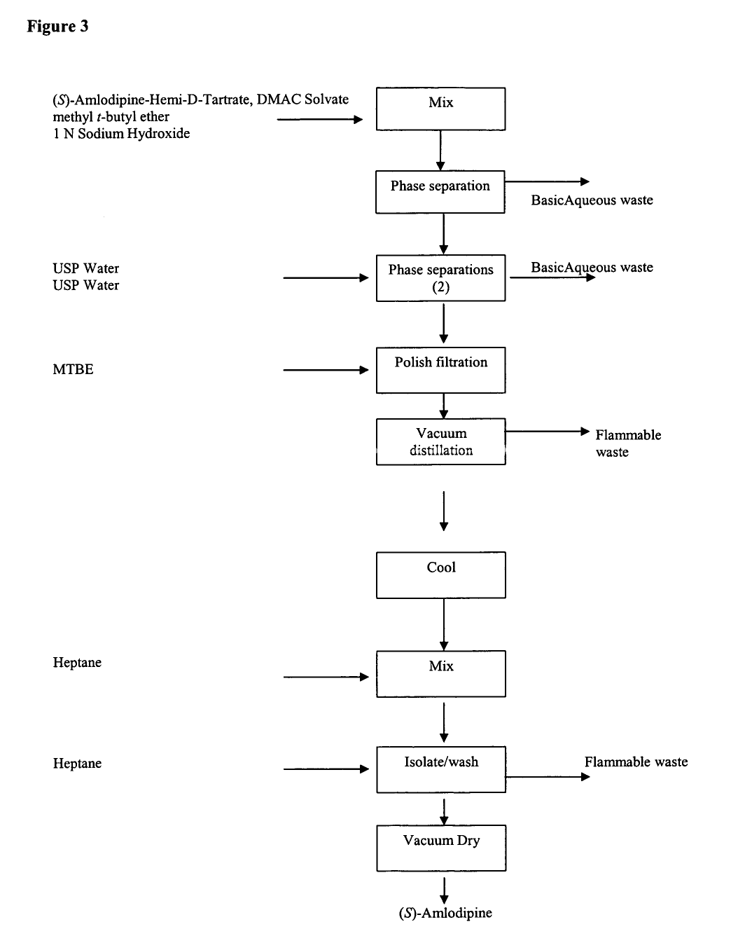

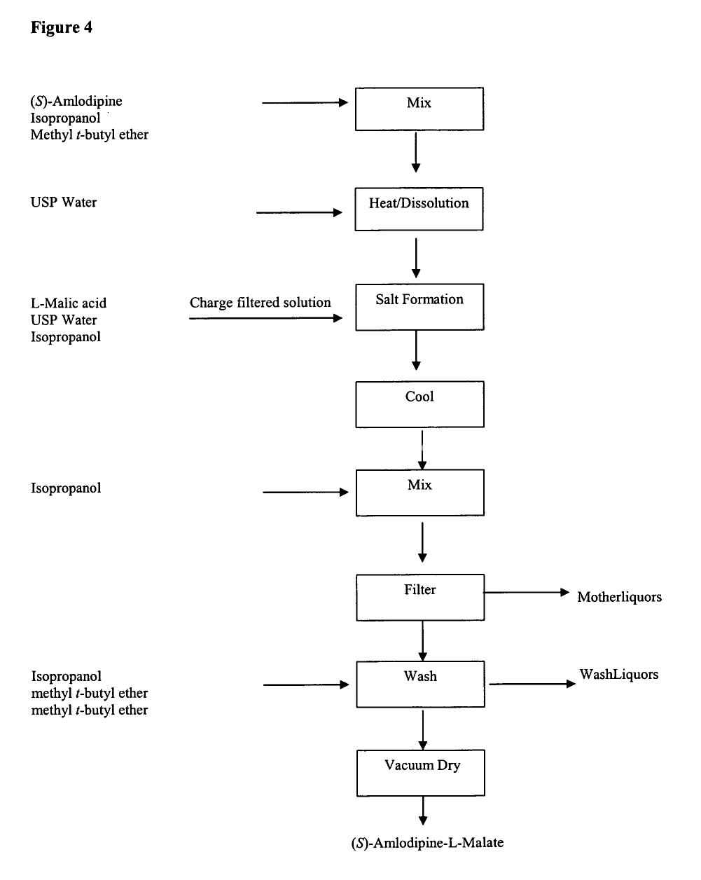

### DETAILED DESCRIPTION OF THE INVENTION

The present invention relates to (S)-amlodipine malate. In a preferred embodiment, the compound is (S)-amlodipine L-malate in one of its polymorphic or solvated forms (e.g., forms A-G). It has been discovered that (S)-amlodipine L-malate has unexpectedly superior properties as a pharmaceutical agent. (S)-Amlodipine L-malate has excellent solubility, high thermal stability, and can be easily processed into a tablet. For example, results from adhesion tests to metal surfaces, e.g., solid-dosage-form manufacturing equipment, reveal that the (S)-amlodipine L-malate, when blended at typical levels of 5% and 25% with a typical excipient, Avicel, showed decreased adhesion to solid dosage form manufacturing equipment compared to blends made with other salts. Importantly, even low-level adhesion becomes significant when manufacturing dosage forms at a typical production rate of 50,000 or more tablets or capsules per hour. Further, results from adhesion tests to metal surfaces, e.g., solid-dosage-form manufacturing equipment, reveal that adhesion of the malate salt is less than for the succinate, maleate, (D)-tartrate, and (L)-tartrate salts of (S)-amlodipine.

The adhesion test reflects the improved manufacturability of pharmaceutical dosage forms, i.e., tablets and capsules, that are made using the malate salt compared to other salts of (S)-amlodipine. Importantly, none of the previously reported salt forms of (S)-amlodipine have these critical properties. U.S. Pat. No. 6,608,206 discloses the besylate, succinate, maleate, oxalate, and tosylate salts of (S)-amlodipine. These salts were prepared by mixing (S)-amlodipine with the conjugate acid of the besylate, succinate, maleate, oxalate, and tosylate anion. For various previously reported salt forms of racemic amlodipine and (S)-amlodipine, see: U.S. Pat. Nos. 4,572,909; 4,879,303; 5,155,120; 5,270,323; 5,438,145; 5,750,707; 6,046,337; 6,046,338; 6,057,344; 6,262,092; and 6,451,826; U.S. Published Patent Applications 20030119883; 20030225143; and 20040001886; European Patent Applications EP 0 599 200; EP 0 089 167; EP 0 024 494; and EP 0 313 154; PCT Patent Applications WO 93/10779; WO 95/25722; and WO 99/52873; and Canadian Patent CA 2,188,071.

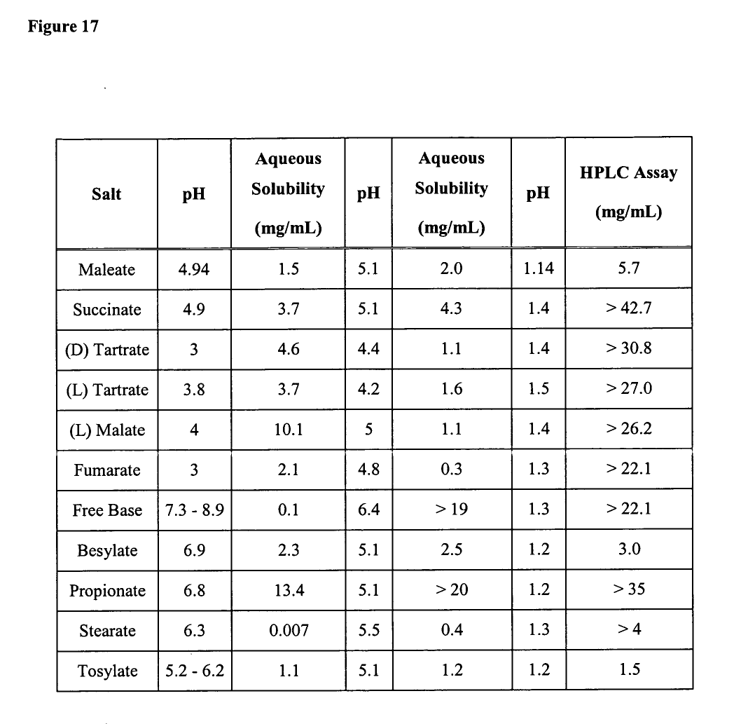

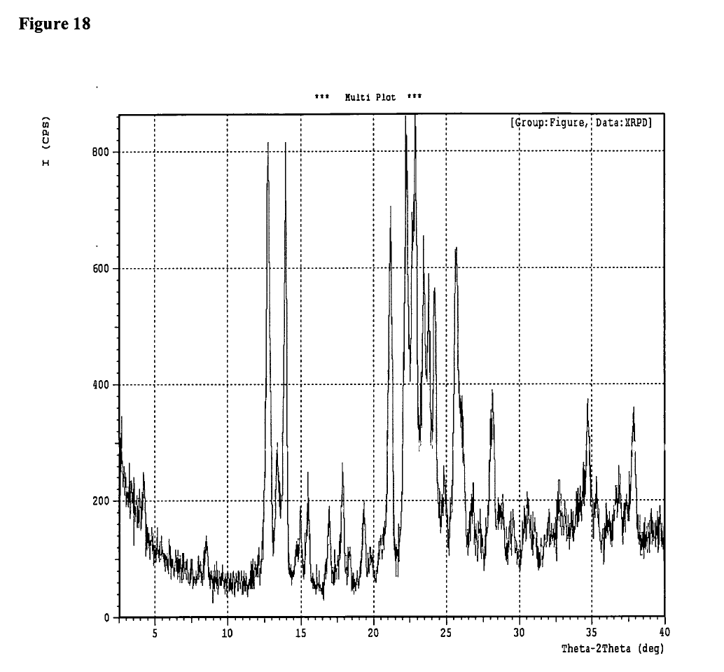

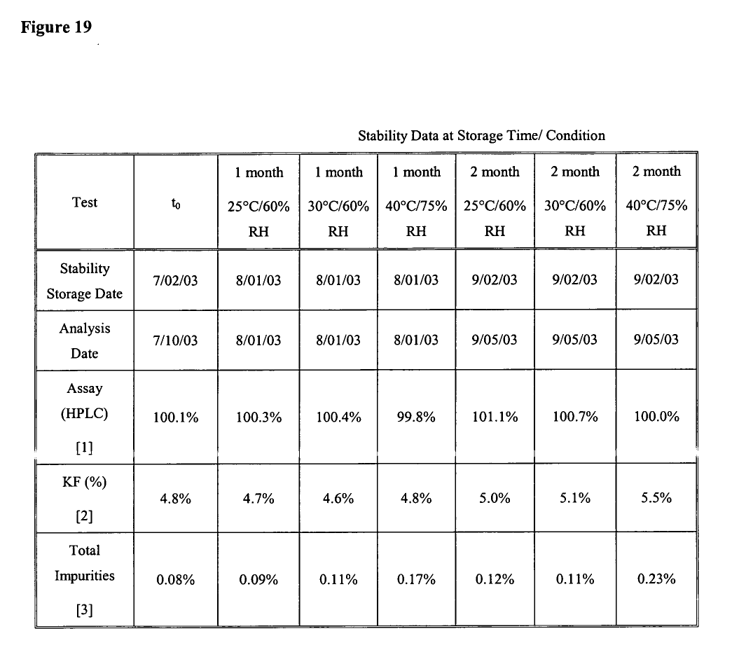

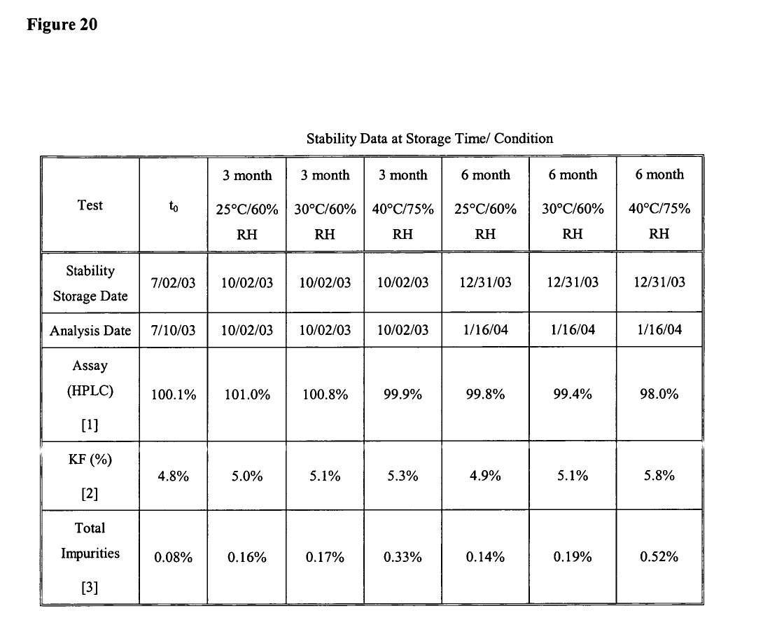

(S)-Amlodipine L-malate is unexpectedly soluble in water. For example, (S)-amlodipine L-malate has a solubility of 10.0 mg/mL at pH=3.8, and >200 mg/mL at and below pH=3.0. In contrast, (S)-amlodipine D-tartrate has a solubility of 3.7 mg/mL at pH=4.0, (S)-amlodipine maleate has a solubility of 1.5 mg/mL at pH=4.94, (S)-amlodipine maleate has a solubility of 1.5 mg/mL at pH=4.9 and 5.8 mg/mL at pH=1.1, and (S)-amlodipine besylate has a solubility of 2.5 mg/mL at pH=5.1 and 3.0 mg/mL at pH=1.2 (see FIG. 17).

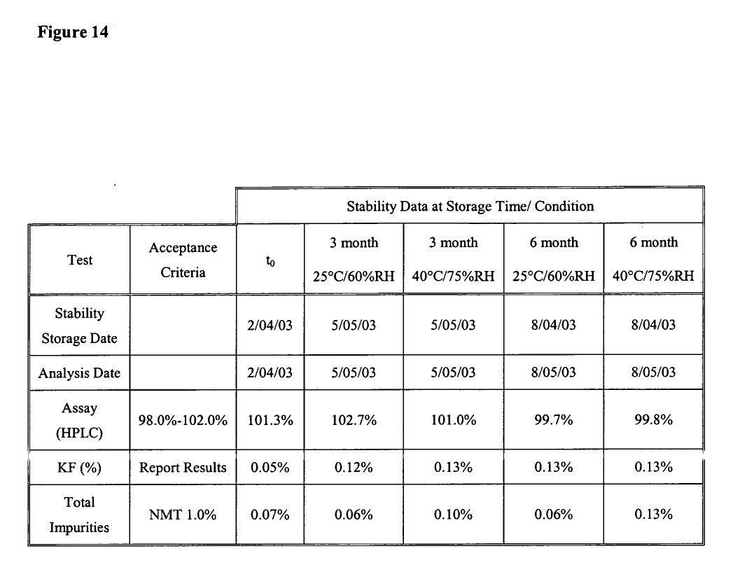

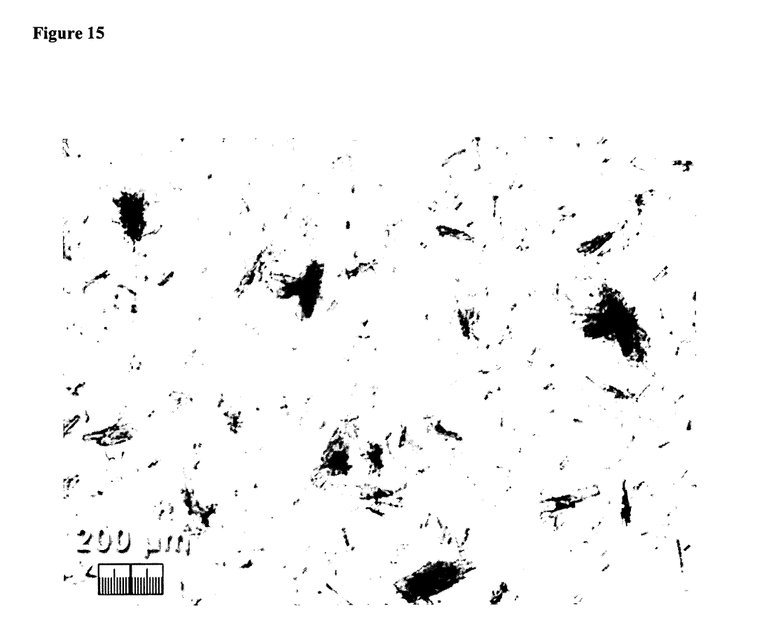

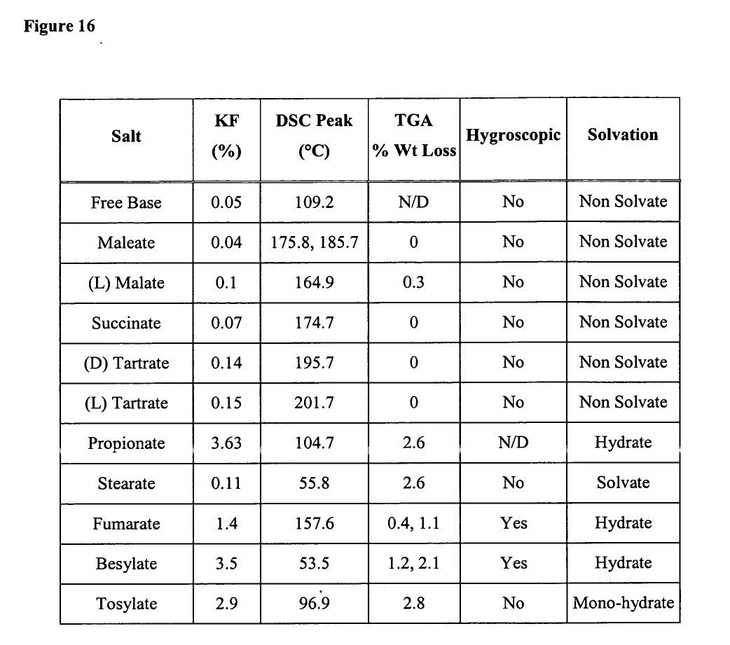

(S)-Amlodipine L-malate is unexpectedly stable at room temperature and at elevated temperatures. Solid (S)-amlodipine L-malate was stable for >6 months at 25° C./60% RH and 40° C./75% RH, as shown by KF, HPLC assay and impurity with less than 0.1% degradation observed. See FIGS. 14-15. Solid (S)-amlodipine L-malate was also fairly stable for >2 months at 60° C./75% RH, with 0.2% degradation observed.

(S)-Amlodipine L-malate has been found to be unexpectedly bioavailable in mammals (in particular humans). Analysis of plasma levels (AUC is Area Under the Curve and indicates the total amount of the drug in plasma over a period of time) of humans dosed with (S)-amlodipine-malate showed increased levels of (S)-amlodipine compared to humans dosed with equivalent amounts of (S)-amlodipine maleate. This increased bioavailability increases the effectiveness of the drug without increasing the dosage. This allows an improved effectiveness for the compound with an equivalent dose or the use of a lower dose to achieve the same efficacy.

Another aspect of the present invention relates to a pharmaceutical composition comprising (S)-amlodipine malate. In a preferred embodiment, the pharmaceutical composition comprises (S)-amlodipine L-malate. Another aspect of the present invention relates to a method of preparing (S)-amlodipine malate comprising admixing (S)-amlodipine with malic acid. In a preferred embodiment, said malic acid is L-malic acid.

Definitions

For convenience, certain terms employed in the specification, examples, and appended claims are collected here.

Certain compounds of the present invention may exist in particular geometric or stereoisomeric forms. Unless specified otherwise, the present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

As used herein, the term “optically pure” means that an active ingredient (e.g., (S)-amlodipine) for use in the compositions or methods of the present invention contain a significantly greater proportion of the specified enantiomer in relation to the non-specified enantiomer. For example, optically pure (S)-amlodipine contains a significantly greater proportion of the (S)-enantiomer in relation to the (R)-enantiomer. In a preferred embodiment, compositions including the optically pure active ingredients contain at least 90% by weight of the specified enantiomer and 10% by weight or less of the non-specified enantiomer. More preferably, such compositions contain at least 95% by weight of the specified enantiomer and 5% by weight or less of the non-specified enantiomer. Even more preferably, such compositions contain at least 99% by weight of the specified enantiomer and 1% by weight or less of the non-specified enantiomer. These percentages are based upon the total amount of the active ingredient. In instances where the modifier “optically pure” precedes the name of a substance described as a mixture of two materials (A-B), e.g., (S)-amlodipine L-malate, the term “optically pure” refers independently to each component.

The term “enantiomeric excess” is the percent excess of one enantiomer over the racemic mixture. In certain embodiments, the enantiomeric excess is greater than about 30%, 50%, or 75%. In a preferred embodiment, the enantiomeric excess is greater than about 85% or 90%. In a more preferred embodiment, the enantiomeric excess is greater than about 95% or 99%. Enantiomeric excess can be calculated using the following equation:

$$\% Enantiomeric Excess A ( ee ) = \frac{( \% Enantiomer A ) - ( \% Enantiomer B )}{( \% Enantiomer A ) + ( \% Enantiomer B )}$$

For purposes of this invention, the chemical elements are identified in accordance with the Periodic Table of the Elements, CAS version, Handbook of Chemistry and Physics, 67th Ed., 1986-87, inside cover.

The term “racemic” indicates a 1:1 ratio of enantiomers. As used herein, “(R,S)” and “(rac)” denote a 1:1 ratio of enantiomers, unless otherwise stated.

The term “heteroatom” is art-recognized and refers to an atom of any element other than carbon or hydrogen. Illustrative heteroatoms include boron, nitrogen, oxygen, phosphorus, sulfur and selenium.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure.

Unless the number of carbons is otherwise specified, “lower alkyl” refers to an alkyl group, as defined above, but having from one to about ten carbons, alternatively from one to about six carbon atoms in its backbone structure. Likewise, “lower alkenyl” and “lower alkynyl” have similar chain lengths.

The term “aralkyl” is art-recognized and refers to an alkyl group substituted with an aryl group (e.g., an aromatic or heteroaromatic group).

The terms “alkenyl” and “alkynyl” are art-recognized and refer to unsaturated aliphatic groups analogous in length and possible substitution to the alkyls described above, but that contain at least one double or triple bond respectively.

The term “aryl” is art-recognized and refers to 5-, 6- and 7-membered single-ring aromatic groups that may include from zero to four heteroatoms, for example, benzene, naphthalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are comrnon to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl”, “heteroaryl”, or “heterocyclic group” are art-recognized and refer to 3- to about 10-membered ring structures, alternatively 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxanthene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” are art-recognized and refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle” is art-recognized and refers to an aromatic or non-aromatic ring in which each atom of the ring is carbon.

The term “nitro” is art-recognized and refers to —NO₂; the term “halogen” is art-recognized and refers to —F, —Cl, —Br or —I; the term “sulfhydryl” is art-recognized and refers to —SH; the term “hydroxyl” means —OH; and the term “sulfonyl” is art-recognized and refers to —SO₂⁻. “Halide” designates the corresponding anion of the halogens, and “pseudohalide” has the definition set forth on page 560 of “Advanced Inorganic Chemistry” by Cotton and Wilkinson.

The terms “amine” and “amino” are art-recognized and refer to both unsubstituted and substituted amines, e.g., a moiety that may be represented by the general formulas: 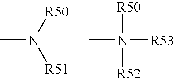
wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and refers to a moiety that may be represented by the general formula: 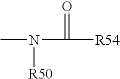
wherein R50 is as defined above, and R54 represents a hydrogen, an alkyl, an alkenyl or —(CH₂)ₘ—R61, where m and R61 are as defined above.

The term “amido” is art recognized as an amino-substituted carbonyl and includes a moiety that may be represented by the general formula: 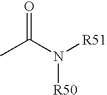
wherein R50 and R51 are as defined above. Certain embodiments of the amide in the present invention will not include imides which may be unstable.

The term “alkylthio” refers to an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carboxyl” is art recognized and includes such moieties as may be represented by the general formulas: 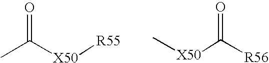
wherein X50 is a bond or represents an oxygen or a sulfur, and R55 and R56 represents a hydrogen, an alkyl, an alkenyl, —(CH₂)ₘ—R61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiolcarbonyl” group. Where X50 is a sulfur and R55 or R56 is not hydrogen, the formula represents a “thiolester.” Where X50 is a sulfur and R55 is hydrogen, the formula represents a “thiolcarboxylic acid.” Where X50 is a sulfur and R56 is hydrogen, the formula represents a “thiolformate.” On the other hand, where X50 is a bond, and R55 is not hydrogen, the above formula represents a “ketone” group. Where X50 is a bond, and R55 is hydrogen, the above formula represents an “aldehyde” group.

The term “carbamoyl” refers to —O(C═O)NRR′, where R and R′ are independently H, aliphatic groups, aryl groups or heteroaryl groups.

The term “oxo” refers to a carbonyl oxygen (═O).

The terms “oxime” and “oxime ether” are art-recognized and refer to moieties that may be represented by the general formula: 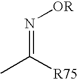
wherein R75 is hydrogen, alkyl, cycloalkyl, alkenyl, alkynyl, aryl, aralkyl, or —(CH₂)ₘ—R61. The moiety is an “oxime” when R is H; and it is an “oxime ether” when R is alkyl, cycloalkyl, alkenyl, alkynyl, aryl, aralkyl, or —(CH₂)ₘ—R61.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” is art recognized and refers to a moiety that may be represented by the general formula: 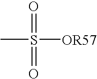
in which R57 is an electron pair, hydrogen, alkyl, cycloalkyl, or aryl.

The term “sulfate” is art recognized and includes a moiety that may be represented by the general formula: 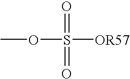
in which R57 is as defined above.

The term “sulfonamido” is art recognized and includes a moiety that may be represented by the general formula: 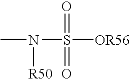
in which R50 and R56 are as defined above.

The term “sulfamoyl” is art-recognized and refers to a moiety that may be represented by the general formula: 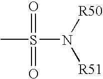
in which R50 and R51 are as defined above.

The term “sulfonyl” is art-recognized and refers to a moiety that may be represented by the general formula: 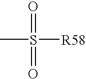
in which R58 is one of the following: hydrogen, alkyl, alkenyl, alkynyl, cycloalkyl, heterocyclyl, aryl or heteroaryl.

The term “sulfoxido” is art-recognized and refers to a moiety that may be represented by the general formula: 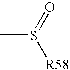
in which R58 is defined above.

The term “phosphoryl” is art-recognized and may in general be represented by the formula: 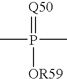
wherein Q50 represents S or O, and R59 represents hydrogen, a lower alkyl or an aryl. When used to substitute, e.g., an alkyl, the phosphoryl group of the phosphorylalkyl may be represented by the general formulas: 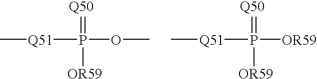
wherein Q50 and R59, each independently, are defined above, and Q51 represents O, S or N. When Q50 is S, the phosphoryl moiety is a “phosphorothioate”.

The term “phosphoramidite” is art-recognized and may be represented in the general formulas: 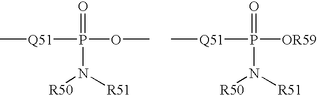
wherein Q51, R50, R51 and R59 are as defined above.

The term “phosphonamidite” is art-recognized and may be represented in the general formulas: 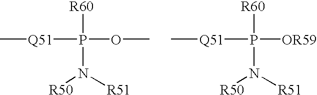
wherein Q51, R50, R51 and R59 are as defined above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The definition of each expression, e.g. alkyl, m, n, and the like, when it occurs more than once in any structure, is intended to be independent of its definition elsewhere in the same structure.

The term “selenoalkyl” is art-recognized and refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R61, m and R61 being defined above.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991). Protected forms of the inventive compounds are included within the scope of this invention.

Assessment of Bioavailablity

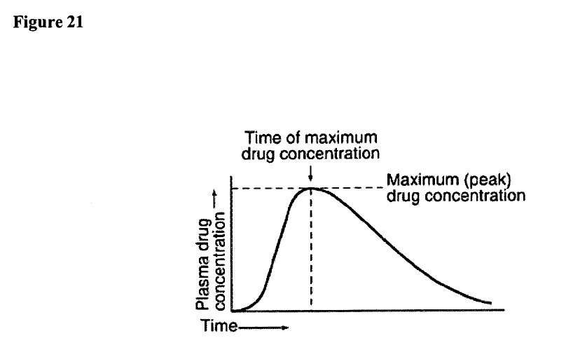

Assessment of bioavailability from plasma concentration-time data usually involves determining the maximum (peak) plasma drug concentration, the time at which maximum plasma drug concentration occurs (peak time), and the area under the plasma concentration-time curve (AUC—see FIG. 21). The plasma drug concentration increases with the extent of absorption; the peak is reached when the drug elimination rate equals absorption rate. Bioavailability determinations based on the peak plasma concentration can be misleading, because drug elimination begins as soon as the drug enters the bloodstream. The most widely used general index of absorption rate is peak time; the slower the absorption, the later the peak time. However, peak time is often not a good statistical measure because it is a discrete value that depends on frequency of blood sampling and, in the case of relatively flat concentrations near the peak, on assay reproducibility.

AUC is the most reliable measure of bioavailability. It is directly proportional to the total amount of unchanged drug that reaches the systemic circulation. For an accurate measurement, blood must be sampled frequently over a long enough time to observe virtually complete drug elimination. Drug products may be considered bioequivalent in extent and rate of absorption if their plasma-level curves are essentially superimposable. Drug products that have similar AUCs but differently shaped plasma-level curves are equivalent in extent but differ in their absorption rate-time profiles.

Compounds and Compositions of the Invention

One aspect of the present invention relates to optically pure (5)-amlodipine malate, or a polymorph, pseudopolymorph or solvate thereof.

In certain embodiments, the present invention relates to the aforementioned solvate, wherein said solvate comprises one or more solvent molecules independently selected from the group consisting of ketones, n-butanol, acetonitrile, benzonitrile, chloroform, cyclohexane, cyclopentanone, dibutylether, dichloromethane, N,N-dimethylformamide, dimethylsulfoxide, dioxane, ethanol, ethyl acetate, heptane, isopropanol, methanol, methyl ethyl ketone, N,N-dimethylacetamide, n-butylacetate, nitrobenzene, nitromethane, N-methylpyrrolidine, octane, propylene glycol, 1,2-propane diol, pyridine, tert-amylalcohol, tert-butyl methyl ether, tert-butyl methyl ether, tetrachloromethane, tetrahydrofuran, toluene, water, 2,2,2-trifluoroethanol and 2,2,4-trimethylpentane.

In certain embodiments, the present invention relates to the aforementioned solvate, wherein said solvate comprises one or more solvent molecules independently selected from the group consisting of ketones, water, dimethylsulfoxide, N-methylpyrroldinone, propylene glycol, 1,2-propane diol, pyridine, and N,N-dimethylformamide.

In certain embodiments, the present invention relates to the aforementioned solvate, wherein said solvate is a hydrate.

In certain embodiments, the present invention relates to the aforementioned compound, polymorph, pseudopolymorph or solvate, wherein the enantiomeric excess of said optically pure (S)-amlodipine is at least about 90%.

In certain embodiments, the present invention relates to the aforementioned compound, polymorph, pseudopolymorph or solvate, wherein the enantiomeric excess of said optically pure (S)-amlodipine is at least about 95%.

In certain embodiments, the present invention relates to the aforementioned compound, polymorph, pseudopolymorph or solvate, wherein the enantiomeric excess of said optically pure (S)-amlodipine is at least about 99%.

In certain embodiments, the present invention relates to the aforementioned compound, polymorph, pseudopolymorph or solvate, wherein said malate is optically pure L-malate.

In certain embodiments, the present invention relates to the aforementioned compound, polymorph, pseudopolymorph or solvate, wherein the ratio of said optically pure (S)-amlodipine to said optically pure L-malate is about 1:1.

In certain embodiments, the present invention relates to the aforementioned compound, polymorph, pseudopolymorph or solvate, wherein the ratio of said optically pure (S)-amlodipine to said optically pure L-malate is about 2:1.

In certain embodiments, the present invention relates to the aforementioned compound, polymorph, pseudopolymorph or solvate, wherein the enantiomeric excess of said optically pure L-malate is at least about 90%.

In certain embodiments, the present invention relates to the aforementioned compound, polymorph, pseudopolymorph or solvate, wherein the enantiomeric excess of said optically pure L-malate is at least about 95%.

In certain embodiments, the present invention relates to the aforementioned compound, polymorph, pseudopolymorph or solvate, wherein the enantiomeric excess of said optically pure L-malate is at least about 99%.

Another aspect of the present invention relates to optically pure (S)-amlodipine L-malate, wherein the optically pure (S)-amlodipine malate is substantially pure form A.

Another aspect of the present invention relates to a compound, solvate or hydrate comprising optically pure (S)-amlodipine L-malate, wherein said compound, solvate or hydrate has an X-ray powder diffraction spectrum substantially the same as the X-ray powder diffraction spectrum of form A.

Another aspect of the present invention relates to optically pure (S)-amlodipine L-malate, wherein the optically pure (S)-amlodipine malate is substantially pure form B.

Another aspect of the present invention relates to a compound, solvate or hydrate comprising optically pure (S)-amlodipine L-malate, wherein said compound, solvate or hydrate has an X-ray powder diffraction spectrum substantially the same as the X-ray powder diffraction spectrum of form B.

Another aspect of the present invention relates to optically pure (S)-amlodipine L-malate, wherein the optically pure (S)-amlodipine malate is substantially pure form C.

Another aspect of the present invention relates to a compound, solvate or hydrate comprising optically pure (S)-amlodipine L-malate, wherein said compound, solvate or hydrate has an X-ray powder diffraction spectrum substantially the same as the X-ray powder diffraction spectrum of form C.

Another aspect of the present invention relates to optically pure (S)-amlodipine L-malate, wherein the optically pure (S)-amlodipine malate is substantially pure form D.

Another aspect of the present invention relates to a compound, solvate or hydrate comprising optically pure (S)-amlodipine L-malate, wherein said compound, solvate or hydrate has an X-ray powder diffraction spectrum substantially the same as the X-ray powder diffraction spectrum of form D.

Another aspect of the present invention relates to optically pure (S)-amlodipine L-malate, wherein the optically pure (S)-amlodipine malate is substantially pure form E.

Another aspect of the present invention relates to a compound, solvate or hydrate comprising optically pure (S)-amlodipine L-malate, wherein said compound, solvate or hydrate has an X-ray powder diffraction spectrum substantially the same as the X-ray powder diffraction spectrum of form E.

Another aspect of the present invention relates to optically pure (S)-amlodipine L-malate, wherein the optically pure (S)-amlodipine malate is substantially pure form F.

Another aspect of the present invention relates to a compound, solvate or hydrate comprising optically pure (S)-amlodipine L-malate, wherein said compound, solvate or hydrate has an X-ray powder diffraction spectrum substantially the same as the X-ray powder diffraction spectrum of form F.

Another aspect of the present invention relates to optically pure (S)-amlodipine L-malate, wherein the optically pure (S)-amlodipine malate is substantially pure form G.

Another aspect of the present invention relates to a compound, solvate or hydrate comprising optically pure (S)-amlodipine L-malate, wherein said compound, solvate or hydrate has an X-ray powder diffraction spectrum substantially the same as the X-ray powder diffraction spectrum of form G.

In certain embodiments, the present invention relates to the aforementioned compound, polymorph, pseudopolymorph or solvate, wherein said malate is optically pure D-malate.

In certain embodiments, the present invention relates to the aforementioned compound, polymorph, pseudopolymorph or solvate, wherein the ratio of said optically pure (S)-amlodipine to said optically pure D-malate is about 1:1.

In certain embodiments, the present invention relates to the aforementioned compound, polymorph, pseudopolymorph or solvate, wherein the ratio of said optically pure (S)-amlodipine to said optically pure D-malate is about 2:1.

In certain embodiments, the present invention relates to the aforementioned compound, polymorph, pseudopolymorph or solvate, wherein the enantiomeric excess of said optically pure D-malate is at least about 90%.

In certain embodiments, the present invention relates to the aforementioned compound, polymorph, pseudopolymorph or solvate, wherein the enantiomeric excess of said optically pure D-malate is at least about 95%.

In certain embodiments, the present invention relates to the aforementioned compound, polymorph, pseudopolymorph or solvate, wherein the enantiomeric excess of said optically pure D-malate is at least about 99%.

Another aspect of the present invention relates to optically pure (S)-amlodipine D-malate, wherein the optically pure (S)-amlodipine malate is substantially pure form D′.

Another aspect of the present invention relates to a compound, solvate or hydrate comprising optically pure (S)-amlodipine D-malate, wherein said compound, solvate or hydrate has an X-ray powder diffraction spectrum substantially the same as the X-ray powder diffraction spectrum of form D′.

One aspect of the present invention relates to (rac)-amlodipine malate, or a polymorph, pseudopolymorph or solvate thereof.

In certain embodiments, the present invention relates to the aforementioned solvate, wherein said solvate comprises one or more solvent molecules independently selected from the group consisting of ketones, n-butanol, acetonitrile, benzonitrile, chloroform, cyclohexane, cyclopentanone, dibutylether, dichloromethane, N,N-dimethylformamide, dimethylsulfoxide, dioxane, ethanol, ethyl acetate, heptane, isopropanol, methanol, methyl ethyl ketone, N,N-dimethylacetamide, n-butylacetate, nitrobenzene, nitromethane, N-methylpyrrolidine, octane, propylene glycol, 1,2-propane diol, pyridine, tert-amylalcohol, tert-butyl methyl ether, tert-butyl methyl ether, tetrachloromethane, tetrahydrofuran, toluene, water, 2,2,2-trifluoroethanol and 2,2,4-trimethylpentane.

In certain embodiments, the present invention relates to the aforementioned solvate, wherein said solvate is a comprises one or more solvent molecules independently selected from the group consisting of water, dimethylsulfoxide, N-methylpyrroldinone, propylene glycol, pyridine, and N,N-dimethylformamide.

In certain embodiments, the present invention relates to the aforementioned solvate, wherein said solvate is a hydrate.

In certain embodiments, the present invention relates to the aforementioned compound, polymorph, pseudopolymorph or solvate, wherein said malate is optically pure L-malate.

In certain embodiments, the present invention relates to the aforementioned compound, polymorph, pseudopolymorph or solvate, wherein the ratio of said (rac)-amlodipine to said optically pure L-malate is about 1:1.

In certain embodiments, the present invention relates to the aforementioned compound, polymorph, pseudopolymorph or solvate, wherein the ratio of said (rac)-amlodipine to said optically pure L-malate is about 2:1.

In certain embodiments, the present invention relates to the aforementioned compound, polymorph, pseudopolymorph or solvate, wherein the enantiomeric excess of said optically pure L-malate is at least about 90%.

In certain embodiments, the present invention relates to the aforementioned compound, polymorph, pseudopolymorph or solvate, wherein the enantiomeric excess of said optically pure L-malate is at least about 95%.

In certain embodiments, the present invention relates to the aforementioned compound, polymorph, pseudopolymorph or solvate, wherein the enantiomeric excess of said optically pure L-malate is at least about 99%.

In certain embodiments, the present invention relates to the aforementioned compound, polymorph, pseudopolymorph or solvate, wherein said malate is optically pure D-malate.

In certain embodiments, the present invention relates to the aforementioned compound, polymorph, pseudopolymorph or solvate, wherein the ratio of said (rac)-amlodipine to said optically pure D-malate is about 1:1.

In certain embodiments, the present invention relates to the aforementioned compound, polymorph, pseudopolymorph or solvate, wherein the ratio of said (rac)-amlodipine to said optically pure D-malate is about 2:1.

In certain embodiments, the present invention relates to the aforementioned compound, polymorph, pseudopolymorph or solvate, wherein the enantiomeric excess of said optically pure D-malate is at least about 90%.

In certain embodiments, the present invention relates to the aforementioned compound, polymorph, pseudopolymorph or solvate, wherein the enantiomeric excess of said optically pure D-malate is at least about 95%.

In certain embodiments, the present invention relates to the aforementioned compound, polymorph, pseudopolymorph or solvate, wherein the enantiomeric excess of said optically pure D-malate is at least about 99%.

Another aspect of the present invention relates to a pharmaceutical composition comprising optically pure (S)-amlodipine malate.

In certain embodiments, the present invention relates to the aforementioned pharmaceutical composition, wherein the enantiomeric excess of said optically pure (S)-amlodipine is at least about 90%.

In certain embodiments, the present invention relates to the aforementioned pharmaceutical composition, wherein the enantiomeric excess of said optically pure (S)-amlodipine is at least about 95%.

In certain embodiments, the present invention relates to the aforementioned pharmaceutical composition, wherein the enantiomeric excess of said optically pure (S)-amlodipine is at least about 99%.

In certain embodiments, the present invention relates to the aforementioned pharmaceutical composition, wherein said malate is optically pure L-malate.

In certain embodiments, the present invention relates to the aforementioned pharmaceutical composition, wherein the ratio of said optically pure (S)-amlodipine to said optically pure L-malate is about 1:1.

In certain embodiments, the present invention relates to the aforementioned pharmaceutical composition, wherein the ratio of said optically pure (S)-amlodipine to said optically pure L-malate is about 2:1.

In certain embodiments, the present invention relates to the aforementioned pharmaceutical composition, wherein the enantiomeric excess of said optically pure L-malate is at least about 90%.

In certain embodiments, the present invention relates to the aforementioned pharmaceutical composition, wherein the enantiomeric excess of said optically pure L-malate is at least about 95%.

In certain embodiments, the present invention relates to the aforementioned pharmaceutical composition, wherein the enantiomeric excess of said optically pure L-malate is at least about 99%.

Another aspect of the present invention relates to a pharmaceutical composition comprising optically pure (S)-amlodipine malate, wherein the optically pure (S)-amlodipine malate is substantially pure form A.

Another aspect of the present invention relates to a pharmaceutical composition optically pure (S)-amlodipine malate, wherein the optically pure (S)-amlodipine malate is substantially pure form B.

Another aspect of the present invention relates to a pharmaceutical composition optically pure (S)-amlodipine malate, wherein the optically pure (S)-amlodipine malate is substantially pure form C.

Another aspect of the present invention relates to a pharmaceutical composition comprising optically pure (S)-amlodipine malate, wherein the optically pure (S)-amlodipine malate is substantially pure form D.

Another aspect of the present invention relates to a pharmaceutical composition comprising optically pure (S)-amlodipine malate, wherein the optically pure (S)-amlodipine malate is substantially pure form E.

Another aspect of the present invention relates to a pharmaceutical composition optically pure (S)-amlodipine malate, wherein the optically pure (S)-amlodipine malate is substantially pure form F.

Another aspect of the present invention relates to a pharmaceutical composition comprising optically pure (S)-amlodipine malate, wherein the optically pure (S)-amlodipine malate is substantially pure form G.

Another aspect of the present invention relates to a pharmaceutical composition comprising optically pure (S)-amlodipine malate, wherein the optically pure (S)-amlodipine malate is substantially pure form D′.

In certain embodiments, the present invention relates to the aforementioned pharmaceutical composition, wherein said malate is optically pure D-malate.

In certain embodiments, the present invention relates to the aforementioned pharmaceutical composition, wherein the ratio of said optically pure (S)-amlodipine to said optically pure D-malate is about 1:1.

In certain embodiments, the present invention relates to the aforementioned pharmaceutical composition, wherein the ratio of said optically pure (S)-amlodipine to said optically pure D-malate is about 2:1.

In certain embodiments, the present invention relates to the aforementioned pharmaceutical composition, wherein the enantiomeric excess of said optically pure D-malate is at least about 90%.

In certain embodiments, the present invention relates to the aforementioned pharmaceutical composition, wherein the enantiomeric excess of said optically pure D-malate is at least about 95%.

In certain embodiments, the present invention relates to the aforementioned pharmaceutical composition, wherein the enantiomeric excess of said optically pure D-malate is at least about 99%.

Another aspect of the present invention relates to a pharmaceutical composition comprising (rac)-amlodipine malate.

In certain embodiments, the present invention relates to the aforementioned pharmaceutical composition, wherein said malate is optically pure L-malate.

In certain embodiments, the present invention relates to the aforementioned pharmaceutical composition, wherein the ratio of said (rac)-amlodipine to said optically pure L-malate is about 1:1.

In certain embodiments, the present invention relates to the aforementioned pharmaceutical composition, wherein the ratio of said (rac)-amlodipine to said optically pure L-malate is about 2:1.

In certain embodiments, the present invention relates to the aforementioned pharmaceutical composition, wherein the enantiomeric excess of said optically pure L-malate is at least about 90%.

In certain embodiments, the present invention relates to the aforementioned pharmaceutical composition, wherein the enantiomeric excess of said optically pure L-malate is at least about 95%.

In certain embodiments, the present invention relates to the aforementioned pharmaceutical composition, wherein the enantiomeric excess of said optically pure L-malate is at least about 99%.

In certain embodiments, the present invention relates to the aforementioned pharmaceutical composition, wherein said malate is optically pure D-malate.

In certain embodiments, the present invention relates to the aforementioned pharmaceutical composition, wherein the ratio of said (rac)-amlodipine to said optically pure D-malate is about 1:1.

In certain embodiments, the present invention relates to the aforementioned pharmaceutical composition, wherein the ratio of said (rac)-amlodipine to said optically pure D-malate is about 2:1.

Methods of the Invention

One aspect of the present invention relates to a method of preparing optically pure (S)-amlodipine malate, comprising the step of admixing optically pure (S)-amlodipine with optically pure malic acid to give optically pure (S)-amlodipine malate.

In certain embodiments, the present invention relates to the aforementioned method, wherein the enantiomeric excess of said optically pure (S)-amlodipine is at least about 90%.

In certain embodiments, the present invention relates to the aforementioned method, wherein the enantiomeric excess of said optically pure (S)-amlodipine is at least about 95%.

In certain embodiments, the present invention relates to the aforementioned method, wherein the enantiomeric excess of said optically pure (S)-amlodipine is at least about 99%.

In certain embodiments, the present invention relates to the aforementioned method, wherein said malic acid is optically pure L-malic acid.

In certain embodiments, the present invention relates to the aforementioned method, wherein the ratio of said optically pure (S)-amlodipine to said optically pure L-malate of said optically pure (S)-amlodipine malate is about 1:1.

In certain embodiments, the present invention relates to the aforementioned method, wherein the ratio of said optically pure (S)-amlodipine to said optically pure L-malate of said optically pure (S)-amlodipine malate is about 2:1.

In certain embodiments, the present invention relates to the aforementioned method, wherein the enantiomeric excess of said optically pure L-malic acid is at least about 90%.

In certain embodiments, the present invention relates to the aforementioned method, wherein the enantiomeric excess of said optically pure L-malic acid is at least about 95%.

In certain embodiments, the present invention relates to the aforementioned method, wherein the enantiomeric excess of said optically pure L-malic acid is at least about 99%.

In certain embodiments, the present invention relates to the aforementioned method, wherein said malic acid is optically pure D-malic acid.

In certain embodiments, the present invention relates to the aforementioned method, wherein the ratio of said optically pure (S)-amlodipine to said optically pure D-malate of said optically pure (S)-amlodipine malate is about 1:1.

In certain embodiments, the present invention relates to the aforementioned method, wherein the ratio of said optically pure (S)-amlodipine to said optically pure D-malate of said optically pure (S)-amlodipine malate is about 2:1.

In certain embodiments, the present invention relates to the aforementioned method, wherein the enantiomeric excess of said optically pure D-malic acid is at least about 90%.

In certain embodiments, the present invention relates to the aforementioned method, wherein the enantiomeric excess of said optically pure D-malic acid is at least about 95%.

In certain embodiments, the present invention relates to the aforementioned method, wherein the enantiomeric excess of said optically pure D-malic acid is at least about 99%.

Another aspect of the present invention relates to a method of preparing optically pure (S)-amlodipine malate, comprising the steps of Example 4.

In certain embodiments, the present invention relates to the aforementioned method, wherein said malic acid is optically pure L-malic acid.

Another aspect of the present invention relates to a method of preparing optically pure (S)-amlodipine malate, comprising the step of admixing a first salt and a second salt to give optically pure (S)-amlodipine malate, wherein said first salt is a salt of optically pure (S)-amlodipine, and said second salt is an alkali metal malate, alkaline earth malate or transition metal malate.

In certain embodiments, the present invention relates to the aforementioned method, wherein said second salt is an alkali metal malate.

In certain embodiments, the present invention relates to the aforementioned method, wherein said second salt is a sodium malate or potassium malate.

In certain embodiments, the present invention relates to the aforementioned method, wherein said second salt is an alkaline earth malate.

In certain embodiments, the present invention relates to the aforementioned method, wherein said second salt is a magnesium malate or calcium malate.

In certain embodiments, the present invention relates to the aforementioned method, wherein said first salt is a (S)-amlodipine hydrohalide salt, (S)-amlodipine alkylcarboxylate salt, (S)-amlodipine hydroxyalkylcarboxylate salt, (S)-amlodipine alkenylcarboxylate salt, (S)-amlodipine arylcarboxylate salt, (S)-amlodipine aralkylcarboxylate salt, (S)-amlodipine alkyldicarboxylate salt, (S)-amlodipine hydroxyalkyldicarboxylate salt, (S)-amlodipine alkenyldicarboxylate salt, (S)-amlodipine alkyltricarboxylate salt, (S)-amlodipine hydroxyalkyltricarboxylate salt, (S)-amlodipine alkylsulfonate salt, (S)-amlodipine arylsulfonate salt, or (S)-amlodipine formate salt.

In certain embodiments, the present invention relates to the aforementioned method, wherein said first salt is (S)-amlodipine maleate, (S)-amlodipine besylate, (S)-amlodipine tosylate, (S)-amlodipine mesylate, (S)-amlodipine succinate, (S)-amlodipine salicylate, (S)-amlodipine acetate, (S)-amlodipine hemitartrate, (S)-amlodipine hydrochloride, (S)-amlodipine hydrobromide, (S)-amlodipine hydroiodide, (S)-amlodipine nitrate, (S)-amlodipine sulfate, (S)-amlodipine bisulfate, (S)-amlodipine phosphate, (S)-amlodipine lactate, (S)-amlodipine citrate, (S)-amlodipine gluconate, (S)-amlodipine ethanesulfonate, (S)-amlodipine formate, (S)-amlodipine chloroacetate, (S)-amlodipine fumarate, (S)-amlodipine benzoate, (S)-amlodipine camphorsulfonate, (S)-amlodipine mandeloate, (S)-amlodipine mucoate, (S)-amlodipine pamoate, (S)-amlodipine pantothenoate, (S)-amlodipine oxalate, or (S)-amlodipine nicotinate.

In certain embodiments, the present invention relates to the aforementioned method, wherein the enantiomeric excess said optically pure (S)-amlodipine is at least about 90%.

In certain embodiments, the present invention relates to the aforementioned method, wherein the enantiomeric excess said optically pure (S)-amlodipine is at least about 95%.

In certain embodiments, the present invention relates to the aforementioned method, wherein the enantiomeric excess said optically pure (S)-amlodipine is at least about 99%.

In certain embodiments, the present invention relates to the aforementioned method, wherein said second salt is an optically pure L-malate.

In certain embodiments, the present invention relates to the aforementioned method, wherein the ratio of said optically pure (S)-amlodipine to said optically pure L-malate of said optically pure (S)-amlodipine malate is about 1:1.

In certain embodiments, the present invention relates to the aforementioned method, wherein the ratio of said optically pure (S)-amlodipine to said optically pure L-malate of said optically pure (S)-amlodipine malate is about 2:1.

In certain embodiments, the present invention relates to the aforementioned method, wherein the enantiomeric excess said optically pure L-malate is at least about 90%.

In certain embodiments, the present invention relates to the aforementioned method, wherein the enantiomeric excess said optically pure L-malate is at least about 95%.

In certain embodiments, the present invention relates to the aforementioned method, wherein the enantiomeric excess said optically pure L-malate is at least about 99%.

In certain embodiments, the present invention relates to the aforementioned method, wherein said second salt is an optically pure D-malate.

In certain embodiments, the present invention relates to the aforementioned method, wherein the ratio of said optically pure (S)-amlodipine to said optically pure D-malate of said optically pure (S)-amlodipine malate is about 1:1.

In certain embodiments, the present invention relates to the aforementioned method, wherein the ratio of said optically pure (S)-amlodipine to said optically pure D-malate of said optically pure (S)-amlodipine malate is about 2:1.

In certain embodiments, the present invention relates to the aforementioned method, wherein the enantiomeric excess said optically pure D-malate is at least about 90%.

In certain embodiments, the present invention relates to the aforementioned method, wherein the enantiomeric excess said optically pure D-malate is at least about 95%.

In certain embodiments, the present invention relates to the aforementioned method, wherein the enantiomeric excess said optically pure D-malate is at least about 99%.

In certain embodiments, the present invention relates to the aforementioned pharmaceutical composition, wherein the enantiomeric excess of said optically pure D-malate is at least about 90%.

In certain embodiments, the present invention relates to the aforementioned pharmaceutical composition, wherein the enantiomeric excess of said optically pure D-malate is at least about 95%.

In certain embodiments, the present invention relates to the aforementioned pharmaceutical composition, wherein the enantiomeric excess of said optically pure D-malate is at least about 99%.

One aspect of the present invention relates to a method of preparing (rac)-amlodipine malate, comprising the step of admixing (rac)-amlodipine with optically pure malic acid to form (rac)-amlodipine malate.

In certain embodiments, the present invention relates to the aforementioned method, wherein said malic acid is optically pure L-malic acid.

In certain embodiments, the present invention relates to the aforementioned method, wherein the ratio of said (rac)-amlodipine to said optically pure L-malate of said (rac)-amlodipine malate is about 1:1.

In certain embodiments, the present invention relates to the aforementioned method, wherein the ratio of said (rac)-amlodipine to said optically pure L-malate of said (rac)-amlodipine malate is about 2:1.

In certain embodiments, the present invention relates to the aforementioned method, wherein the enantiomeric excess of said optically pure L-malic acid is at least about 90%.

In certain embodiments, the present invention relates to the aforementioned method, wherein the enantiomeric excess of said optically pure L-malic acid is at least about 95%.

In certain embodiments, the present invention relates to the aforementioned method, wherein the enantiomeric excess of said optically pure L-malic acid is at least about 99%.

In certain embodiments, the present invention relates to the aforementioned method, wherein said malic acid is optically pure D-malic acid.

In certain embodiments, the present invention relates to the aforementioned method, wherein the ratio of said (rac)-amlodipine to said optically pure D-malate of said (rac)-amlodipine malate is about 1:1.

In certain embodiments, the present invention relates to the aforementioned method, wherein the ratio of said (rac)-amlodipine to said optically pure D-malate of said (rac)-amlodipine malate is about 2:1.

In certain embodiments, the present invention relates to the aforementioned method, wherein the enantiomeric excess of said optically pure D-malic acid is at least about 90%.

In certain embodiments, the present invention relates to the aforementioned method, wherein the enantiomeric excess of said optically pure D-malic acid is at least about 95%.

In certain embodiments, the present invention relates to the aforementioned method, wherein the enantiomeric excess of said optically pure D-malic acid is at least about 99%.

Pharmaceutical Compositions

In another aspect, the present invention provides pharmaceutically acceptable compositions which comprise a therapeutically-effective amount of one or more of the compounds described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), capsules, tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrastemal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracistemally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, oral, intravenous, intracerebroventricular and subcutaneous doses of the compounds of this invention for a patient, when used for the indicated analgesic effects, will range from about 0.0001 to about 100 mg per kilogram of body weight per day.

If desired, the effective daily dose of the active compound may be administered as two, three, four, five, six or more sub-doses administered separately at appropriate intervals throughout the day, optionally, in unit dosage forms. Preferred dosing is one administration per day.

While it is possible for a compound of the present invention to be administered alone, it is preferable to administer the compound as a pharmaceutical formulation (composition).

The compounds according to the invention may be formulated for administration in any convenient way for use in human or veterinary medicine, by analogy with other pharmaceuticals.

In another aspect, the present invention provides pharmaceutically acceptable compositions which comprise a therapeutically-effective amount of one or more of the subject compounds, as described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin, lungs, or mucous membranes; or (4) intravaginally or intrarectally, for example, as a pessary, cream or foam; (5) sublingually or buccally; (6) ocularly; (7) transdermally; or (8) nasally.

The term “treatment” is intended to encompass also prophylaxis, therapy and cure.

The patient receiving this treatment is any animal in need, including primates, in particular humans, and other mammals such as equines, cattle, swine and sheep; and poultry and pets in general.

The compound of the invention can be administered as such or in admixtures with pharmaceutically acceptable carriers and can also be administered in conjunction with antimicrobial agents such as penicillins, cephalosporins, aminoglycosides and glycopeptides. Conjunctive therapy, thus includes sequential, simultaneous and separate administration of the active compound in a way that the therapeutical effects of the first administered one is not entirely disappeared when the subsequent is administered.

The addition of the active compound of the invention to animal feed is preferably accomplished by preparing an appropriate feed premix containing the active compound in an effective amount and incorporating the premix into the complete ration.

Alternatively, an intermediate concentrate or feed supplement containing the active ingredient can be blended into the feed. The way in which such feed premixes and complete rations can be prepared and administered are described in reference books (such as “Applied Animal Nutrition”, W.H. Freedman and CO., San Francisco, U.S.A., 1969 or “Livestock Feeds and Feeding” 0 and B books, Corvallis, Ore., U.S.A., 1977).

Micelles

Recently, the pharmaceutical industry introduced microemulsification technology to improve bioavailability of some lipophilic (water insoluble) pharmaceutical agents. Examples include Trimetrine (Dordunoo, S. K., et al., Drug Development and Industrial Pharmacy, 17(12), 1685-1713, 1991 and REV 5901 (Sheen, P. C., et al., J Pharm Sci 80(7), 712-714, 1991). Among other things, microemulsification provides enhanced bioavailability by preferentially directing absorption to the lymphatic system instead of the circulatory system, which thereby bypasses the liver, and prevents destruction of the compounds in the hepatobiliary circulation.

In one aspect of invention, the formulations contain micelles formed from a compound of the present invention and at least one amphiphilic carrier, in which the micelles have an average diameter of less than about 100 nm. More preferred embodiments provide micelles having an average diameter less than about 50 nm, and even more preferred embodiments provide micelles having an average diameter less than about 30 nm, or even less than about 20 nm.

While all suitable amphiphilic carriers are contemplated, the presently preferred carriers are generally those that have Generally-Recognized-as-Safe (GRAS) status, and that can both solubilize the compound of the present invention and microemulsify it at a later stage when the solution comes into a contact with a complex water phase (such as one found in human gastro-intestinal tract). Usually, amphiphilic ingredients that satisfy these requirements have HLB (hydrophilic to lipophilic balance) values of 2-20, and their structures contain straight chain aliphatic radicals in the range of C-6 to C-20. Examples are polyethylene-glycolized fatty glycerides and polyethylene glycols.

Particularly preferred amphiphilic carriers are saturated and monounsaturated polyethyleneglycolyzed fatty acid glycerides, such as those obtained from fully or partially hydrogenated various vegetable oils. Such oils may advantageously consist of tri-. di- and mono-fatty acid glycerides and di- and mono-polyethyleneglycol esters of the corresponding fatty acids, with a particularly preferred fatty acid composition including capric acid 4-10, capric acid 3-9, lauric acid 40-50, myristic acid 14-24, palmitic acid 4-14 and stearic acid 5-15%. Another useful class of amphiphilic carriers includes partially esterified sorbitan and/or sorbitol, with saturated or mono-unsaturated fatty acids (SPAN-series) or corresponding ethoxylated analogs (TWEEN-series).

Commercially available amphiphilic carriers are particularly contemplated, including Gelucire-series, Labrafil, Labrasol, or Lauroglycol (all manufactured and distributed by Gattefosse Corporation, Saint Priest, France), PEG-mono-oleate, PEG-di-oleate, PEG-mono-laurate and di-laurate, Lecithin, Polysorbate 80, etc (produced and distributed by a number of companies in USA and worldwide).

Polymers

Hydrophilic polymers suitable for use in the present invention are those which are readily water-soluble, can be covalently attached to a vesicle-forming lipid, and which are tolerated in vivo without toxic effects (i.e., are biocompatible). Suitable polymers include polyethylene glycol (PEG), polylactic (also termed polylactide), polyglycolic acid (also termed polyglycolide), a polylactic-polyglycolic acid copolymer, and polyvinyl alcohol. Preferred polymers are those having a molecular weight of from about 100 or 120 daltons up to about 5,000 or 10,000 daltons, and more preferably from about 300 daltons to about 5,000 daltons. In a particularly preferred embodiment, the polymer is polyethyleneglycol having a molecular weight of from about 100 to about 5,000 daltons, and more preferably having a molecular weight of from about 300 to about 5,000 daltons. In a particularly preferred embodiment, the polymer is polyethyleneglycol of 750 daltons (PEG(750)). Polymers may also be defined by the number of monomers therein; a preferred embodiment of the present invention utilizes polymers of at least about three monomers, such PEG polymers consisting of three monomers (approximately 150 daltons).

Other hydrophilic polymers which may be suitable for use in the present invention include polyvinylpyrrolidone, polymethoxazoline, polyethyloxazoline, polyhydroxypropyl methacrylamide, polymethacrylamide, polydimethylacrylamide, and derivatized celluloses such as hydroxymethylcellulose or hydroxyethylcellulose.

In certain embodiments, a formulation of the present invention comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

Cyclodextrins

Cyclodextrins are cyclic oligosaccharides, consisting of 6, 7 or 8 glucose units, designated by the Greek letter .alpha., beta. or .gamma., respectively. Cyclodextrins with fewer than six glucose units are not known to exist. The glucose units are linked by alpha-1,4-glucosidic bonds. As a consequence of the chair conformation of the sugar units, all secondary hydroxyl groups (at C-2, C-3) are located on one side of the ring, while all the primary hydroxyl groups at C-6 are situated on the other side. As a result, the external faces are hydrophilic, making the cyclodextrins water-soluble. In contrast, the cavities of the cyclodextrins are hydrophobic, since they are lined by the hydrogen of atoms C-3 and C-5, and by ether-like oxygens. These matrices allow complexation with a variety of relatively hydrophobic compounds, including, for instance, steroid compounds such as 17.beta.-estradiol (see, e.g., van Uden et al. Plant Cell Tiss. Org. Cult. 38:1-3-113 (1994)). The complexation takes place by Van der Waals interactions and by hydrogen bond formation. For a general review of the chemistry of cyclodextrins, see, Wenz, Agnew. Chem. Int. Ed. Engl., 33:803-822 (1994).

The physico-chemical properties of the cyclodextrin derivatives depend strongly on the kind and the degree of substitution. For example, their solubility in water ranges from insoluble (e.g., triacetyl-beta-cyclodextrin) to 147% soluble (w/v) (G-2-beta-cyclodextrin). In addition, they are soluble in many organic solvents. The properties of the cyclodextrins enable the control over solubility of various formulation components by increasing or decreasing their solubility.

Numerous cyclodextrins and methods for their preparation have been described. For example, Parmeter (I), et al. (U.S. Pat. No. 3,453,259) and Gramera, et al. (U.S. Pat. No. 3,459,731) described electroneutral cyclodextrins. Other derivatives include cyclodextrins with cationic properties [Parmeter (II), U.S. Pat. No. 3,453,257], insoluble crosslinked cyclodextrins (Solms, U.S. Pat. No. 3,420,788), and cyclodextrins with anionic properties [Parmeter (III), U.S. Pat. No. 3,426,011]. Among the cyclodextrin derivatives with anionic properties, carboxylic acids, phosphorous acids, phosphinous acids, phosphonic acids, phosphoric acids, thiophosphonic acids, thiosulphinic acids, and sulfonic acids have been appended to the parent cyclodextrin [see, Parmeter (III), supra]. Furthermore, sulfoalkyl ether cyclodextrin derivatives have been described by Stella, et al. (U.S. Pat. No. 5,134,127).

Liposomes

Liposomes consist of at least one lipid bilayer membrane enclosing an aqueous internal compartment. Liposomes may be characterized by membrane type and by size. Small unilamellar vesicles (SUVs) have a single membrane and typically range between 0.02 and 0.05 μm in diameter; large unilamellar vesicles (LUVS) are typically larger than 0.05 μm Oligolamellar large vesicles and multilamellar vesicles have multiple, usually concentric, membrane layers and are typically larger than 0.1 μm. Liposomes with several nonconcentric membranes, i.e., several smaller vesicles contained within a larger vesicle, are termed multivesicular vesicles.

One aspect of the present invention relates to formulations comprising liposomes containing a compound of the present invention, where the liposome membrane is formulated to provide a liposome with increased carrying capacity. Alternatively or in addition, the compound of the present invention may be contained within, or adsorbed onto, the liposome bilayer of the liposome. The compound of the present invention may be aggregated with a lipid surfactant and carried within the liposome's internal space; in these cases, the liposome membrane is formulated to resist the disruptive effects of the active agent-surfactant aggregate.

According to one embodiment of the present invention, the lipid bilayer of a liposome contains lipids derivatized with polyethylene glycol (PEG), such that the PEG chains extend from the inner surface of the lipid bilayer into the interior space encapsulated by the liposome, and extend from the exterior of the lipid bilayer into the surrounding environment.

Active agents contained within liposomes of the present invention are in solubilized form. Aggregates of surfactant and active agent (such as emulsions or micelles containing the active agent of interest) may be entrapped within the interior space of liposomes according to the present invention. A surfactant acts to disperse and solubilize the active agent, and may be selected from any suitable aliphatic, cycloaliphatic or aromatic surfactant, including but not limited to biocompatible lysophosphatidylcholines (LPCs) of varying chain lengths (for example, from about C₁₄ to about C₂₀). Polymer-derivatized lipids such as PEG-lipids may also be utilized for micelle formation as they will act to inhibit micelle/membrane fusion, and as the addition of a polymer to surfactant molecules decreases the CMC of the surfactant and aids in micelle formation. Preferred are surfactants with CMCs in the micromolar range; higher CMC surfactants may be utilized to prepare micelles entrapped within liposomes of the present invention, however, micelle surfactant monomers could affect liposome bilayer stability and would be a factor in designing a liposome of a desired stability.

Liposomes according to the present invention may be prepared by any of a variety of techniques that are known in the art. See, e.g., U.S. Pat. No. 4,235,871; Published PCT applications WO 96/14057; New RRC, Liposomes: A practical approach, IRL Press, Oxford (1990), pages 33-104; Lasic DD, Liposomes from physics to applications, Elsevier Science Publishers BV, Amsterdam, 1993.

For example, liposomes of the present invention may be prepared by diffusing a lipid derivatized with a hydrophilic polymer into preformed liposomes, such as by exposing preformed liposomes to micelles composed of lipid-grafted polymers, at lipid concentrations corresponding to the final mole percent of derivatized lipid which is desired in the liposome. Liposomes containing a hydrophilic polymer can also be formed by homogenization, lipid-field hydration, or extrusion techniques, as are known in the art.

In another exemplary formulation procedure, the active agent is first dispersed by sonication in a lysophosphatidylcholine or other low CMC surfactant (including polymer grafted lipids) that readily solubilizes hydrophobic molecules. The resulting micellar suspension of active agent is then used to rehydrate a dried lipid sample that contains a suitable mole percent of polymer-grafted lipid, or cholesterol. The lipid and active agent suspension is then formed into liposomes using extrusion techniques as are known in the art, and the resulting liposomes separated from the unencapsulated solution by standard column separation.

In one aspect of the present invention, the liposomes are prepared to have substantially homogeneous sizes in a selected size range. One effective sizing method involves extruding an aqueous suspension of the liposomes through a series of polycarbonate membranes having a selected uniform pore size; the pore size of the membrane will correspond roughly with the largest sizes of liposomes produced by extrusion through that membrane. See e.g., U.S. Pat. No. 4,737,323 (Apr. 12, 1988).

Release Modifiers

The release characteristics of a formulation of the present invention depend on the encapsulating material, the concentration of encapsulated drug, and the presence of release modifiers. For example, release can be manipulated to be pH dependent, for example, using a pH sensitive coating that releases only at a low pH, as in the stomach, or a higher pH, as in the intestine. An enteric coating can be used to prevent release from occurring until after passage through the stomach. Multiple coatings or mixtures of cyanamide encapsulated in different materials can be used to obtain an initial release in the stomach, followed by later release in the intestine. Release can also be manipulated by inclusion of salts or pore forming agents, which can increase water uptake or release of drug by diffusion from the capsule. Excipients which modify the solubility of the drug can also be used to control the release rate. Agents which enhance degradation of the matrix or release from the matrix can also be incorporated. They can be added to the drug, added as a separate phase (i.e., as particulates), or can be co-dissolved in the polymer phase depending on the compound. In all cases the amount should be between 0.1 and thirty percent (w/w polymer). Types of degradation enhancers include inorganic salts such as ammonium sulfate and ammonium chloride, organic acids such as citric acid, benzoic acid, and ascorbic acid, inorganic bases such as sodium carbonate, potassium carbonate, calcium carbonate, zinc carbonate, and zinc hydroxide, and organic bases such as protamine sulfate, spermine, choline, ethanolamine, diethanolamine, and triethanolamine and surfactants such as Tween® and Pluronic®. Pore forming agents which add microstructure to the matrices (i.e., water soluble compounds such as inorganic salts and sugars) are added as particulates. The range should be between one and thirty percent (w/w polymer).

Uptake can also be manipulated by altering residence time of the particles in the gut. This can be achieved, for example, by coating the particle with, or selecting as the encapsulating material, a mucosal adhesive polymer. Examples include most polymers with free carboxyl groups, such as chitosan, celluloses, and especially polyacrylates (as used herein, polyacrylates refers to polymers including acrylate groups and modified acrylate groups such as cyanoacrylates and methacrylates).

### EXEMPLIFICATION

The invention now being generally described, it will be more readily understood by reference to the following examples, which are included merely for purposes of illustration of certain aspects and embodiments of the present invention, and are not intended to limit the invention.

### Example 1

### Process Description for (S)-Amlodipine Hemi-D-Tartrate DMAC solvate from (RS)-Amlodipine Besylate

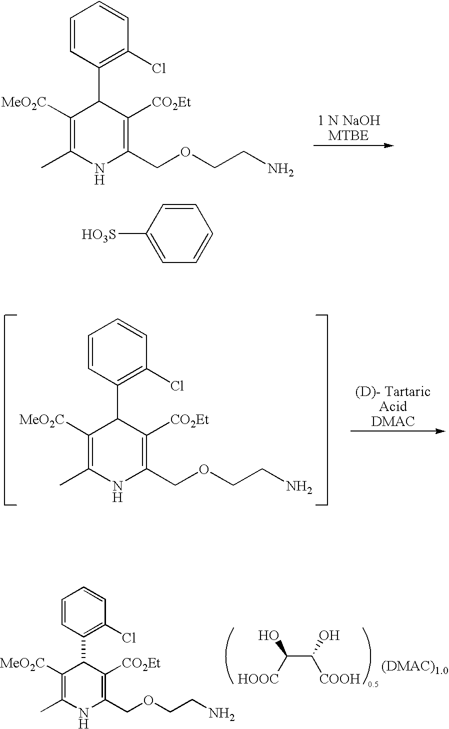

(RS)-Amlodipine besylate (49.8 kg) and methyl t-butyl ether (MTBE) (240 kg) were charged to a 200 gal reactor, followed by the addition of aqueous 1 N sodium hydroxide (137 kg). The mixture was agitated for 20 to 30 minutes and then the layers were allowed to separate for a minimum of 15 minutes. The aqueous layer was removed and the organic layer was washed twice with water (about 66 kg each). The organic layer was polish filtered and concentrated under vacuum (at not more than about 50° C.) to about 109 L. N,N-Dimethylacetamide (DMAC, 153 kg) was charged to the reactor and the solution was again concentrated under vacuum until the batch temperature reached 45 to 55° C. The final volume was about 208 L. The reaction was cooled to 20 to 25° C., followed by the addition of a D-tartaric acid solution (14 kg of D-tartaric acid in about 153 kg of DMAC) over 20 to 30 minutes. The mixture was heated to 68 to 72° C. over about 1 hour and held at this temperature for about 1 hour. The reaction mixture was cooled to 21 to 23° C. over 2 to 3 hours and agitated at this temperature for 30 to 40 minutes. The slurry was filtered, and the cake was washed with DMAC (about 76 kg) and MTBE (about 60 kg). The wet cake (20.3 kg) was dried in a vacuum dryer for a minimum of 6 hours at 45 to 50° C. to yield 20.1 kg of (S)-Amlodipine Hemi-D-Tartrate DMAC solvate (chemical purity 99.9%, 98.6% ee).

Note: The free base can be used as a starting point in the scheme as well by eliminating the sodium hydroxide/MTBE step and continuing as written. See Example 2.

### Example 2

### Process Description for (S)-Amlodpine-hemi-D-Tartrate DMAC Solvate from (RS)-Amlodipine Free Base

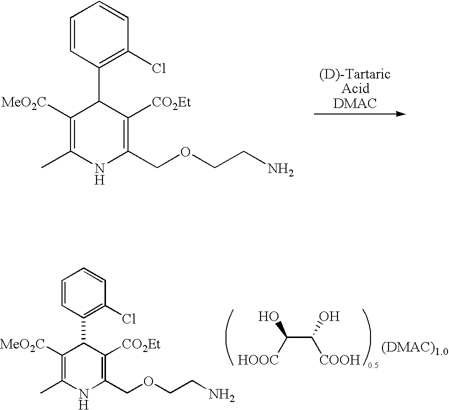

A solution of D-tartaric acid (9.5 kg, 63.2 moles) in DMAC (104 kg) was added to a slurry of (RS)-amlodipine free-base (25 kg, 61 moles) in DMAC (104 kg). The reaction mixture was agitated and heated to about 70° C. The reaction mass was held for about one hour with agitation at about 70° C. The resulting slurry was then cooled with agitation to about 22° C. over 2.5 to 3 hours (cooling profile was about 0.3° C./min). The slurry was held with agitation at about 22° C. for about 0.5 hr. The solid was isolated by filtration, washed by re-slurrying with DMAC followed by a displacement wash with MTBE. The wet cake was vacuum dried at about 45° C. to produce (S)-Amlodipine-hemi-D-Tartrate-DMAC solvate (13.9 kg, 99.8% chemical purity, 99.2% ee).

### Example 3

### Process Description for (S)-Amlodipine Free Base from (S)-Amlodipine Hemi-D-Tartrate DMAC solvate

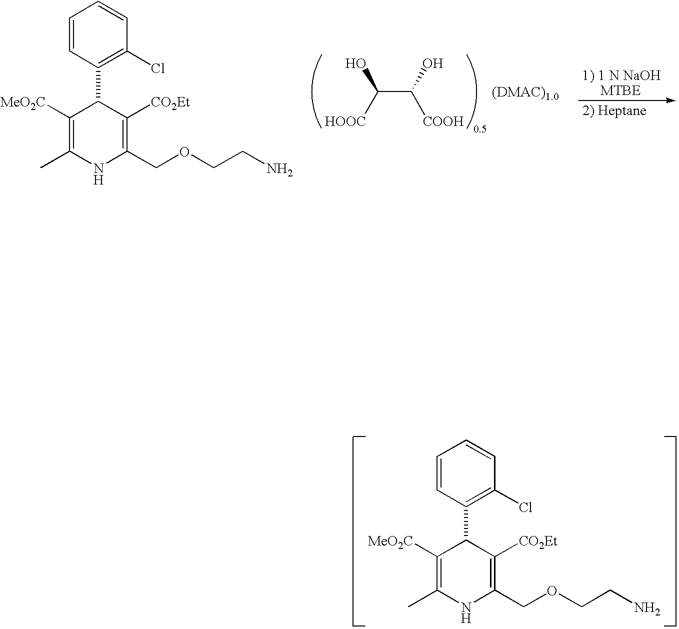

(S)-Amlodipine hemi-D-tartrate DMAC solvate (30 kg) and MTBE (about 245 kg) were charged to a 200 gal reactor. The temperature was adjusted to 20 to 25° C., followed by the addition of 1 N sodium hydroxide (about 86 kg) while maintaining a temperature of 20 to 25° C. The reaction was stirred for about 30 minutes and then the layers were allowed to separate. The bottom aqueous layer was removed, and the organic layer was washed twice with water (about 82 kg each wash). The solution was filtered through a polishing filter, followed by a reactor and line rinse of MTBE (about 45 kg). The solution was distilled to about 87 L under vacuum (jacket temperature not more than about 40° C.) and the mixture was cooled to 20 to 25° C. Heptane (about 80 kg) was charged over about 60 minutes and the reaction was agitated at 20 to 25° C. for about 60 minutes. The slurry was filtered and washed with heptane (about 131 kg). The wet cake (21.2 kg) was vacuum dried at 40 to 50° C. to yield 19.7 kg of (S)-Amlodipine free-base (99.97% chemical purity, 99.9% ee).

### Example 4

### Process Description for (S)-Amlodipine L-Malate from (S)-Amlodipine Free Base

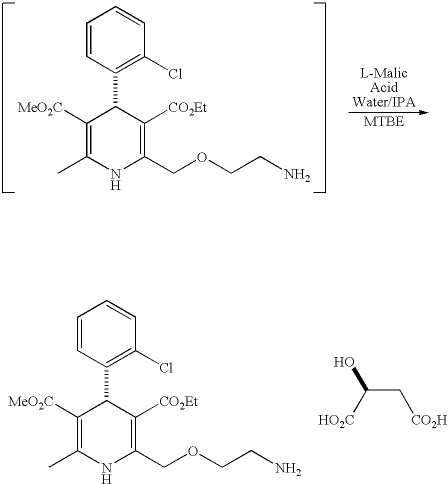

L-Malic acid (6.7 kg), water (5.7 kg) and isopropyl alcohol (17 kg) were charged to a suitable mixing vessel and mixed until a solution was obtained. The L-malic acid solution was filtered through a polishing filter into a suitable container and held for later use. (S)-Amlodipine free base (19.5 kg), isopropyl alcohol (about 142 kg) and MTBE (about 15 kg) were charged to a 200 gal reactor. The temperature was adjusted to 48 to 52° C. over about 30 minutes. The previously prepared L-malic acid solution was added to the solution of (S)-amlodipine free-base over 15 to 20 minutes while maintaining the temperature at 48 to 52° C. The reaction was stirred at 48 to 52° C. for about 1 hour, and cooled to about 0° C. over 2 to 3 hours and held for a minimum of about 1 hour at that temperature. The slurry was filtered and washed with IPA (about 44 kg) and twice with MTBE (about 44 kg each wash). The wet cake (28.9 kg) was vacuum dried at about 60° C. to a constant weight. The isolated dried yield was 25.4 kg of (S)-amlodipine L-malate (99.97% chemical purity, 99.7% ee, 0.06% water). The resulting (S)-amlodipine L-malate is polymorphic form A.

### Example 5

### Characterization of (S)-Amlodipine Malate (polymorphic form A)

1. Partition Coefficient and pKa Determinations

The GLpKa potentiometric system uses expert system software to calculate and refine the pKa and Log P from simple acid and base titrations of chemical substances. The pKa was determined by dissolving (S)-amlodipine L-malate in 0.15 M KCl followed by base titrant addition to increase the pH to the desired starting point. The solution was then titrated with acid to the final pH. The pH range of the experiment was 3-11. An approximate pKa was determined, and refined to a final value using the expert system software. The Log P was determined by titration in the presence of octanol. The pKa in water and the pKa in the presence of octanol were compared, and the Log P value was calculated (Avdeef A. Quant. Struct.-Act. Relate. 1992, 11, 510).

The partition coefficient (P) and pKa (ionization constant) were determined using the Sirius GLpKa potentiometric-titration system: pKa=9.19±0.20; Log P=−2.67±0.40 (pH 5.0); Log P=−0.28±0.40 (pH 7.4). Samples were analyzed in duplicate. Log P in the current context is used to describe partitioning of both charged and uncharged forms of the test article; calculated value shown (lower limit of detection for log P is 0).

2. Wettability

(S)-Amlodipine L-malate exhibits good wettability in DI water and 100 mM aqueous buffers. Solid (S)-amlodipine L-malate was observed to penetrate through the surface of the liquid very easily and disperse well during saturation of aqueous solvents.

3. Water Solubility

A saturated solution with excess solid (S)-amlodipine L-malate was prepared in a 20 mL clear glass scintillation vial with a Teflon lined screw top, and allowed to mix at ambient temperature for 5 days (3 confirmatory values). The solution was sampled periodically, filtered using a 0.2 μm PTFE syringe filter and assayed by HPLC.

A saturated solution with excess solid (S)-amlodipine L-malate was prepared and allowed to mix overnight at ambient temperature. The sample was filtered through a 0.2 μm filter and assayed by HPLC.

TABLE 1
Solubility and pH of (S)-Amlodipine-L-Malate in DI H₂O

| Free Base | | |
|---|---|---|
| Concentration | Malate Concentration | |
| (mg/mL) | (mg/mL) | pH |
| 6.0 | 8.0 | 3.56 |
| 10.1 | 13.4 | 4.0 |

(S)-Amlodipine L-malate is slightly soluble in de-ionized H₂O at ambient temperature. The water solubility value from an initial salt screen is higher than the value generated with a more recent sample. The variability noted may be due to differences in equilibration times between both experiments and/or quality of the sample from the initial salt screen.

4. pH Solubility Profile

Saturated solutions with excess solid (S)-amlodipine L-malate were prepared in 20 mL clear glass scintillation vials with Teflon lined screw tops, and allowed to mix at ambient temperature for the course of the experiment (3 confirmatory values when possible). Samples at pH 3.8, 4.5, and 5.0 were monitored over 1 week. Samples at pH 6.0, 6.8, and 7.5 were monitored over 4 days. The sample at pH 8.0 was monitored over 2 days. The samples with apparent solubility >100 mg/mL were not assayed. The samples at pH 3.8 or greater were filtered using 0.2 μm PTFE syringe filters and assayed by HPLC.

Saturated solutions with excess solid (S)-amlodipine L-malate were prepared and allowed to mix overnight at ambient temperature. The samples were filtered through a 0.2 μm filter and assayed by HPLC.

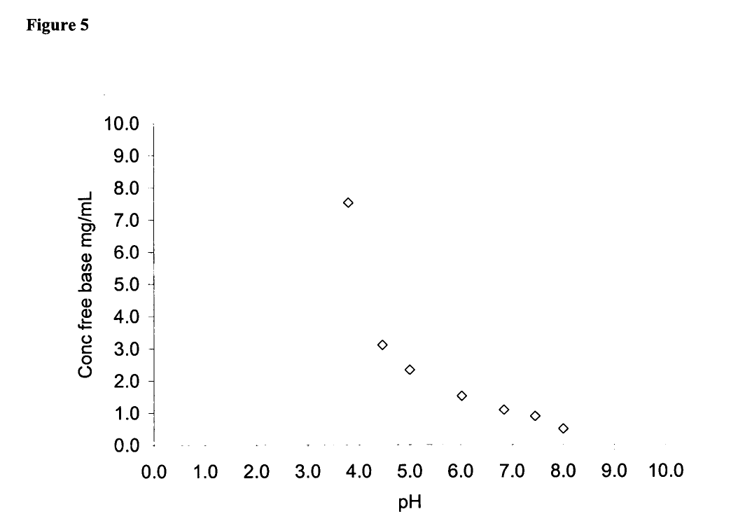

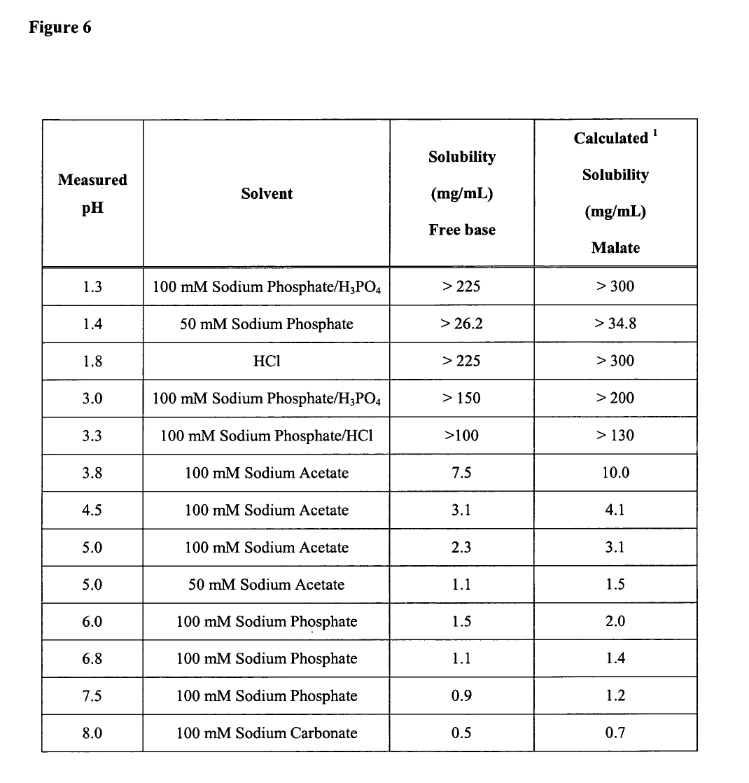

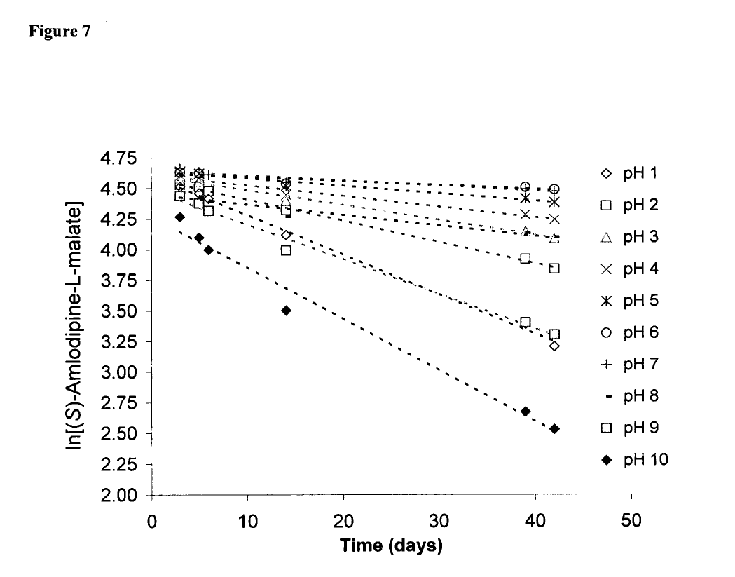

The data (FIG. 5) follow the theoretical profile between pH 3.8 and pH 8.0. Solubility values increase to >100 mg/mL below pH 3.8 and were not determined quantitatively. See FIGS. 5 and 6.

5. Solubility in Methanol, Ethanol, and Isopropanol

Excess solid (S)-amlodipine L-malate was added to methanol, ethanol, and isopropanol and the suspensions were vortexed to disperse the solid. The suspensions were placed on an orbital shaker, and allowed to shake at room temperature for 1 week. Samples were periodically filtered, diluted and assayed by HPLC.

TABLE 2
Alcohol Solubility Data for (S)-Amlodipine-L-Malate

| | Free Base Concentration | Malate Concentration |
|---|---|---|
| Solvent | (mg/mL) | (mg/mL) |
| Methanol | 49.4 | 65.7 |
| Ethanol | 5.3 | 7.1 |
| Isopropanol | 0.7 | 0.9 |

6. pH Stability Profile

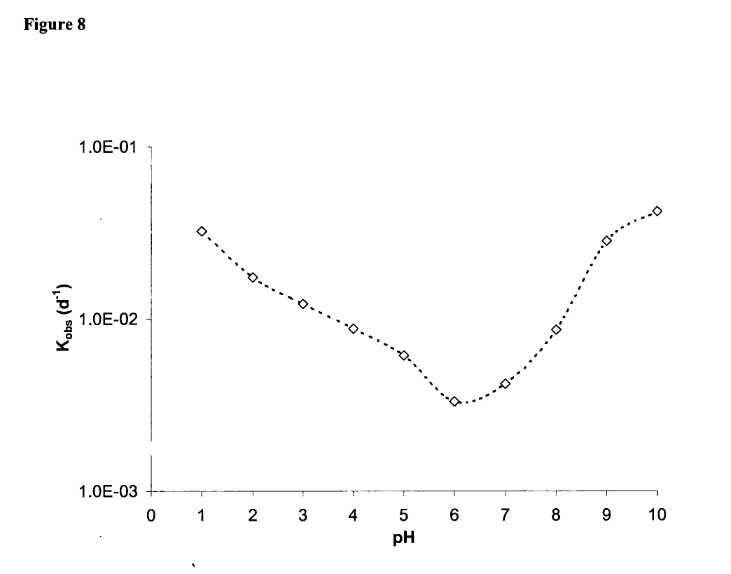

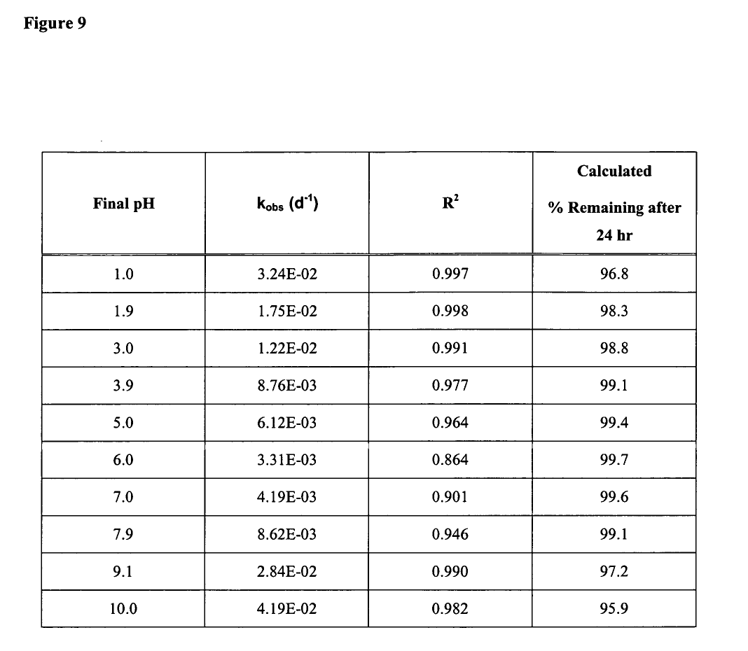

Solutions of (S)-amlodipine L-malate at ˜0.05 mg/mL were made in aqueous solvents: HCl (pH 1.0); 50 mM sodium phosphate (pH 2.0, 3.0, 6.0, 7.0); 50 mM sodium acetate (pH 3.9, 5.0); and sodium carbonate (pH 7.9, 9.1, 10.0). These solutions were stored in the carousel of the Waters LCM HPLC at ambient temperature. Solutions were analyzed by HPLC over a 42-day period (FIGS. 8 and 9). These results establish that (S)-Amlodipine-L-Malate is most stable in solution at pH 6 when stored at ambient temperature.

7. DSC and Hot Stage Microscopy

Differential Scanning Calorimetry (DSC): Samples were analyzed using a Perkin Elmer DSC 7 differential scanning calorimeter with a heating rate of 10° C./min. Each sample was analyzed in a sealed pan with a pinhole. Hotstage Microscopy: Samples were analyzed using the Nikon Microphot Polarized Light Microscope equipped with a Linkam Hotstage THMS 600. Each sample was placed on a cover slip, located on hotstage furnace, insulated from above by 2 layers (2 cover slips with air space between each layer) and hotstage cover, and heated at a rate of 10° C./min.

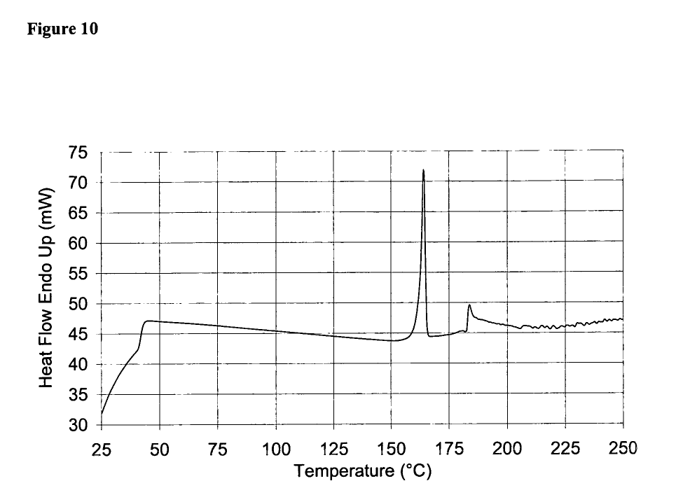

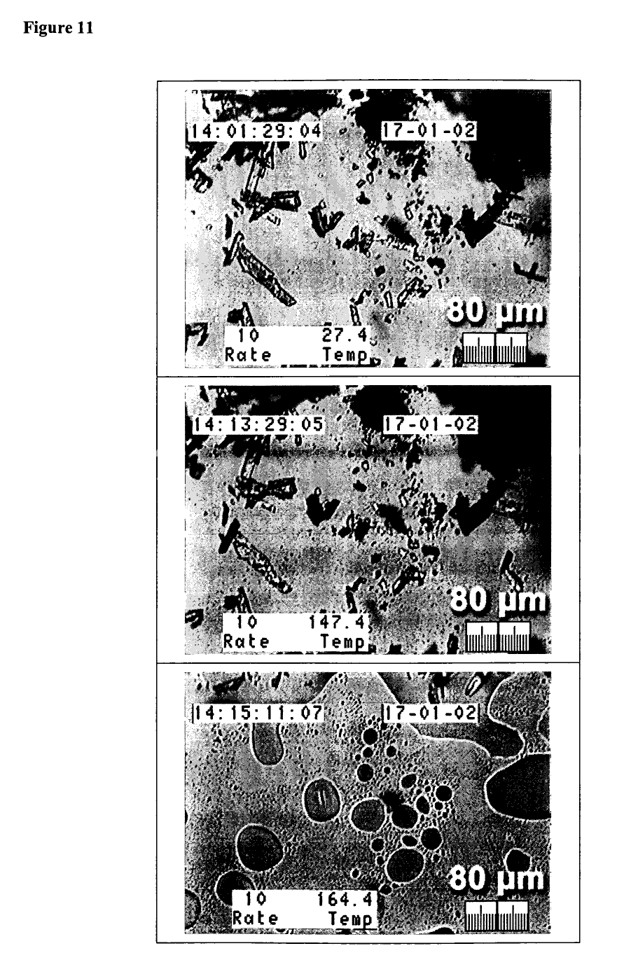

Hotstage microscopy and DSC show that all (S)-amlodipine L-malate lots tested have similar thermal properties. DSC analyses show a single sharp endotherm at 163-165° C. followed by decomposition. Hotstage microscopy shows a single endothermic event at 143-165° C. with no phase changes at lower temperature. See FIGS. 10-11.

8. TGA, Water Content and Moisture Sorption Profiles

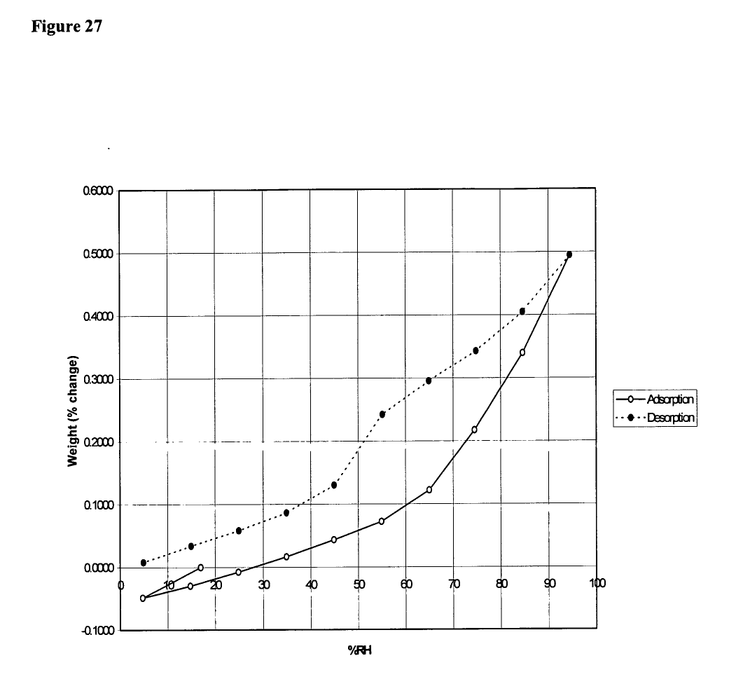

Moisture Sorption Isotherms: Moisture sorption isotherms were generated using VTI SGA-100 symmetric vapor sorption analyzer. Samples were not pre-dried. Equilibrium criteria were the lesser of 0.01 wt % change in 5 minutes or 180 minutes at each RH step. Temperature was fixed at 25° C. and the relative humidity steps (25% to 95% to 25%) were in 5% increments. In FIG. 27, adsorption data is shown as a solid line with desorption data connected by a dashed line. Water Content: Water content was determined by coulometric titration using an EM Science Aquastar® C300 Karl Fischer titrator. TGA: Samples were analyzed using Perkin Elmer TGA 7 thermal gravimetric analyzer at a heating rate of 10° C./min.

TABLE 3
Water Content and Moisture Sorption Data for
(S)-Amlodipine-L-Malate

| | |
|---|---|
| TGA (% wt loss) | 0.14 |
| KF (% H₂O) | 0.13 |
| Moisture Adsorption | 0.24 |
| (% wt gain at 95% RH) | |
| Moisture Desorption | 0.06 |
| (% wt remaining at 25% RH) | |

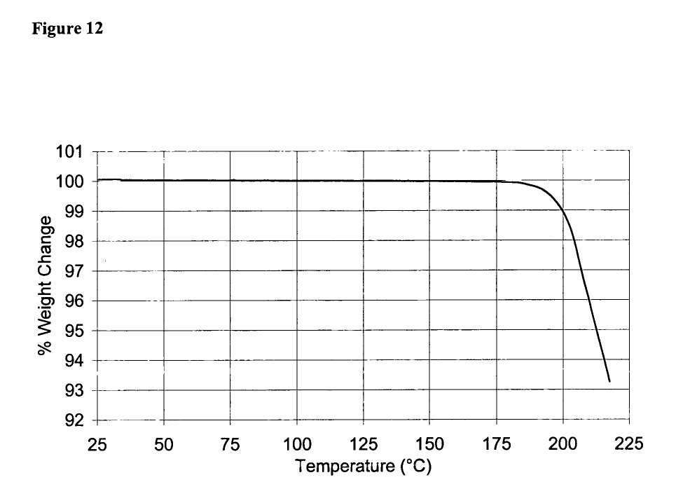

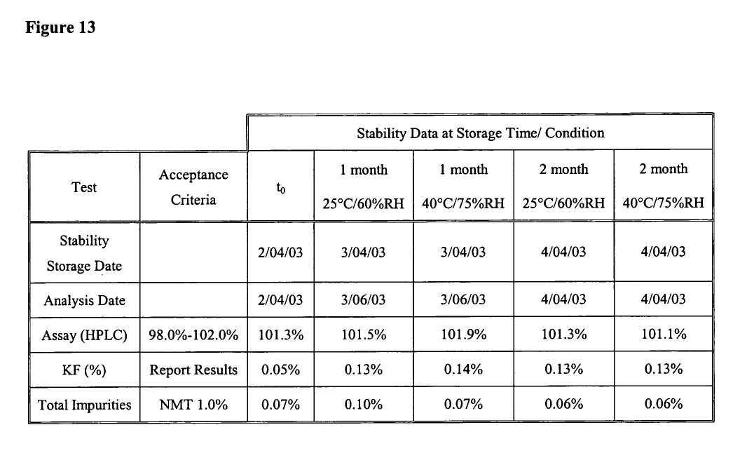

These experiments establish that (S)-Amlodipine L-malate is not hygroscopic with maximum adsorption of 0.30% water at 25° C./95% RH for three lots tested. Sorbed moisture readily desorbed at 25° C./25% RH. TGA and Karl Fischer data confirm that these lots are not hygroscopic. See FIGS. 12 and 13.

9. Solid State Stability

(S)-Amlodipine L-malate API (˜500 mg) was weighed into double polyethylene bags closed with twist ties and placed in small white HDPE containers with snap-on lids. Individual samples were prepared and stored at 25° C./60% RH and 40° C./75% RH. Samples stored at 60° C./75% RH were packaged in uncapped glass vials. All samples were analyzed for assay and impurity by HPLC. See FIGS. 14 and 15.

Solid (S)-amlodipine L-malate is stable for >2 months at 25° C./60% RH and 40° C./75% RH, as shown by KF, HPLC assay and impurity with no degradation observed. Solid (S)-amlodipine L-malate is also fairly stable for >2 months at 60° C./75% RH, as shown by HPLC impurity, with up to 0.2% degradation observed

10. Adhesion Studies

Avicel PH 101 and each (S)-amlodipine salt were pre-screened through a 25-mesh sieve to delump the powders prior to blending. Each (S)-amlodipine salt (0.25 g and 0.75 g) was then mixed with Avicel PH 101 (4.75 g and 2.25 g) for a 5% and 25% API blend, respectively, by manually shaking the mixture in a screw top plastic (HDPE) container for 15 minutes. Approximately 500 mg was weighed into the ½-inch tablet die, a tablet punch was added and 0.2 metric tons of pressure was applied using a Carver press for 2-3 seconds. The tablet was then removed, and the surfaces of the tablet punches (concave, SRC) were washed with mobile phase (1 mL). The wash was then assayed by HPLC. Three measurements were made for each blend.

TABLE 4
Adhesion Data for Various (S)-Amlodipine Salts

| | Adhesion (g (S)-amlodipine/cm²) | |
|---|---|---|
| | 5% (S)-Amlodipine | 25% (S)-Amlodipine |
| (S)-Amlodipine salt | salt in Avicel | salt in Avicel |
| Succinate | 1.6E−06 | 3.3E−06 |
| Maleate | 5.8E−07 | 3.9E−06 |
| (D)-Tartrate | 6.0E−07 | 2.8E−06 |
| (L)-Tartrate | 9.3E−07 | 4.9E−06 |
| (L)-Malate | 4.9E−07 | 2.6E−06 |

The (S)-amlodipine L-malate, when blended at typical levels with a typical excipient, Avicel, showed decreased adhesion to solid dosage form manufacturing equipment compared to blends made with other salts.

11. Polarized Light Microscopy

Samples were analyzed using a Nikon Microphot polarizing light microscope. Samples were prepared in Cargille liquid with a refractive index of 1.600. Samples were imaged with cross-polarized light with a quarter wave plate. See FIG. 15. It was determined that (S)-Amlodipine L-malate contains columnar crystals (length=20-100 μm) with birefringence.

12. X-Ray Powder Diffraction

X-ray powder diffraction analyses, were carried out on a Shimadzu XRD-6000 X-ray powder diffractometer using Cu Kα radiation. The instrument is equipped with a fine-focus X-ray tube and a NaI scintillation detector. The tube voltage and current were set at 40 kV and 40 mA, respectively. Divergence and scattering slits were set at 1°, while the receiving slit was set at 0.15 mm. A theta-two theta region of 2.5 to 40° 2θ was analyzed, using a 3′/min (0.4 sec/0.02° step) continuous scan. Instrument alignment was checked daily using a silicon standard. All the solids formed during the screening experiments were analyzed by XRPD.

13. Surface Area

Specific surface area for (S)-amlodipine L-malate (single measurements) was measured to be 0.7 m²/g using a Micromeritics Gemini 2360 surface-area analyzer. The sample was degassed for 2 hours prior to surface area analysis at 40° C./vacuum using the Micromeritics Vacprep 061. Approximately 0.75 g of salt was placed in a straight-wall sample tube and the analyzer gas was N₂.

The surface area of (S)-amlodipine L-malate SCL was determined using the BET method. BET (Specific) Surface Area measurement was done with a single run (n=1), using 6 different partial pressures of N₂ gas (as the adsorbate). See Brunauer, S., Emmett, P. H., and Teller, E. J. Amer. Chem. Soc. 1938, 60, 309.

14. True, Bulk, and Tapped Density

Bulk and Tap Density: Bulk and tap densities were determined (˜10 grams in a 50 mL graduated cylinder) using a Quantachrome Dual Autotap instrument. The bulk density was measured after 3 taps, and the tapped density was measured after 1000 taps. True Density: True density was determined using a MVP-2 Quantachrome micropycnometer and He gas.

TABLE 5
True, Bulk, and Tapped Density for (S)-Amlodipine-L-Malate

| | |
|---|---|
| Bulk density | 0.31 g/mL |
| Tapped density | 0.49 g/mL |
| True density | 1.38 g/mL |

(S)-Amlodipine L-malate has typical bulk and tapped densities. True density of 1.38 g/cc is typical for a non-porous organic crystalline molecule.

### Example 6

### Procedure for High Performance Liquid Chromatography Assay and Achiral Identification for (S)-Amlodipine-L-Malate

1. Supplies and Equipment - General Reagents*

| | |
|---|---|
| (S)-Amlodipine maleate | Reference Standard |
| Water | HPLC grade (Milli-Q) |
| Acetonitrile | HPLC grade (EM Science) |
| Sodium dihydrogen phosphate, | HPLC grade (JT Baker) |
| monohydrate | |
| (NaH₂PO₄.H₂O) | |
| Triethylamine (TEA) | Reagent grade (B&J Brand) |
| Phosphoric acid, 85% | Reagent grade (JT Baker) |

*Equivalent chemicals may be used as long as system suitability is achieved.

2. Chromatographic Conditions

| | |
|---|---|
| Analytical Column | Zorbax SB-CN, 5 μm, 25 cm × 4.6 mm (Agilent) |
| Mobile Phase | 0.01 M NaH₂PO₄ 0.1% TEA (pH 3.0)/ |
| | Acetonitrile (65:35) |
| Column Temperature | Ambient |
| Flow Rate | 1.0 mL/min |
| Injection Volume | 10 μL |
| Wavelength | 237 nm |
| Run Time | 10 min |

### Example 7

1. Preparation of Amlodipine Maleate Capsules

Capsules were made based on Norvasc® commercial tablet formulation published in the Physician's Desk Reference.

TABLE 6
Preparation of (S)-Amlodipine Maleate Capsules

| | | mg/ | gm/ | Lot # & | |
|---|---|---|---|---|---|
| Drug Excipient | % | Capsule | blend | Grade | Supplier |
| (S)-Amlodipine Maleate* | 0.728 | 3.205 | 4.917 | 1647-03 | Sepracor |
| Dicalcium Phosphate Anhydrous, USP | 73.772 | 324.595 | 497.958 | 3108-USP | C-Rhodia |
| Avicel (Micro Crystalline Cellulose; | 20.000 | 88.000 | 135.000 | 1029-NF | FMC |
| Avicel) PH 101, NF | | | | | |
| Explotab (Sodium Starch Glycolate, | 5.000 | 22.000 | 33.750 | E8857X-NF | Penwest |
| NF; Explotab/Primojel) | | | | | |
| Mg Stearate, NF | 0.500 | 2.200 | 3.375 | 117884-NF | CK-Witco |
| Total | 100.00 | 440.000 | 675.000 | | |
| Swedish Orange Capsule Size # 1 | 1 each | 75.17 | | 585990 | Capsugel |

*1 mg of (S)-Amlodipine is equivalent to 1.282 mg of (S)-Amlodipine Maleate

Note:
a) 2.5 mg of free base equivalent of (S)-Amlodipine maleate required.
b) 5 mg Norvasc tablet disintegrates in luke warm water @<5 seconds.
c) (S)-Amlodipine maleate capsules disintegrate in 10 seconds, 5 seconds for capsule to open.
   2. (S)-Amlodipine Maleate Capsule Development

The stability study began by preparing 1200 capsules. Then, stability experiments were started to test API stability per ICH guidelines. 35 capsules per bottle were used (White HDPE with C/R cap no desiccant). Key for Table 7: X=Assay; H=Hold.

TABLE 7
(S)-Amlodipine Maleate Capsule Development

Time (months)

| Condition | T₀ | T₁ | T₂ | T₃ | T₆ | Tₓ |
|---|---|---|---|---|---|---|
| 25° C./60% RH | X | X | H | X | X | H |
| 30° C./60% RH | n/a | H | H | H | X | H |
| 40° C./75% RH | n/a | X | H | X | H | H |

### Example 8

### Conditions of (S)-Amlodipine Maleate Studies

A suspension in 0.5% CMC. API is unstable at low pH: 1% loss after 5 hours @ pH 1, 1% loss after 50 hours @ pH 5. Note that pH of saturated solution of (S)-amlodipine maleate in water=4.9 (no buffer required).

### Example 9

### Preparation of (S)-Amlodipine-L-Malate Tablets

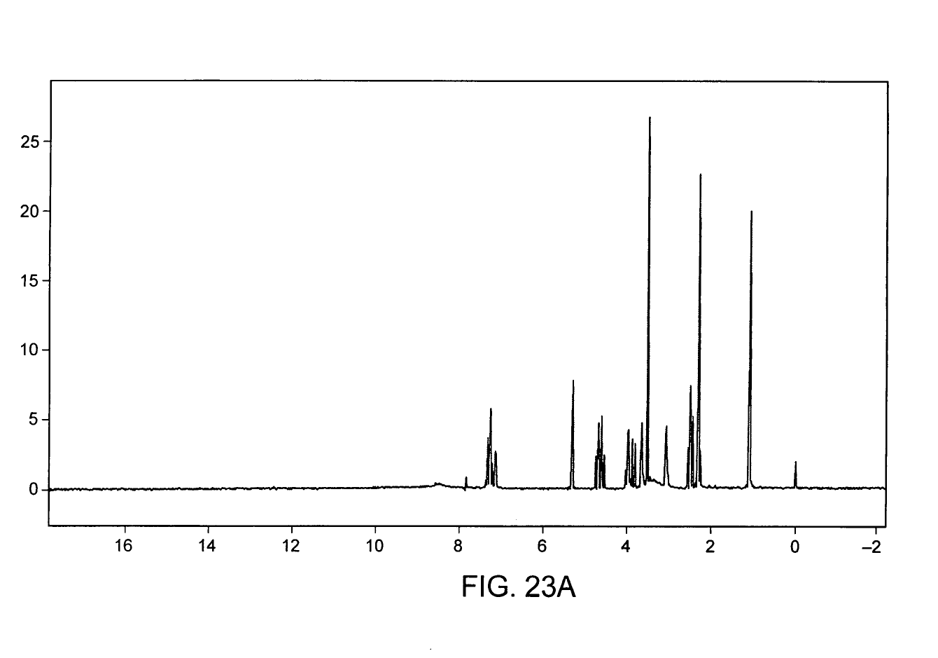

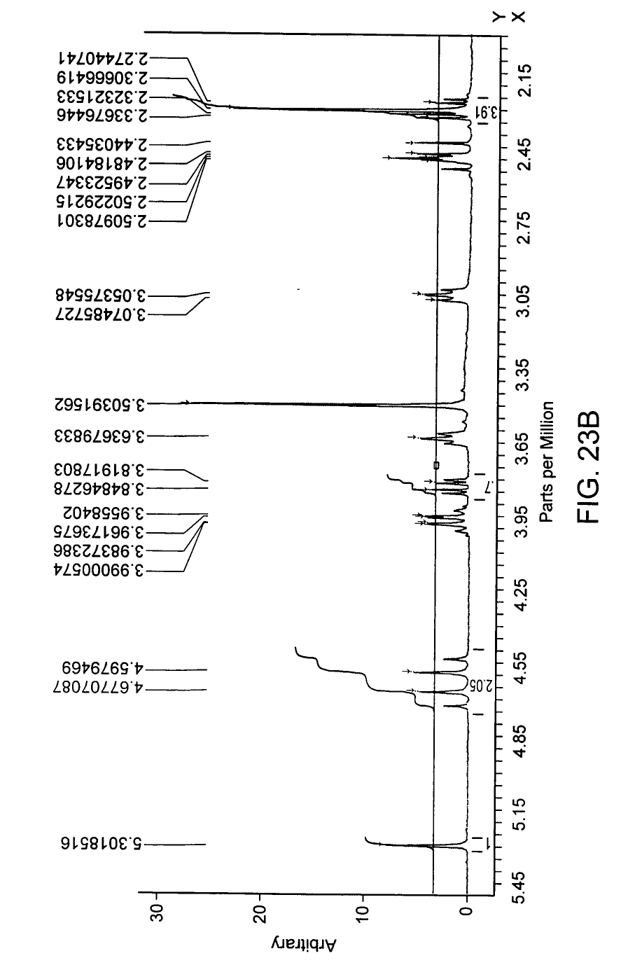

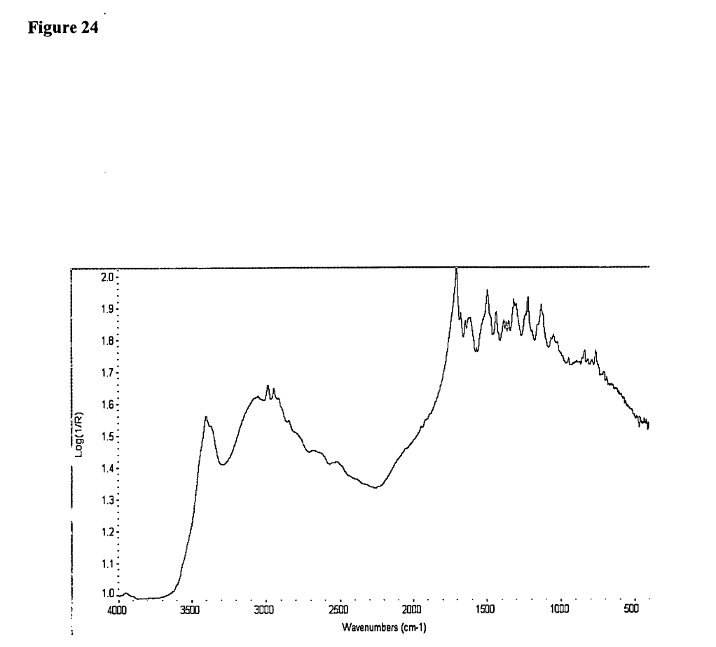

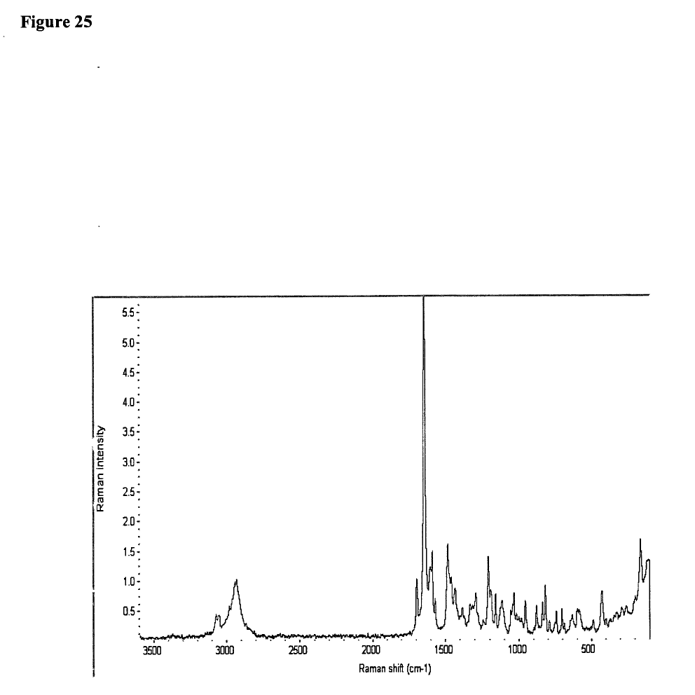

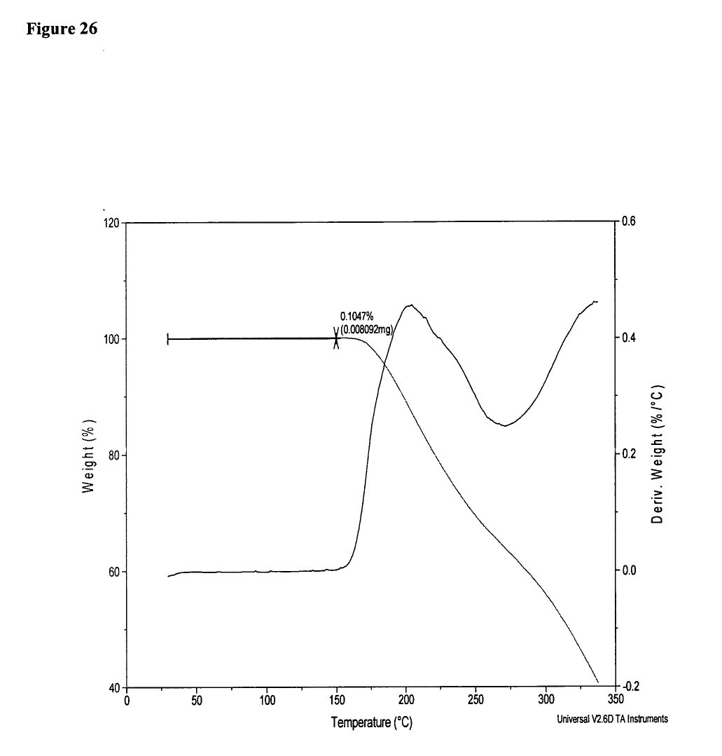

Drug substance and excipients were screened and blended using typical manufacturing equipment. A conventional tablet machine was used to compress this blend into tablets weighing nominally 200 mg each. Tablets were packaged in HDPE bottles with C/R cap with no desiccant, and stored at ICH storage conditions of 25° C./60% RH, 30° C./60% RH, and 40° C./75% RH. Tablets have been assayed at initial time and after 1, 2, 3 and 6 months storage. Stability results are given in FIGS. 23 and 24.

TABLE 8
Preparation of (S)-Amlodipine L-Malate Tablets

| Drug/Excipient | % | mg/Tablet | kg/blend |
|---|---|---|---|
| (S)-Amlodipine-L-Malate* | 3.32 | 6.64 | 0.498 |
| Avicel (Micro Crystalline | 70.7 | 141.36 | 10.602 |
| Cellulose) PH 101, NF | | | |
| Starch 1500 (Pregelatinized | 20.75 | 41.50 | 3.113 |
| Starch), NF | | | |
| Explotab (Sodium Starch | 5.0 | 10.00 | 0.750 |
| Glycolate, NF) | | | |
| Magnesium Stearate, NF | 0.25 | 0.50 | 0.037 |
| Total | 100.0 | 200.0 | 15.000 |

*1 mg of (S)-Amlodipine is equivalent to 1.328 mg of (S)-Amlodipine-L-Malate

### Example 10

### Relative Bioavailability

The pharmacokinetic parameters AUC and Cₘₐₓ from three multiple-dose studies in male and female dogs were compared. The 28-day pharmacokinetic assessments were compared. Male and female dogs were administered an oral capsule once daily containing one of two different salts of (S)-amlodipine. One study used only the maleate salt form of (S)-amlodipine and two studies used only the malate salt form of (S)-amlodipine. All doses were adjusted for salt form so all doses are in terms of mg base/kg.

TABLE 9
Mean Dose-Normalized AUC and Cₘₐₓ Parameters
for (S)-Amlodipine Maleate and Malate Salts Following
28-Days of Oral Capsule Dosing in Dogs

| | Maleate | | Malate | |
|---|---|---|---|---|
| PK Parameter | Male | Female | Male | Female |
| AUC (ng · h/ml)/mg base/kg | 956 | 1146 | 1557 | 1344 |
| Cmax (ng/mL)/mg base/kg | 65.7 | 61.9 | 102 | 89.4 |

The malate salt has also shown greater bioavailability than the maleate salt in both dogs (animal model) and humans. The commercially marketed salt for racemic amlodipine is the besylate and it has been shown that the besylate and maleate have equivalent bioavailabilities.

### Example 11

### Method for Determination of (S)-Amlodipine in Plasma by LC/MS/MS for Preclinical Analysis

An aliquot of each unknown, standard and control sample was analyzed on a high performance liquid chromatographic system equipped with a Positive-Ion mass spectrometer detector (condition tabled below in Table 10).

TABLE 10

| | | |
|---|---|---|
| HPLC | HPLC Column | Chiral-AGP |
| Condition | Mobile Phase | A: 97% 10.0 mM ammonium |
| | | acetate (pH 4.5) |
| | | B: 3% isopropyl alcohol |
| | Separation | Isocratic |
| | Flow Rate | 1.2 mL/minute |
| MS | Source | Turbo ion spray |
| Condition | Source Temp. | 450° C. |
| | Ionization Mode | Positive Ion |

The pharmacokinetic parameters AUC from two multiple-dose studies in people were compared. The 28-day pharmacokinetic assessments were compared. Subjects were administered an oral capsule once daily containing one of two different salts of (S)-amlodipine. One study (I) used only the maleate salt form of (S)-amlodipine and the other study (II) used only the malate salt form of (S)-amlodipine. All doses were adjusted for salt form so all doses are in terms of mg base/kg and AUC measurements were for 0-24 h. These were compared to literature values (III) for single dose studies in which AUC numbers recorded indicate AUC for 0 to infinity (Taken from a paper entitled: Enantioselective disposition of oral amlodipine in healthy volunteers, by Laufen, Heinrich; Leitold, Matyas. Pfizer Mack Res. Dev. Lab., Illertissen, Germany. Chirality 1994, 6(7), 531-6).

TABLE 11

| | | | (S)-Amlodipine dose Normalized AUC | | |
|---|---|---|---|---|---|
| | | | (ng * hr/mL) | | |
| | | | | | Racemic |
| | | Study | (S)-Amlodipine | (S)-Amlodipine | Amlodipine |
| Study | Salt | type | 5 mg | 10 mg | 20 mg |
| I | Maleate | MD | 30.8 | 31.8 | |
| II | Malate | MD | | 55.6 | |
| III | Besylate | SD | | | 35.1# |
| | Maleate | SD | | | 33.0# |

*Normalized to equivalent to (S)-Amlodipine dose = 10 mg
#(S)-Amlodipine levels after single doses of amlodipine besylate salt and each equivalent to 20 mg of amlodipine racemate.

### Example 12

### Method for Determination of (S)-Amlodipine in Human Plasma by LC/MS/MS

An aliquot of each unknown, standard and control sample was analyzed on a high performance liquid chromatographic system equipped with a Positive-Ion mass spectrometer detector (condition tabled below in Table 12).

TABLE 12

| | | |
|---|---|---|
| HPLC | HPLC Column | Chiral-AGP |
| Condition | Mobile Phase | A: 1% 5.0 M ammonium acetate |
| | | (pH 5.5), 1% isopropyl |
| | | alcohol, 0.01% benzylamine in water; |
| | | B: 1% 5.0 M ammonium acetate |
| | | (pH 5.5), 4% isopropyl |
| | | alcohol, 0.01% benzylamine in water |
| | Separation | Gradient |
| | Flow Rate | 400 uL/minute |
| MS | Source | APCI |
| Condition | Source Temp. | 500° C. |
| | Ionization Mode | Positive Ion |

### Example 13

### Polymorph and Solvate Analysis of (S)-Amlodipine Malate

(S)-Amlodipine L-malate has several polymorphic and solvated forms. They were formed through crystallization and mechanical techniques. Characterization of crystal forms produced during the screen described below were performed using X-ray powder diffraction (XRPD), differential scanning calorimetry (DSC), thermogravimetry (TG), hot stage microscopy, moisture balance, solution proton NMR spectroscopy, thermogravimetry-infrared spectroscopy (TG-IR), infrared (IR) and Raman spectroscopy.

1. Polymorph-Solvate Screen and Characterization

Polymorph and solvate screen samples were generated by weighing a sample of form A of (S)-amlodipine L-malate (approximately 30 mg) and adding a given volume of test solvent (between 1-20 mL). The samples were then heated on a shaker block (approximately 60° C.) for thirty minutes; the solution was filtered, then left in an open vial under ambient conditions (fast evaporation). The procedure was then repeated and the resulting solution was left under ambient conditions in a vial covered with a lid containing pinholes (slow evaporation).

The same procedure was repeated at elevated temperature (approximately 60° C.) by keeping the mixture on a hot plate at the desired temperature. The resulting solution was rapidly filtered into a vial kept on the same hot plate. The heat source was turned off and the hot plate and vial were allowed to cool to ambient temperature (slow cools). The vial was then placed in a freezer. In some cases, the warm vials were placed directly in the freezer from the hotplate (fast cools). Solids were removed by filtration or decantation and allowed to dry in the air.

Crystallization using anti-solvents were also performed (crash crystallizations). Solid was dissolved in a solvent and filtered into anti-solvent cooled in a dry ice/acetone bath. Samples were then placed in a refrigerator and freezer to further enhance crystallization. Solids were removed by filtration or decantation and allowed to dry in the air. Vapor diffusion chambers were generated by dissolving solid in a solvent and then filtering to remove seeds. This solution was then placed in an open faced vial which was then enclosed in a chamber containing a volatile anti-solvent.

Slurry studies were performed by saturating a given volume of solvent (approximately 3 mL) with form A of (S)-amlodipine L-malate and allowed the sample to sit on an orbital shaker at room temperature or 40° C. for approximately one week.

Centrifuge evaporator crystallization experiments were performed. Solid was dissolved in a solvent and then the vial was placed into a centrifuge that was attached to a vacuum pump/cold trap, which stripped the solvent under vacuum.

A portion of form A of (S)-amlodipine L-malate was ground in a Wig-L-Bug amalgamator for 15, 30, 45 and 60 minutes. The samples were then analyzed using XRPD. A portion of (S)-amlodipine L-malate was also lightly ground with a mortar and pestle for approximately one minute. A portion of lot was ground in a Spex Certiprep model 6750 Freezer Mill. In this experiment, the sample was milled while submerged in liquid nitrogen. The sample was milled for six cycles, where one cycle of cryogenic milling is defined as three, two minute, milling cycles with two minute cooling intervals between milling cycles.

Hygroscopicity studies were performed by placing portions of a select form in a 11, 32, 45, 66, 75, 84% or 95% relative humidity (RH) chamber for approximately one to three weeks. Desolvation studies were carried out by heating each form in an 80° C. oven for approximately one week. Interconversion experiments were carried out by making slurries containing two forms in a saturated solvent. The slurries were agitated for approximately seven days at ambient temperature. The insoluble solids were recovered by filtration and analyzed using XRPD.

A. X-ray Powder Diffraction

X-ray powder diffraction analyses were carried out on a Shimadzu XRD-6000 X-ray powder diffractometer using Cu Kα radiation. The instrument is equipped with a fine-focus X-ray tube. The tube voltage and amperage were set at 40 kV and 40 mA, respectively. The divergence and scattering slits were set at 1° and the receiving slit was set at 0.15 mm. Diffracted radiation was detected by a NaI scintillation detector. A theta-two theta continuous scan at 3′/min (0.4 sec/0.02° step) from 2.5° 20 to 40° 20 was used. A silicon standard was analyzed each day to check the instrument alignment. Each sample was prepared for analysis by pressing it onto a sample holder. For variable temperature (VT-XRPD) runs, the powder patterns were collected at elevated temperatures ranging from 25-150° C. The sample was maintained at each temperature for 1 minute.

Other X-ray powder diffraction analyses were carried out using Cu-Kα radiation on an Inel XRG-3000 diffractometer equipped with a curved position-sensitive detector. Data were collected in real time over a 2 theta range of 120° at a resolution of 0.03°. The tube voltage and current were 40 kV and 30 mA, respectively. Samples were packed in an aluminum holder with a silicon insert and analyzed. A silicon standard was analyzed each day to check for instrument alignment. Only the region between 4 to 40° 2 [symbol] are shown for data run on this instrument.

B. Thermal Analyses

TG analyses were carried out on a TA Instrument TGA 2050. The calibration standards were nickel and Alumel™. Approximately 10 mg of sample was placed in a tared platinum or aluminum pan, accurately weighed, and inserted into the TG furnace. The samples were heated at a rate of 10° C./min., to a final temperature of 300 or 350° C. under flow of N₂.

DSC data were obtained on a TA 2920 instrument. The calibration standard was indium. A sample approximately 3-5 mg in weight was placed into a tared DSC pan, and the weight accurately recorded. Hermetically sealed pans with one pinhole or open pans were used for analysis and the samples were heated under nitrogen at a rate of 1 or 10° C./min, up to a final temperature of 350° C.

Modulated differential scanning calorimetry (MDSC) data were obtained on a TA Instruments differential scanning calorimeter 2920 equipped with a refrigerated cooling system (RCS). The sample was placed into an aluminum DSC pan, and the weight accurately recorded. The pan was covered with a lid. MDSC data were obtained using a modulation amplitude of +/−0.8° C. and a 60 second period with an underlying heating rate of 1° C./min from −10-120° C., or from 0-220° C. The temperature and the heat capacity were calibrated using indium metal and sapphire as the calibration standards, respectively.

Hot-stage microscopy was carried out using a Kofler hot stage mounted on a Leica Microscope. The instrument was calibrated using USP standards.

A TA Instruments TGA 2050 interfaced with a Nicolet model 560 Fourier transform IR spectrophotometer, equipped with a globar source, Ge/KBr beamsplitter, and deuterated triglycine sulfate (DTGS) detector, was utilized for TG-IR experiments. The IR spectrometer was wavelength calibrated with polystyrene on the day of use, while the TG was temperature and weight calibrated weekly, using nickel and Alumel™ for the temperature calibration. A sample of approximately 3-9 mg of (S)-amlodipine L-malate was weighed into a sample holder, then heated at a rate of 20° C./min with a helium purge. IR spectra were obtained in series, with each spectrum representing 32 co-added scans at a resolution of 4 cm⁻¹. Spectra were collected with a 17 second repeat time. TG/IR analysis data are presented as Gram-Schmidt plots and IR spectra linked to the time. Gram-Schmidt plots show total IR intensity vs. time, hence the volatiles can be identified at each time point. They also show when the volatiles are detected. From the Gram-Schmidt plots, time points are selected and the IR spectra of these time points are presented in the stacked linked spectra. Each spectrum identifies volatiles evolving at that time point. Volatiles were identified from a search of the HR Nicolet TGA vapor phase spectral library. The library match results are also presented to show the identified vapor.

C. Spectroscopy

Raman spectra were acquired on a Fourier transform Raman accessory interfaced with a Nicolet model 860 IR bench utilizing an excitation wavelength of 1064 cm⁻¹ and approximately 0.5 W of Nd:YAG laser power. The Raman spectra were measured with an indium gallium arsenide (InGaAs) detector. The spectra represent 256 co-added scans acquired at 4 cm⁻¹ resolution. The spectrometer was calibrated (wavelength) with sulfur and cyclohexane at the time of use.

The mid-IR spectra were acquired on a Nicolet model 860 Fourier transform IR spectrophotometer equipped with a globar source, Ge/KBr beamsplitter, and deuterated triglycine sulfate (DTGS) detector. A Spectra-Tech, Inc. diffuse reflectance accessory was utilized for sampling. Each spectrum represents 256 co-added scans at a spectral resolution of 4 cm−1. A background data set was acquired with an alignment mirror in place. A single beam sample data set was then acquired. Subsequently, a Log 1/R(R=reflectance) spectrum was acquired by ratioing the two data sets against each other. The spectrophotometer was calibrated (wavelength) with polystyrene at the time of use.

D. Moisture Balance

Moisture-sorption data were collected on a VTI SGA-100 moisture balance system. For sorption isotherms, a sorption range of 5% to 95% relative humidity (RH) in 10% RH increments was used for analysis. The sample was not dried prior to analysis. Equilibrium criteria used for analysis were less than 0.0100 weight-percent change in 5 minutes with a maximum equilibration time of 3 hours if the weight criterion was not met. The data were not corrected for the initial moisture content of the sample.

E. Solution Proton NMR

NMR spectra for each form were obtained on a 250 MHz spectrometer. Samples were dissolved in DMSO-d6. Instrumental parameters include a frequency of approximately 250 MHz, pulse width of 4.0 microseconds, and a relaxation delay of 5.0 seconds.

1. (S)-Amlodipine-L-Malate (Form A)

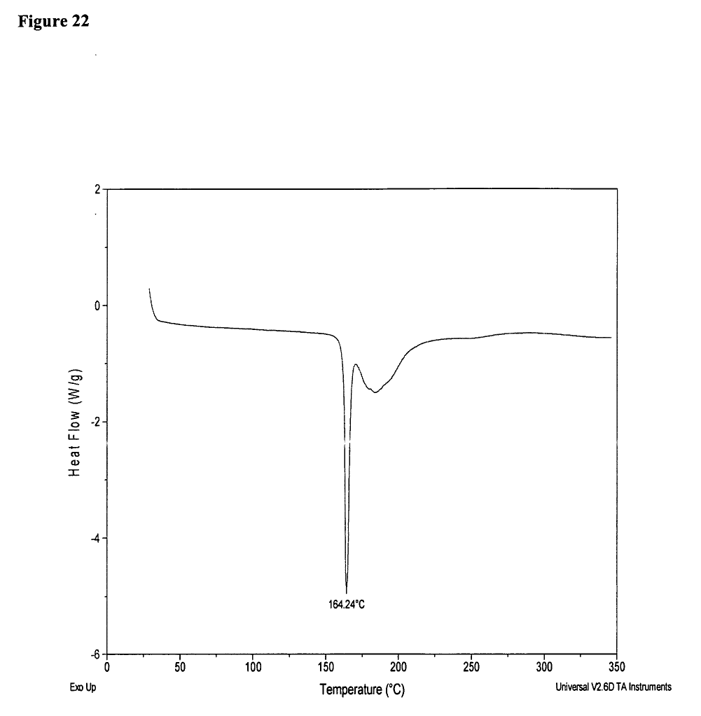

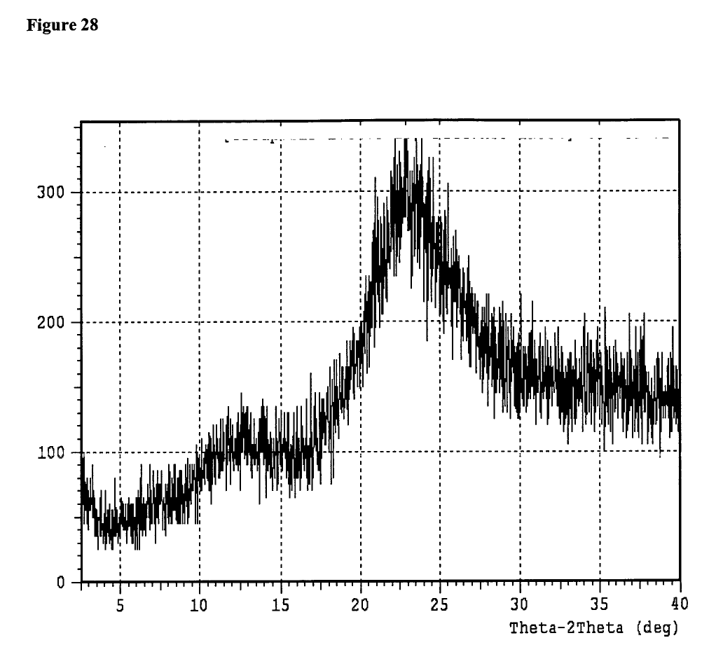

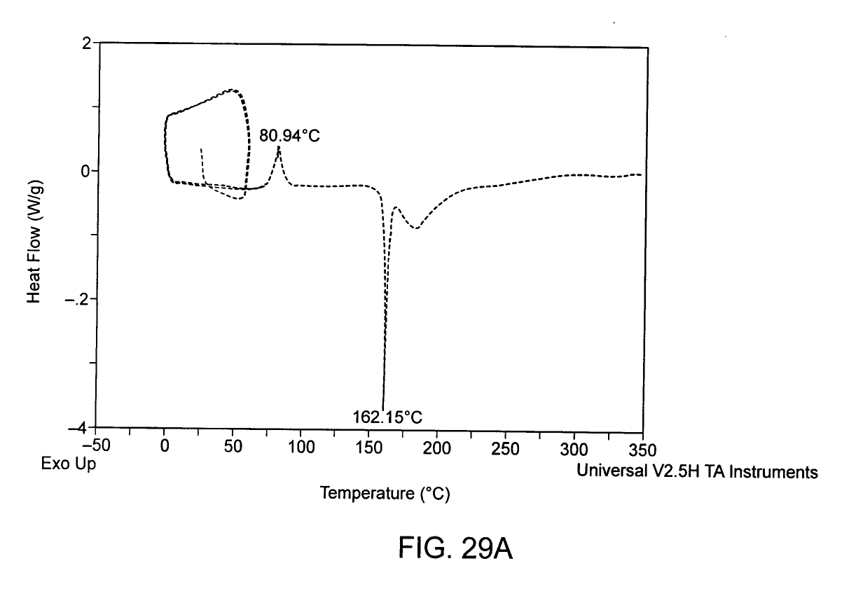

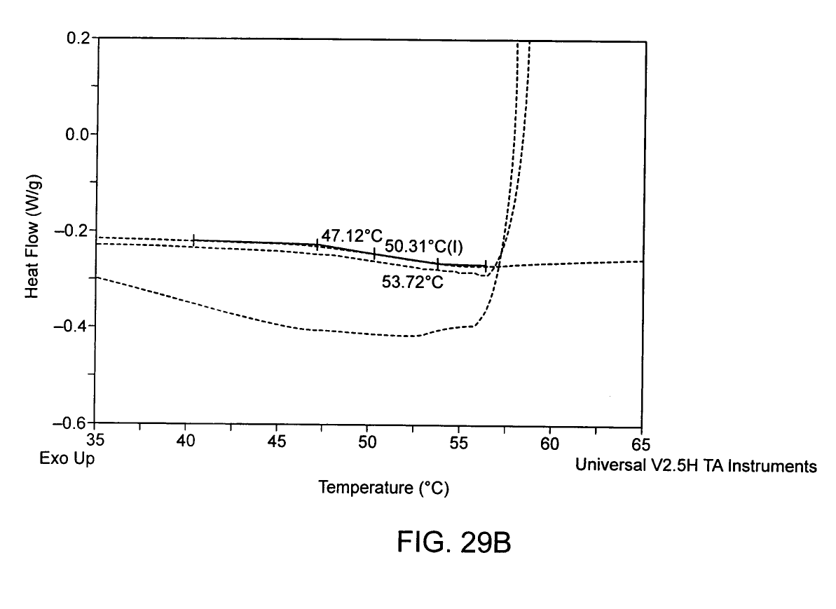

Form A was found to lose approximately 0.1% up to 150° C. indicating an unsolvated material. The DSC curve (FIG. 22) for form A shows an endotherm at 164° C. This was attributed to a melt based on hotstage data. Moisture balance data is shown in FIG. 27. Form A showed an increase in weight of 0.5% when equilibrated at 95% RH. The sample then lost this weight upon equilibrating back to 5% RH. XRPD data collected on the sample after the moisture balance experiment indicated that the sample form remained unchanged. Hygroscopicity studies shown that upon equilibrium at 31, 75, 84 and 95% relative humidity for approximately one week, form A remained unchanged. Solution ¹H NMR data indicated that the (S)-amlodipine L-malate molecule was intact (FIG. 23). IR and Raman spectra were collected and are plotted in FIGS. 28 and 29. Based on these studies Form A is a crystalline, non-solvated material, which melts at 162° C.

2. Amorphous (S)-Amlodipine-L-Malate

An amorphous material was generated by both room temperature milling and cryogenic milling. At ambient temperature, amorphous material was produced by grinding in a mixer mill for a total of 40 or 50 minutes in 10 minutes intervals. (The sample was scraped from the walls of the canister every ten minutes). The 40 minute grind was performed at 30 Hz. Ambient temperature grinding was also performed in an amalgamater for 30 and 45 minutes in 15 minute intervals. A cryogrinder was also used to make amorphous material. The sample was ground under liquid nitrogen for 6 cycles, where a cycle=3×2-minute grinding times with two minutes of cooling between grinds.

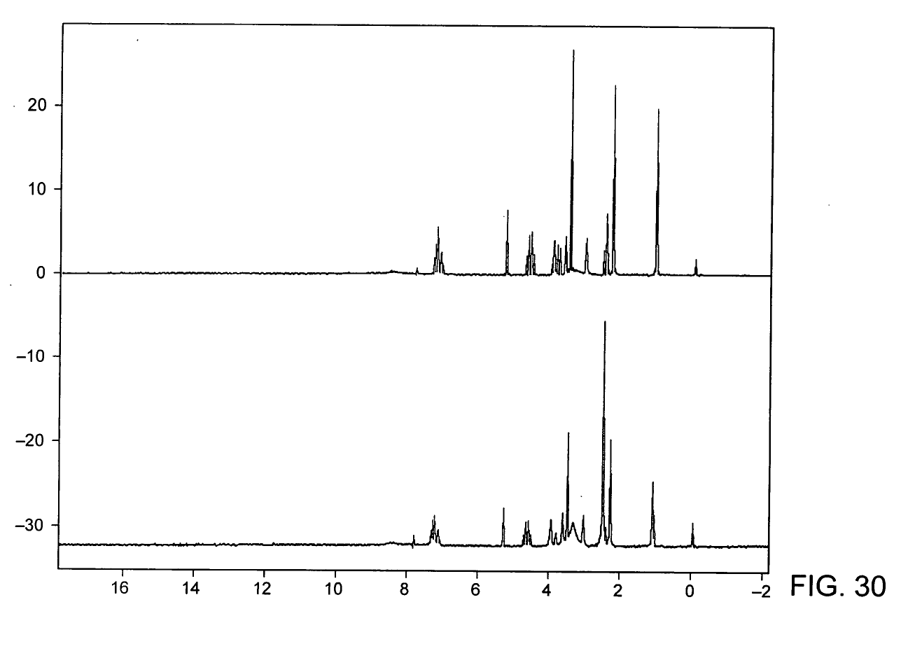

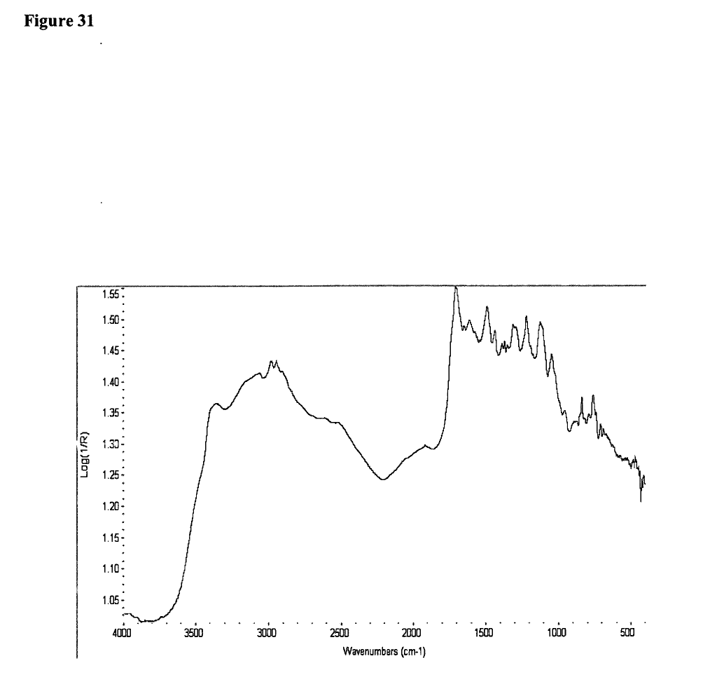

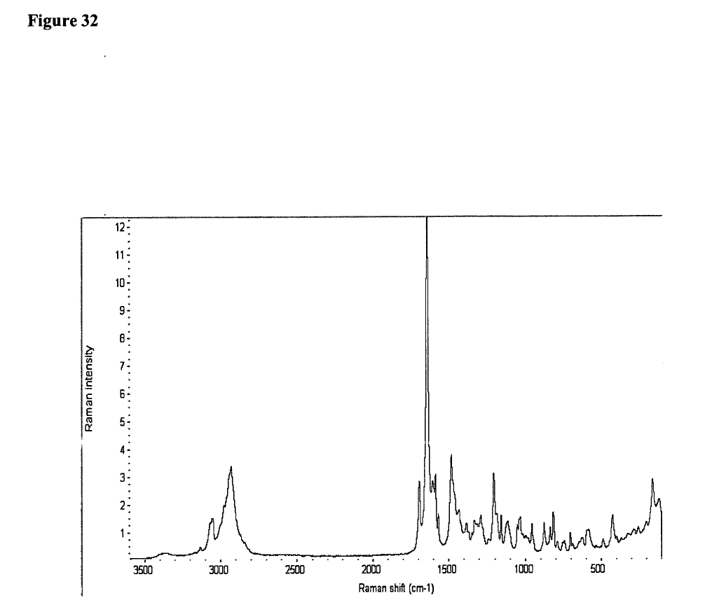

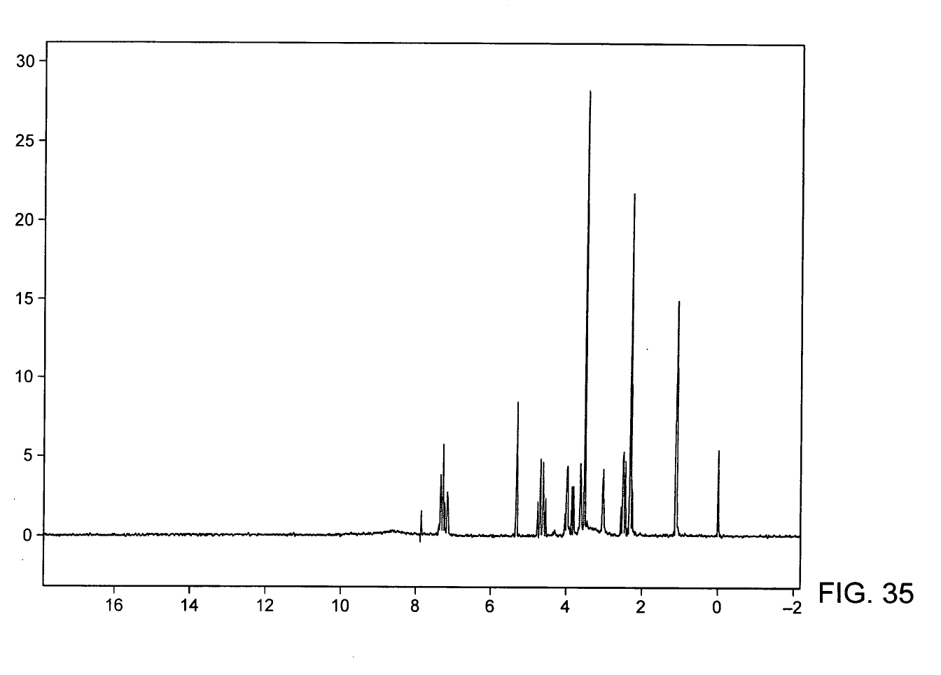

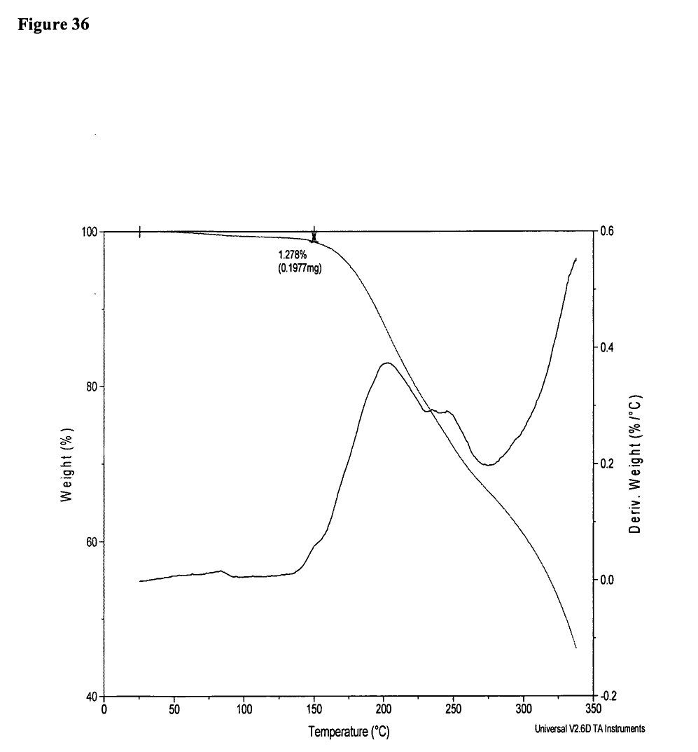

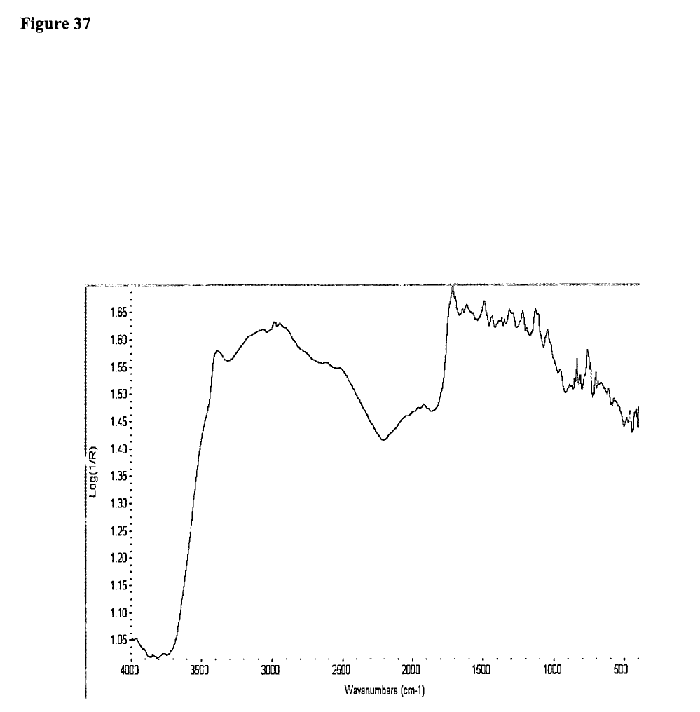

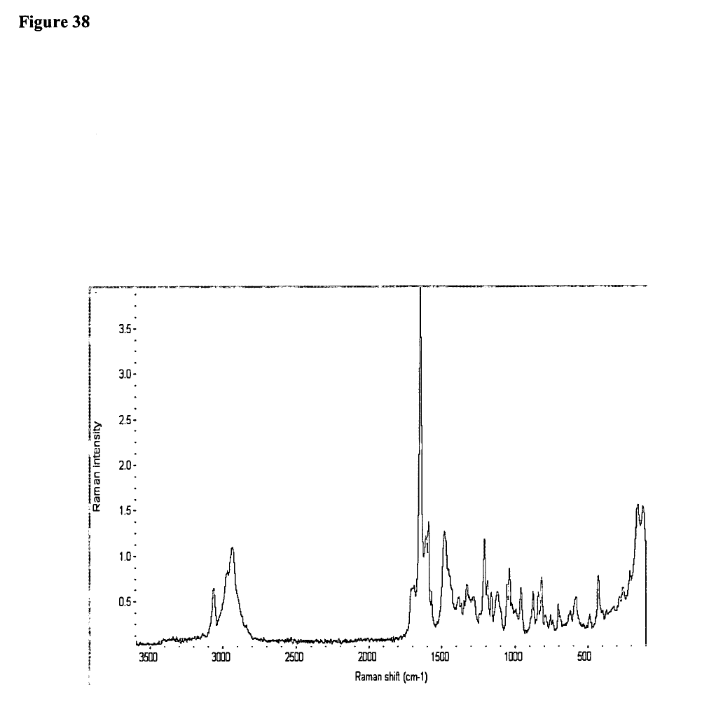

A representative XRPD pattern is shown in FIG. 28. Solution ¹H NMR data indicated that the (S)-amlodipine L-malate molecule was intact (FIG. 30). IR and Raman spectra were collected and are plotted in FIGS. 35 and 36. The IR and Raman spectra of the amorphous form are virtually identical to those for form A (FIGS. 28 and 29). The DSC curve for the amorphous form (FIG. 29) shows an exotherm at 81° C. and an endotherm at 162° C. This may be due to the crystallization to form A followed by the form A melt. A glass transition was measured around 54° C.

3. (S)-Amlodipine-L-Malate Hydrate (Form B)

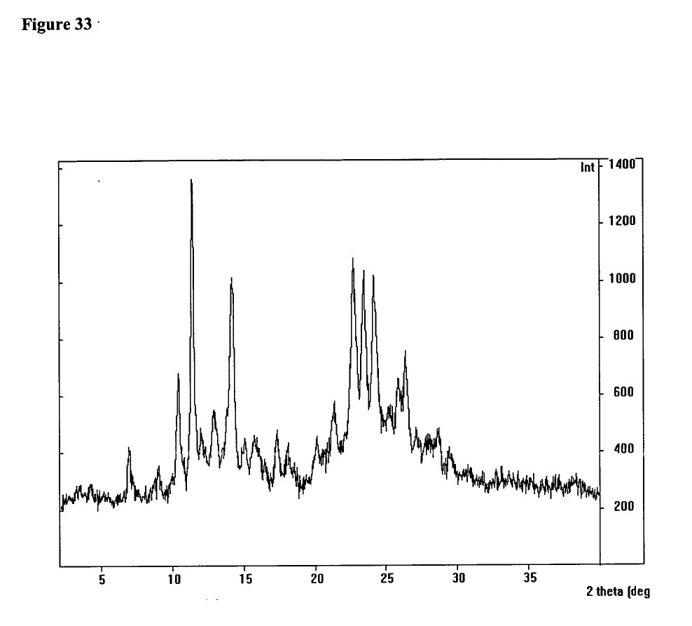

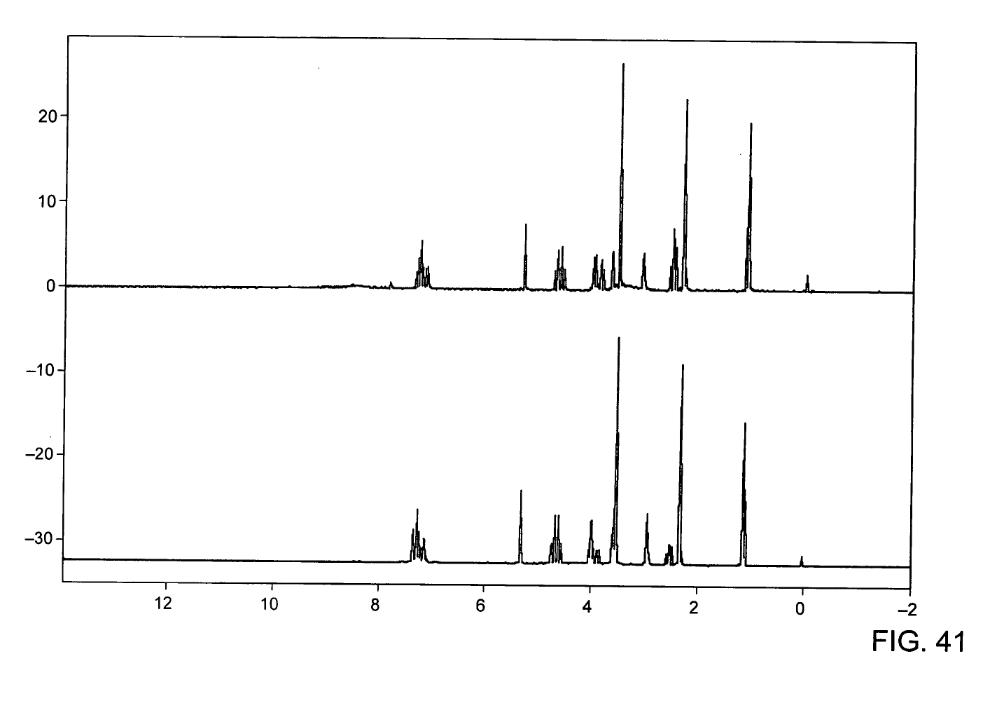

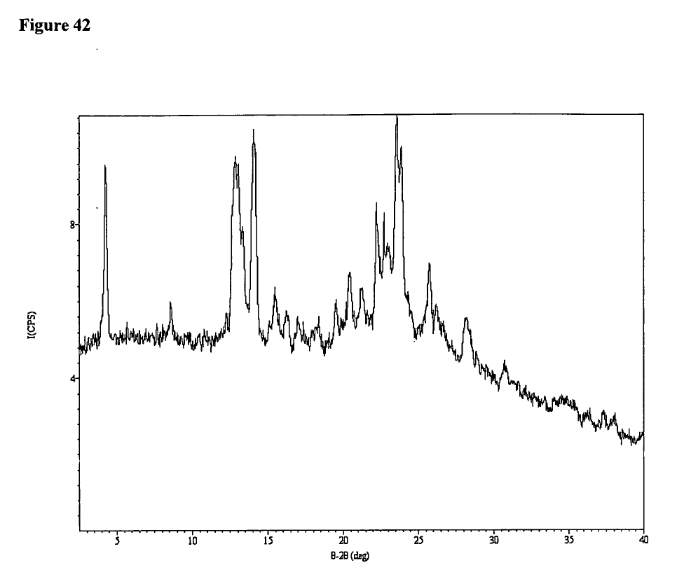

Form B was obtained from water evaporation, slow evaporation from dioxane, fast and slow evaporations from EtOH, and a fast evaporation from IPA. A XRPD representative pattern is shown in FIG. 33. Solution ¹H NMR indicated that the (S)-amlodipine molecule was intact (FIG. 35). IR and Raman spectra were collected and are plotted in FIGS. 41 and 42. Compared to form A, the IR and Raman for form B are virtually identical.

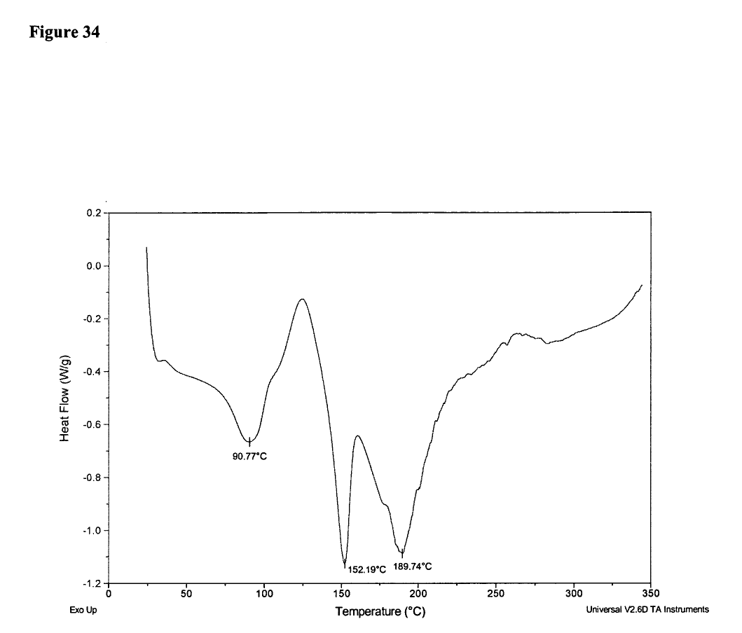

The DSC curve for form B (FIG. 34) shows endotherms at ˜91, ˜152, and ˜190° C. The endotherm at ˜152° C. was attributed to the melt based on hotstage, while events correlated to thermal activity around 91 and 190° C. in the DSC curve were not observed during the hotstage investigation. Variable temperature XRPD experiments were performed on form B. The XRPD data suggests that around 100° C., form B begins to undergo a conversion because the XRPD pattern is mostly amorphous. Furthermore, by 125° C., the sample displayed an XRPD pattern indicative of form A.

Desolvation studies were performed on form B. When form B was heated at approximately 60° C. for approximately one week, it remained unchanged. When form B was placed in an approximately ˜0% relative humidity chamber, the form remained unchanged. Moisture balance data showed an increase in weight of 17.2% when equilibrated at 95% RH. The sample then lost this weight upon equilibrating back to 5% RH.

Form B was found to lose 1.3% volatiles up to 150° C. Karl Fischer water analysis esulted in 4.75% water. TG-IR analysis confirmed the Karl Fischer water analysis. Form B appears to be a hydrate because is was predominately crystallized from experiments involving water and the Karl Fischer data (1.5 moles of water) suggests more water than what can be attributed to just surface water. Form B was also crystallized from dioxane, IPA and EtOH without the presence of water, however these solvents may have contained water sorbed from the atmosphere. Form B appears to be a hydrate.

4. (S)-Amlodipine-Hemi-L-Malate (Form C)

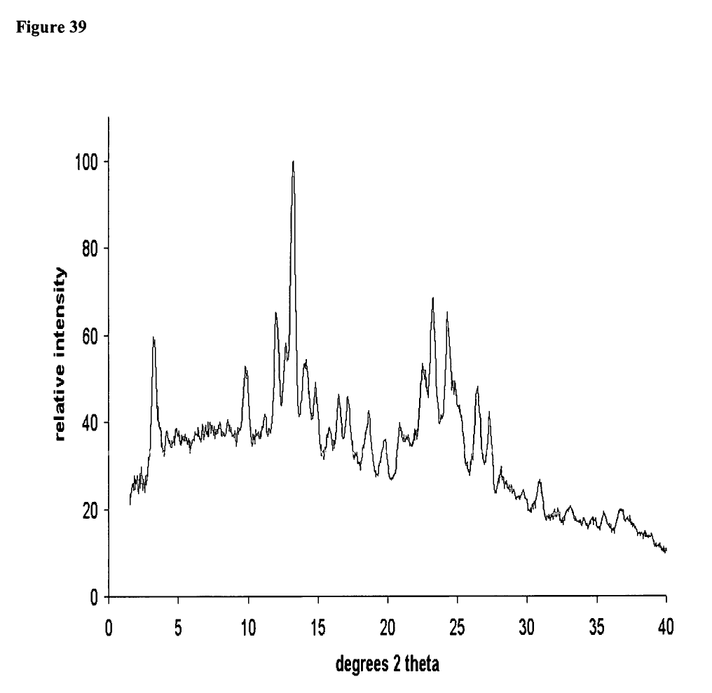

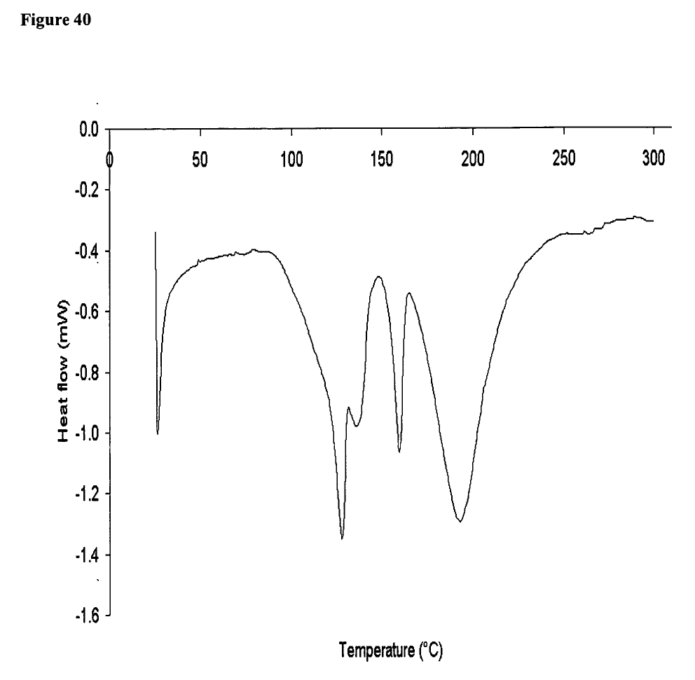

Form C slurried from water, 1:4 EtOH:water, and 1:4 MeOH:water. A representative XRPD pattern is shown in FIG. 39. The characterization of these samples via solution ¹H NMR show that form C is the hemi-salt of (S)-amlodipine L-malate (i.e., a salt consisting of 2 molecules of amlodipine for every molecule of L-malic acid; FIG. 40). The hemi-malate could also be made from mixing two equivalents of (S)-amlodipine with one equivalent of L-malic acid in ethanol.

5. (S)-Amlodipine-L-Malate (Form D)

Form D was obtained from crystalization from ethanol: ethanol (2 mL) was added to (S)-amlodipine L-malate (68.4 mg). The sample was sonicated and then placed on a 60° C. shaker block. All solids had dissolved after approximately one day at 60° C. The sample was then plunged into a dry ice/acetone bath and then placed in a freezer. After approximately five months, the solvent was decanted, and the solids were allowed to air dry.

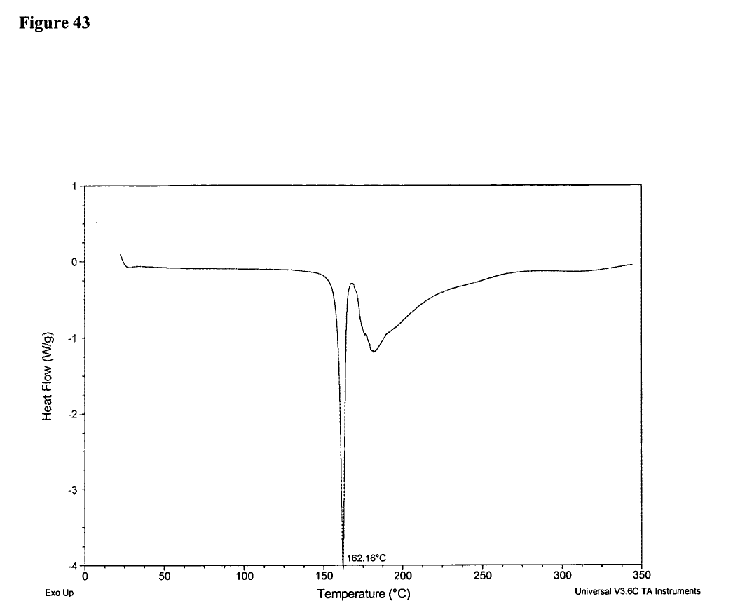

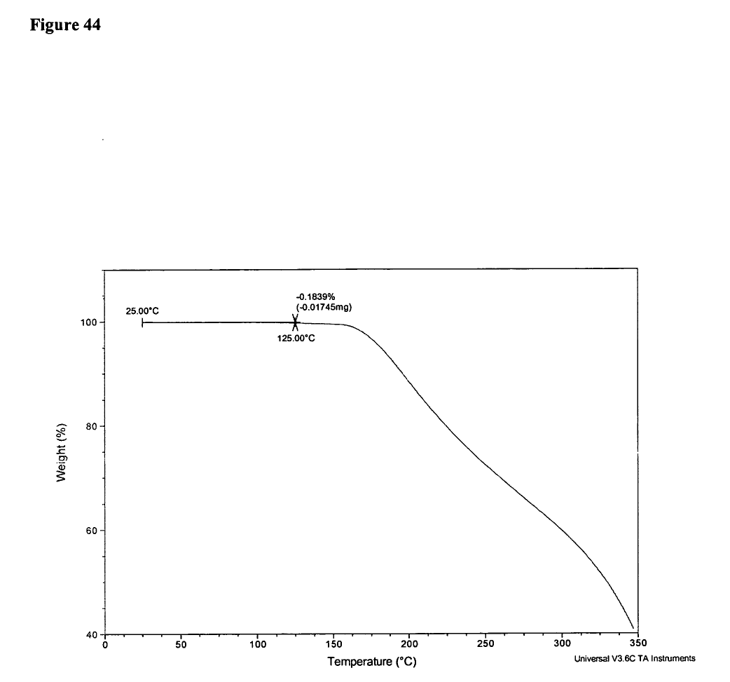

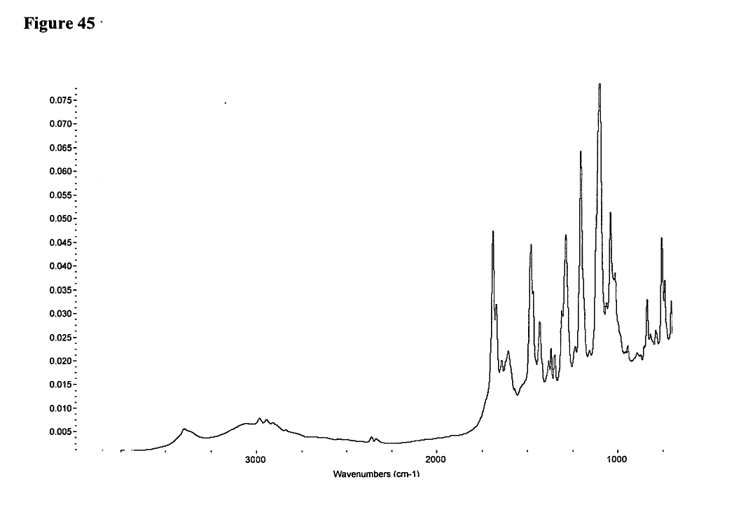

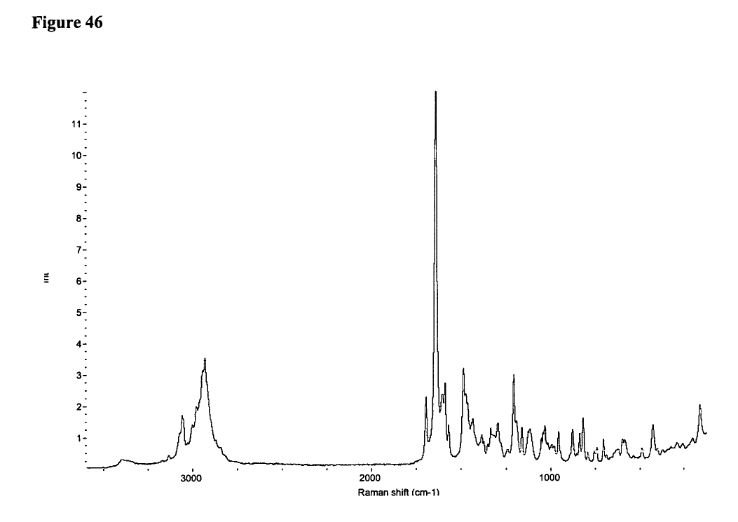

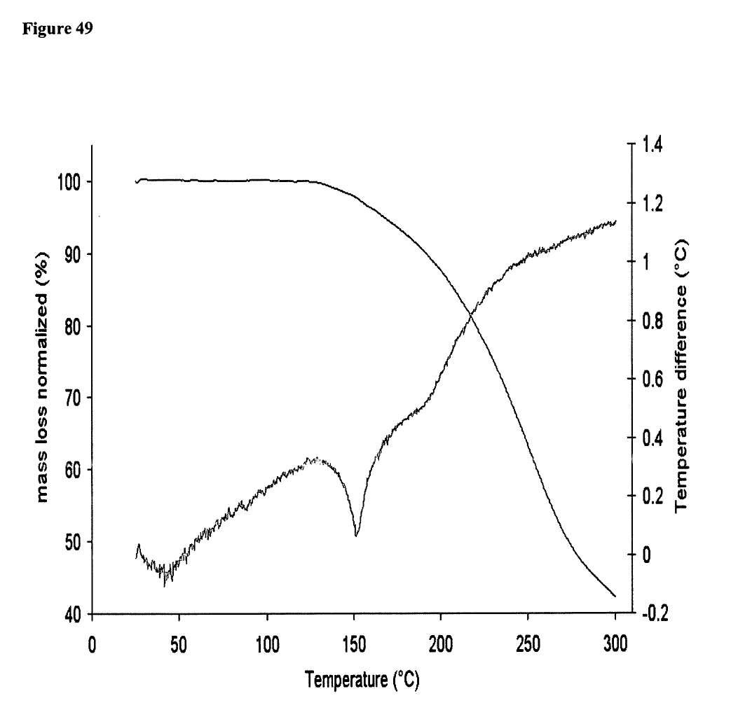

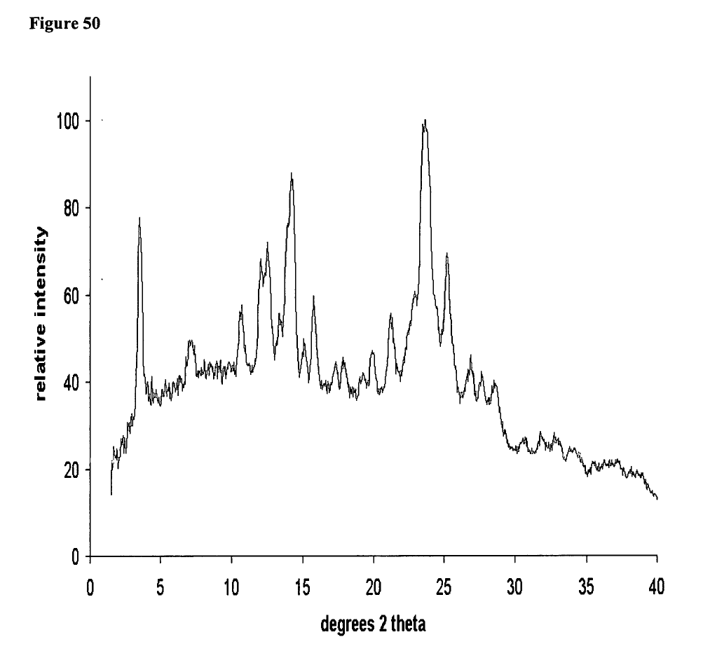

A representative XRPD pattern is shown in FIG. 42. The DSC curve for the D form (FIG. 43) shows an endotherm at 162° C. The TGA spectra (FIG. 44) shows 0.2% weight loss at 125° C. IR and Raman spectra are shown in FIGS. 49 and 50. Moisture balance experiments showed a 1.5% weight increase from 5% to 95% RH and a return to initial weight upon desorption.

### Example 14

### Second Polymorph and Solvate Analysis of (S)-Amlodipine Malate

(S)-Amlodipine L-malate has several polymorphic and solvated forms. They can be formed through crystallization and mechanical techniques. Characterization of crystal forms produced during the screen described below were performed using X-ray powder diffraction (XRPD), differential scanning calorimetry (DSC), thermogravimetry (TG), hot stage microscopy, moisture balance, solution proton NMR spectroscopy, thermogravimetry-infrared spectroscopy (TG-IR), infrared (IR) and Raman spectroscopy.

1. Polymorph-Solvate Screen and Characterization

A primary polymorph-solvate screen of different crystallization conditions was performed. Plates were divided into two parts in which each part contained a different concentration of starting material in solvent: 65 and 130 mg/mL. A stock solution was prepared by dissolving 6.5 g of (S)-amlodipine L-malate in 100 mL methanol. The wells were dosed in 8 stages, the low concentration wells were dosed in 8 stages of 45 μL and the high concentration wells were dosed in 8 stages of 90 μL. The plates were placed in a vacuum chamber at full vacuum (<5 kPa) at room temperature for 1 day between each dosing stage. After the stock solvent was evaporated the well plates were charged with different solvents and each well individually sealed.

The plates containing (S)-amlodipine L-malate and crystallization solvents were subjected to a series of temperature profiles: from room temperature, the plates were heated to an initial temperature of 60° C. at a rate of 4.8° C./min and, after 30 minutes, cooled at a slow (1° C./h), medium (5° C./h) or fast (30° C./h) rate to a final temperature of 5 or 25° C. and held at that temperature for 72 h. After the temperature profile, the plates were uncovered and placed in a vacuum chamber at full vacuum (<5 kPa) at room temperature until the solvents evaporated and the crystals “appeared” dry. An extra plate was prepared in which the wells containing high boiling solvents were kept under vacuum for longer period of time, in order to remove these solvents completely.

The crystallization experiments were carried out in stainless steel (316 L) well plates. The plates contain 96 individually sealed wells of 50 μL total volume. After crystallization and solvent evaporation the crystalline products were harvested and measured using Crystallics' T2 high throughput XRPD set-up. The plates were mounted on a Bruker GADDS diffractometer that is equipped with a Hi-Star area detector. The data collection was carried out at room temperature using the monochromated CuK_{α} radiation in the region of 20 between 3 and 42°. The diffraction pattern of each well was collected in two 2theta ranges (1.5≦2θ≦19.5° for the 1st frame, and 21.5≦2θ≦41.5° for the second frame) with an exposure time of 90 s for each frame.

After identification of the various solid forms thermal analysis was used for further characterization. Melting properties were obtained from differential scanning calorimetry (DSC) thermograms recorded with a DSC822e (Mettler-Toledo GmbH, Schwerzenbach, Switzerland). The DSC822e was calibrated for temperature and enthalpy with a small piece of indium (mp=156.6° C.; ΔH_{f}=28.45 J g⁻¹). Samples were sealed in standard 40 μL aluminium pierced pans and heated in the DSC from 25 to 300° C. with a heating rate of 20° C. min⁻¹. Dry N₂ gas was used to purge the DSC equipment during measurement at a flow rate of 50 mL min⁻¹. According to convention exothermic events are plotted upwards and endothermic events downwards.

Mass loss due to solvent or water efflorescence was determined by thermogravimetric analysis (TGA). During heating of a sample in a TGA/SDTA851e (Mettler-Toledo GmbH, Schwerzenbach, Switzerland) the weight of the sample was monitored resulting in a weight vs. temperature curve. The TGA/SDTA851e was calibrated for temperature with indium and aluminium. Samples were weighed in 40 or 100 μL aluminium crucibles and heated in the TGA from 25 to 300° C. with a heating rate of 20° C. min⁻¹. Dry N₂ gas was used to purge the equipment at a rate of 80 mL min⁻¹.

2. (S)-Amlodipine-L-Malate Solvate (Form E)

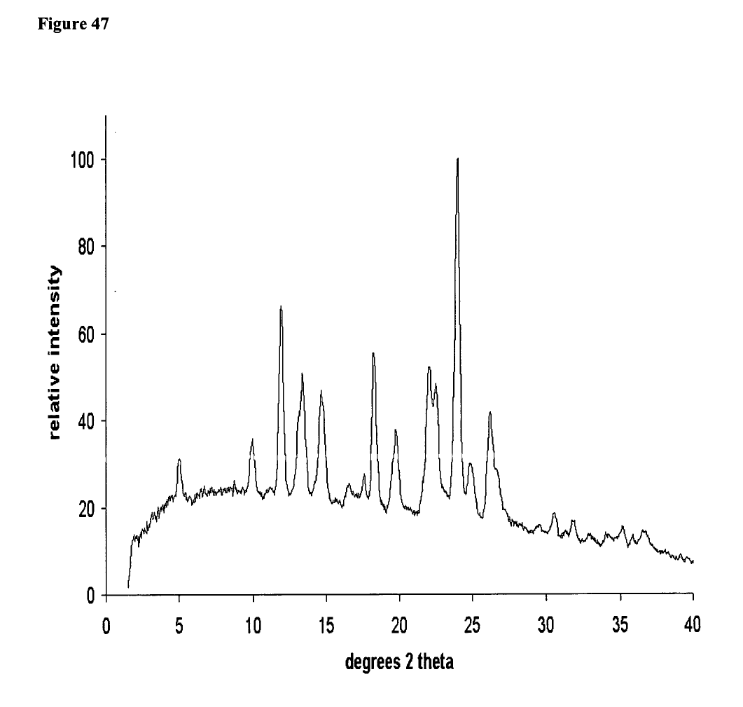

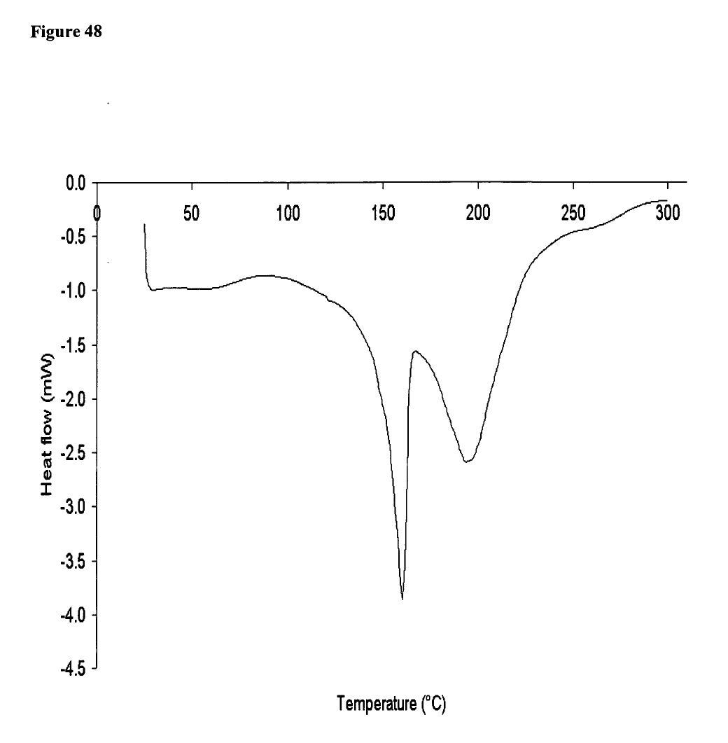

Form E was only formed in 1,2-propanediol with high cooling rates and it is a solvated form with 1,2-propanediol. A XRPD representative pattern is shown in FIG. 47.

3. (S)-Amlodipine-L-Malate Solvate (Form F)

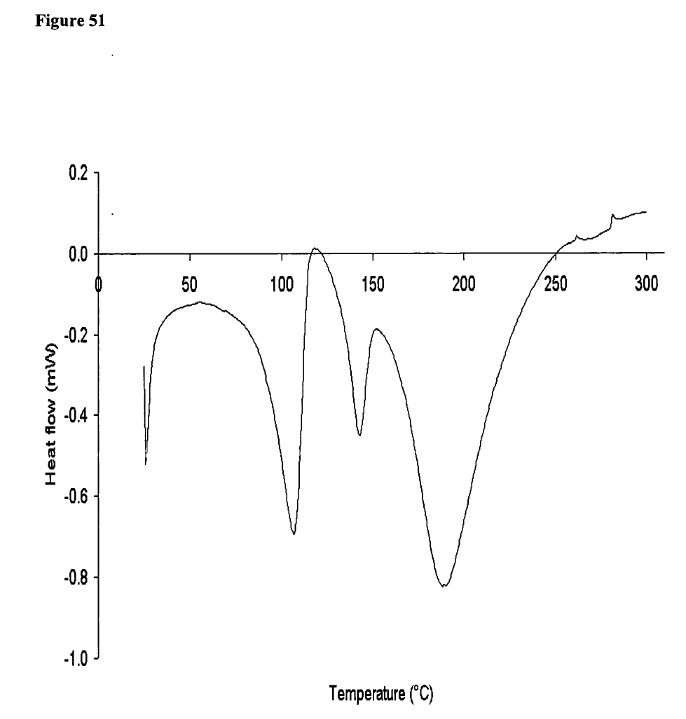

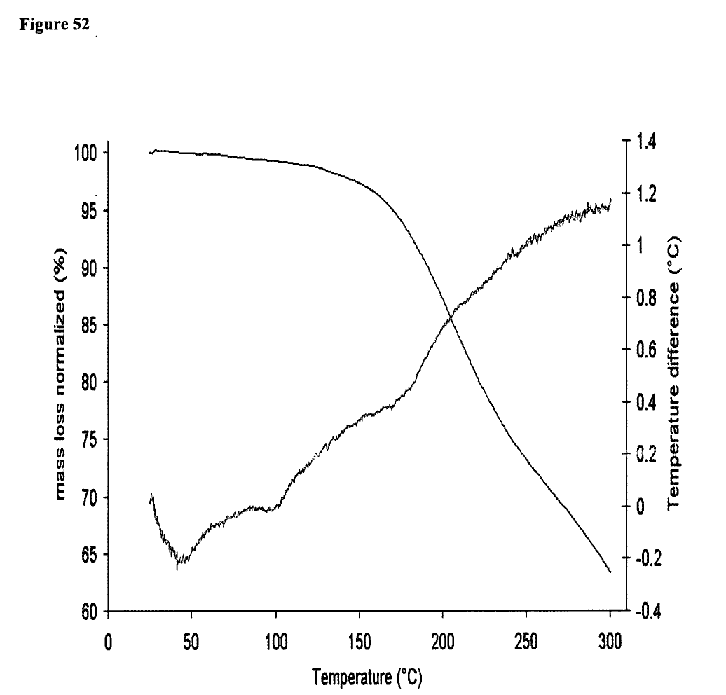

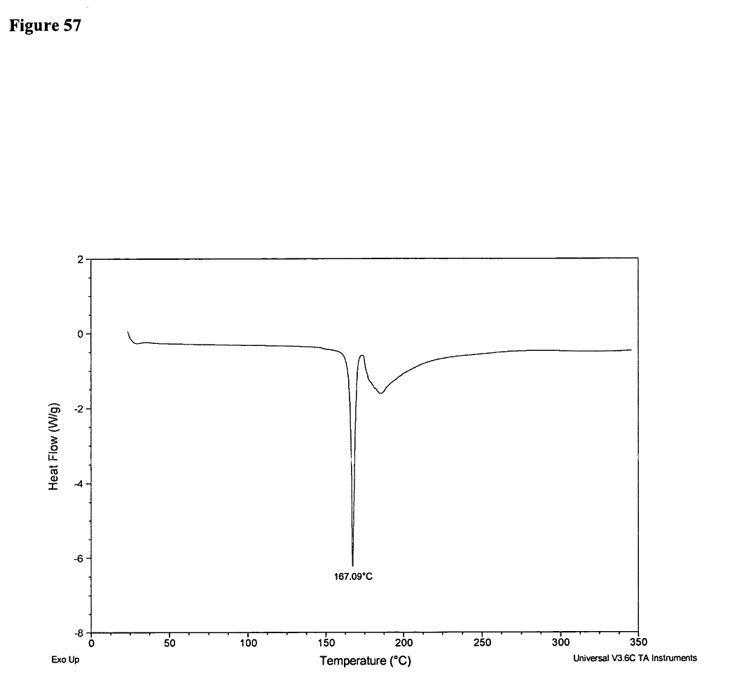

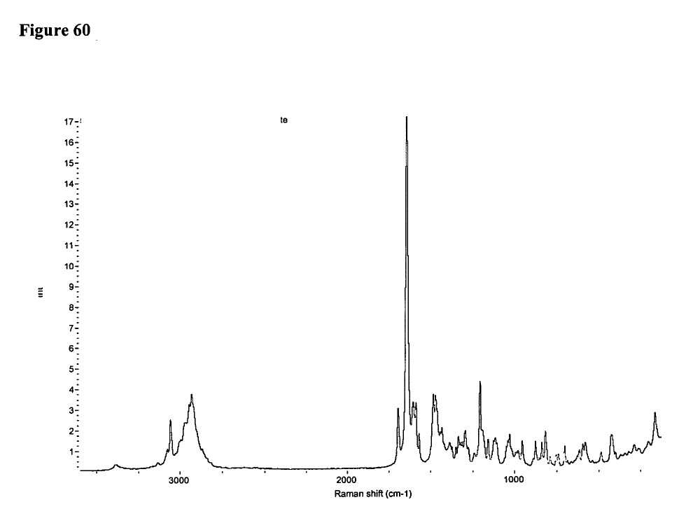

Form F was obtained as single phase and is strongly correlated with DMF as crystallization solvent, which indicate that it is a solvated form with DMF. The XRPD patterns of forms F and G are different, indicating that a different packing of the (S)-amlodipine molecules occurs in the two forms (FIGS. 57 and 60). It should be noticed that form F occurred in mixtures with form A also in other solvents, indicating that it is also a channel hydrate/solvate, but with a different crystal structure than form G. Based on the screening results we can conclude that form F can incorporate DMF, methanol and mixtures water:acetone (10:90), water:THF (80:20) and water:2-propanol (20:80). The TGA analysis of form F shows above 150° C. a high mass loss characteristic to a decomposition process occurred (FIG. 52). The DSC shows a melting endothermic peak at 106.6° C. after which it recrystallizes and melts at 149.3° C. (FIG. 51). A representative XRPD pattern is shown in FIG. 50.

3. (S)-Amlodipine-L-Malate Solvate (Form G)

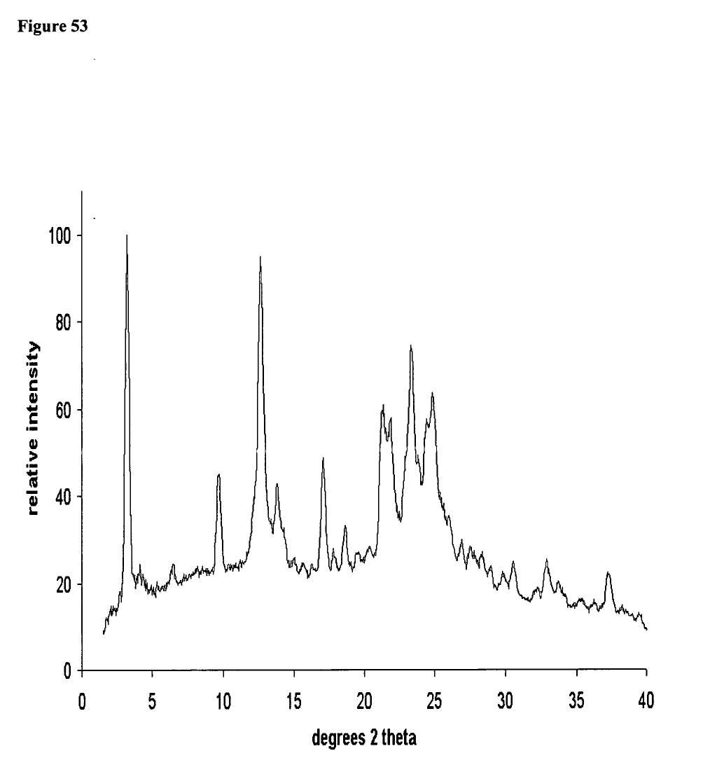

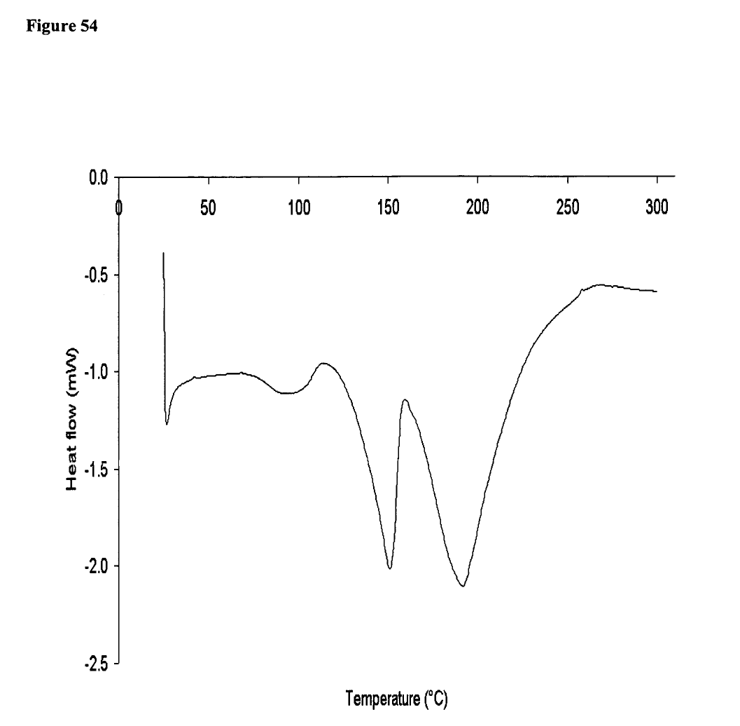

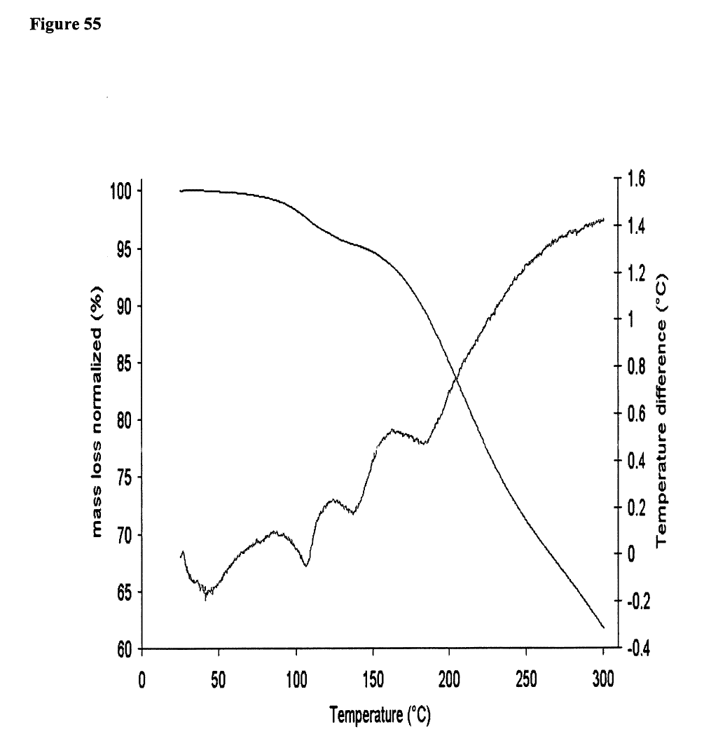

Form G is a pyridine solvate. The XRPD patterns of form G obtained in these different solvents are the same, indicating that different solvent molecules can be incorporated in certain cavities present in the crystal structure (structures called channel hydrates/solvates) without leading to modifications in the XRPD patterns. Based on the screening results we can conclude that form G is likely to be such a channel hydrate/solvate structure. Based on the screening results we can conclude that form G can incorporate pyridine, water and DMF:water (wet DMF). FIG. 53 presents the XRPD pattern of form G. The TGA analysis shows a 4.85% mass loss in the 91-125-° C. T interval after which a high mass loss characteristic to a decomposition process occurred (FIG. 55). The DSC shows a melting endothermic peak at 150.9° C. and a wide decomposition endothermic peak at 192.2° C. (FIG. 54).

### Example 15

### Characterization of (S)-Amlodipine-D-malate

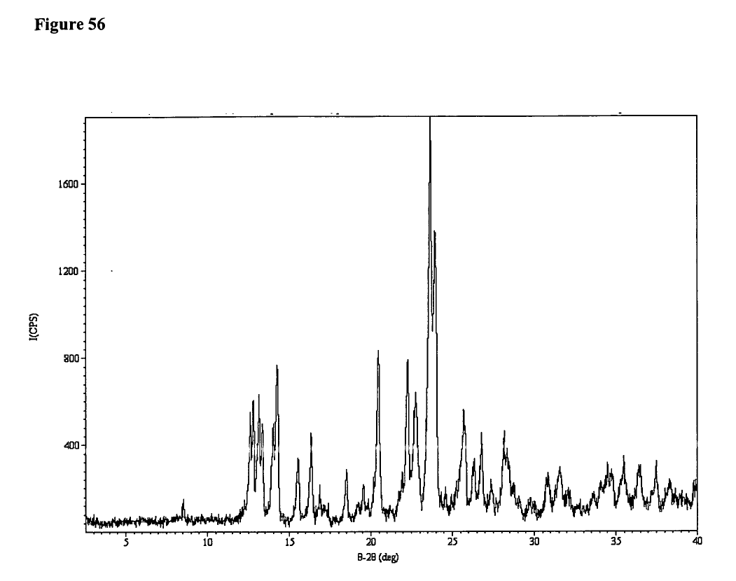

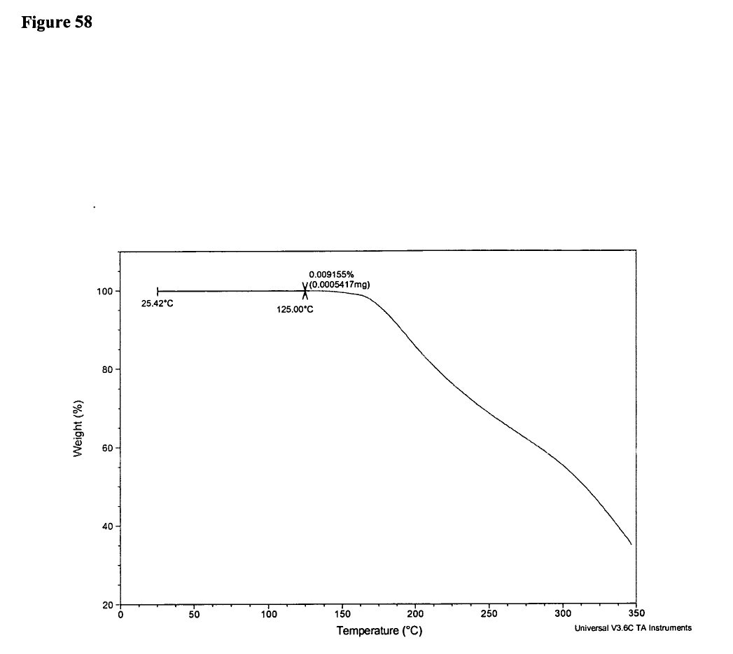

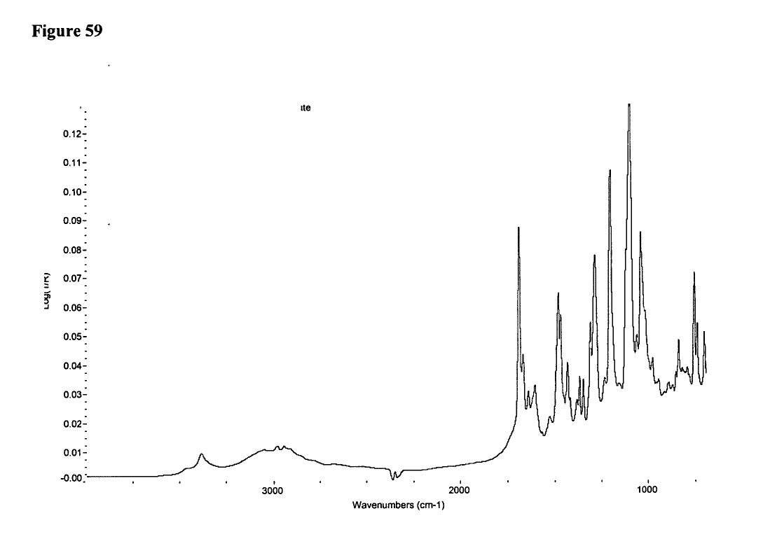

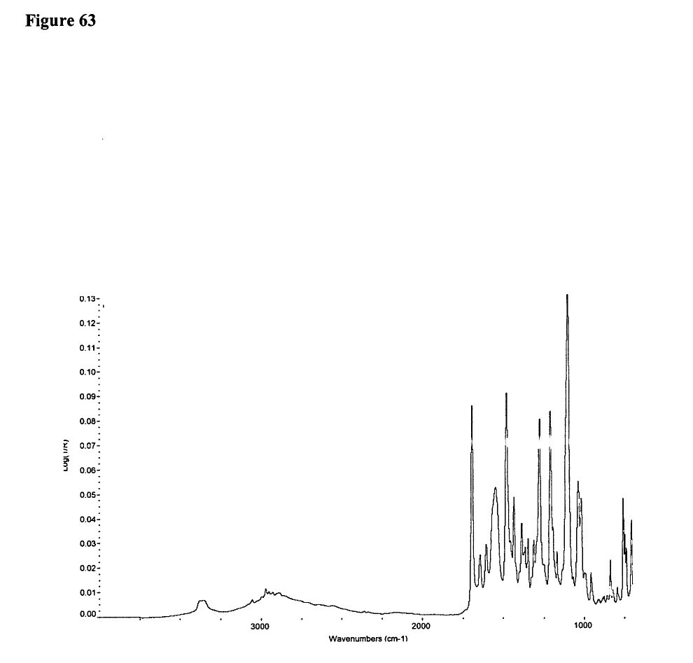

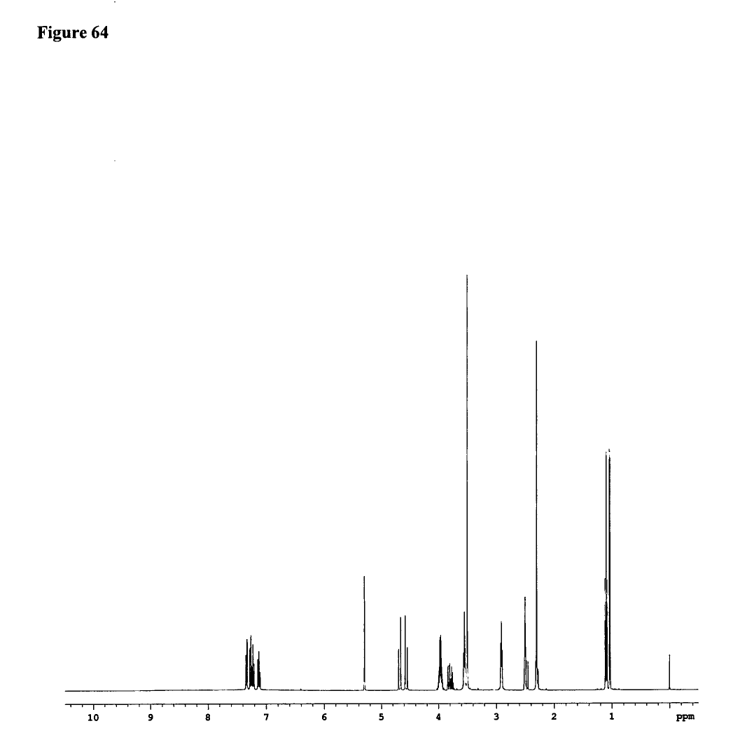

A representative XRPD pattern is shown in FIG. 56. The DSC curve (FIG. 57) shows an endotherm at 167° C. The TGA spectra (FIG. 58) shows less than 0.1% weight loss at 125° C. IR and Raman spectra are shown in FIGS. 63 and 64. Moisture balance experiments showed a 0.3% weight increase from 5% to 95% RH and a return to initial weight upon desorption. This is polymorph form D′.

### Example 16

### (R,S)-Amlodipine-(D,L)-Malate

1. Synthesis of (R,S)-Amlodipine-(D,L)-Malate

A 250 mL flask was charged with 6.45 g of racemic malic acid, 70 g of isopropanol, and 5.2 g of water. The mixture was heated to 52° C. and 19.5 g of racemic amlodipine in 15 g of methyl t-butyl ether was added. The mixture was stirred at 50° C. for 1 h, cooled to 0° C. over 1 h and stirred at 0° C. for 1 h. The slurry was filtered and the solid washed with 67 g of isopropanol. The solid was dried for 16 h at 55° C. under high vacuum to yield 21.2 g of amlodipine malate (0% ee) as a white solid.

2. General Procedure for Characterization

A. X-ray Powder Diffraction

X-ray powder diffraction (XRPD) analyses were performed using a Shimadzu XRD-6000 X-ray powder diffractometer using Cu Kα radiation. The instrument is equipped with a long fine focus X-ray tube. The tube voltage and amperage were set to 40 kV and 40 mA, respectively. The divergence and scattering slits were set at 1° and the receiving slit was set at 0.15 mm. Diffracted radiation was detected by a NaI scintillation detector. A θ-2θ continuous scan at 3′/min (0.4 sec/0.02° step) from 2.5 to 40°2θ was used. A silicon standard was analyzed to check the instrument alignment. Data were collected and analyzed using XRD-6000 v. 4.1. Samples were prepared for analysis by placing them in a silicon sample holder.

B. IR Spectroscopy

The IR spectra were acquired on a Magna-IR 860® Fourier transform infrared (FT-IR) spectrophotometer (Thermo Nicolet) equipped with an Ever-Glo mid/far IR source, an extended range potassium bromide (KBr) beamsplitter, and a deuterated triglycine sulfate (DTGS) detector. An attenuated total reflectance (ATR) accessory (the Thunderdome™, ThermoSpectra-Tech), with a germanium (Ge) crystal was used for data acquisition. Each spectrum represents 256 co-added scans collected at a spectral resolution of 4 cm⁻¹. A background data set was acquired with air. A Log 1/R(R=reflectance) spectrum was acquired by taking a ratio of these two data sets against each other. Wavelength calibration was performed using polystyrene.

C. Differential Scanning Calorimetry

Differential scanning calorimetry (DSC) was performed using a TA Instruments differential scanning calorimeter 2920. The sample was placed into an aluminum DSC pan, and the weight accurately recorded. The pan was covered with a lid and then crimped. The sample cell was equilibrated at 25° C. and heated under a nitrogen purge at a rate of 10° C./min, up to a final temperature of 350° C. Indium metal was used as the calibration standard. Reported temperatures are at the transition maxima.

D. Solution ¹HNMR Spectroscopy

The solution ¹H NMR spectra were acquired by Spectral Data Services of Champaign, Ill. at 25° C. with a Varian ^{UNITY}INOVA-400 spectrometer at a ¹H Larmor frequency of 399.798 MHz. The samples were dissolved in DMSO-d6. The spectra were acquired with a ¹H pulse width of 7 μs, a 2.5 second acquisition time, a 5 second delay between scans, a spectral width of 7000 Hz with 32768 data points, and 40 co-added scans. Each free induction decay (FID) was processed with 64K points and an exponential line broadening factor of 0.2 Hz to improve the signal-to-noise ratio. The residual peak from incompletely deuterated DMSO is at approximately 2.50 ppm

E. KF Analysis

Coulometric Karl Fischer (KF) analysis for water determination was performed using a Mettler Toledo DL39 Karl Fischer titrator. Approximately 10-20 mg of sample was placed in the KF titration vessel containing Hydranal-Coulomat AD and mixed for 60 seconds to ensure dissolution. The sample was then titrated by means of a generator electrode which produces iodine by electrochemical oxidation: 2 I−=>I₂+2e. Three or four replicates were obtained to ensure reproducibility.

3. Characterization of of (R,S)-Amlodipine-(D,L)-Malate

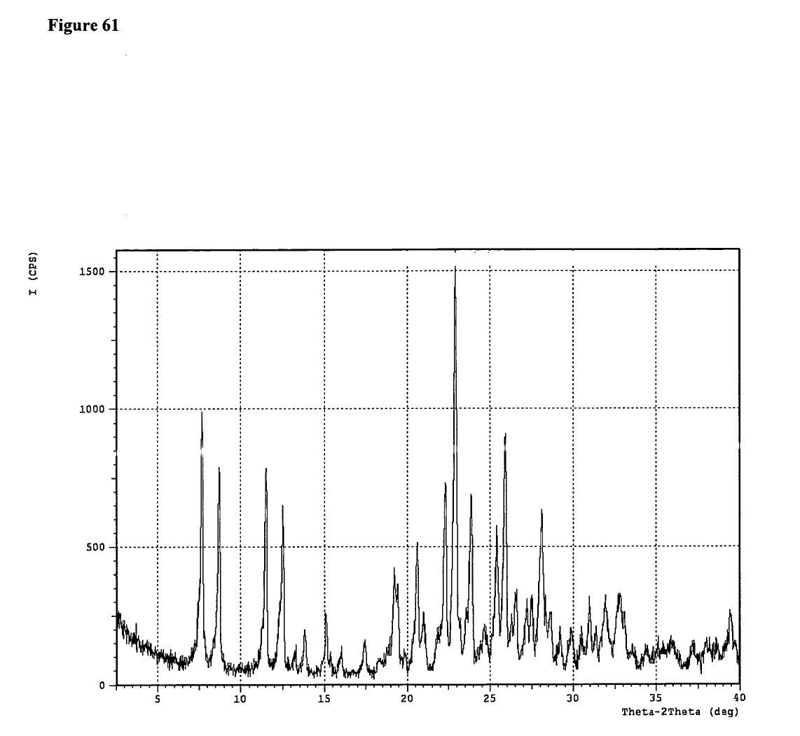

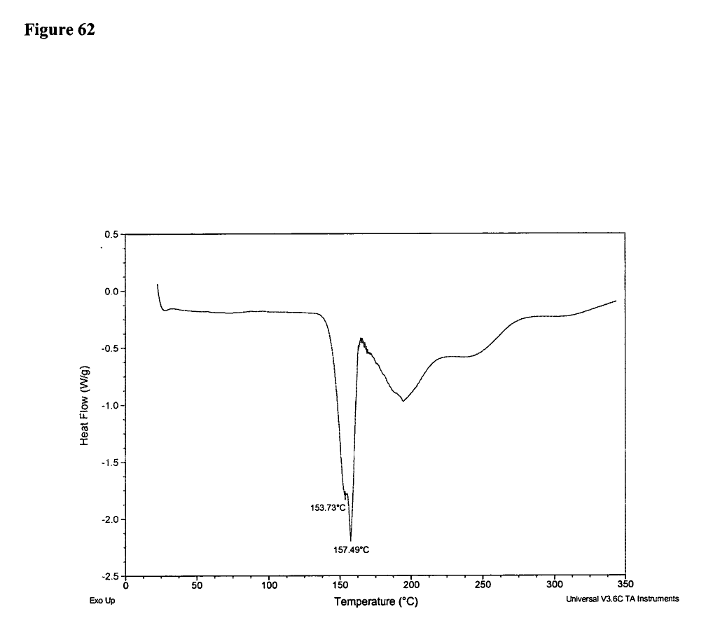

The (R,S)-amlodipine (D,L)-malate was analyzed by XRPD (FIG. 61) and DSC (FIG. 62). The sample was shown to be crystalline by XRPD, with an endothermic transition at 154° C. and 157° C. In addition to XRPD and DSC, the sample was analyzed by IR (FIG. 63), NMR (FIG. 64), and KF. Based on the NMR data, the sample is a hemi-malate salt. The NMR spectrum indicates that this sample contains isopropanol. Based on KF analysis the sample contained 0.19% water.

### Example 17

### (R,S)-Amlodipine L-Malate

1. Synthesis of (R,S)-Amlodipine-L-Malate

A 100 mL flask was charged with 2.34 g of racemic amlodipine, 21 mL of isopropanol, and 2.5 mL of methyl t-butyl ether. The mixture was heated to 52° C. and 0.78 g of L-malic acid in 2.6 mL of isopropanol and 0.7 mL of water was added. The resulting slurry was filtered and the solid washed with 1.3 mL of isopropanol. The solid was dried to yield 2.1 g of amlodipine L-malate (0% ee) as a white solid.

2. General Procedure for Characterization

A. X-Ray Powder Diffraction

XRPD analyses were performed using a Shimadzu XRD-6000 X-ray powder diffractometer using Cu Kα radiation. The instrument is equipped with a long fine focus X-ray tube. The tube voltage and amperage were set to 40 kV and 40 mA, respectively. The divergence and scattering slits were set at 1° and the receiving slit was set at 0.15 mm. Diffracted radiation was detected by a NaI scintillation detector. A theta-two theta continuous scan at 3′/min (0.4 sec/0.02° step) from 2.5 to 40° 2θ was used. A silicon standard was analyzed to check the instrument alignment. Data were collected and analyzed using XRD-6000 v. 4.1. Samples were prepared for analysis by placing them in a silicon sample holder.

B. Solution 1H NMR Spectroscopy

Solution ¹H NMR spectra were acquired by Spectral Data Services of Champaign, Ill. at 25° C. with a Varian UnityINOVA-400 spectrometer at a magnetic field strength of 9.39 Tesla (¹H Larmor frequency=399.798 MHz). The samples were dissolved in DMSO-d6. The spectra were acquired with a ¹H pulse width of 7.0 μs, a 2.50 second acquisition time, a 5 second delay between scans, a spectral width of 7000 Hz with 35K data points, and 40 co-added scans. The free induction decay (FID) was processed with 64K points and an exponential line broadening factor of 0.2 Hz to improve sensitivity. The residual peak from incompletely deuterated DMSO is at approximately 2.50 ppm.

C. Differential Scanning Calorimetry

Differential scanning calorimetry (DSC) was performed using a TA Instruments differential scanning calorimeter 2920. The sample was placed into an aluminum DSC pan, and the weight accurately recorded. The pan was covered with a lid, and then crimped. The sample cell was equilibrated at 25° C. and heated under a nitrogen purge at a rate of 10° C./min, up to a final temperature of 350° C. Indium metal was used as the calibration standard. Reported temperatures are at the transition maxima.

D. TGA Analysis

TGA analyses were performed using a TA Instruments 2950 thermogravimetric analyzer. Each sample was placed in an aluminum sample pan and inserted into the TGA furnace. The furnace was first equilibrated at 30° C., then heated under nitrogen at a rate of 10° C./min, up to a final temperature of 350° C. Nickel and Alumel™ were used as the calibration standards.

3. Characterization of of (R,S)-Amlodipine L-Malate

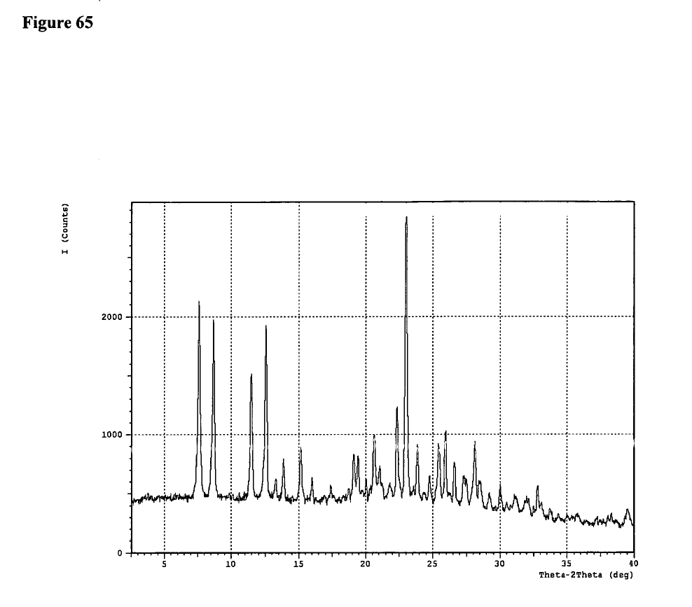

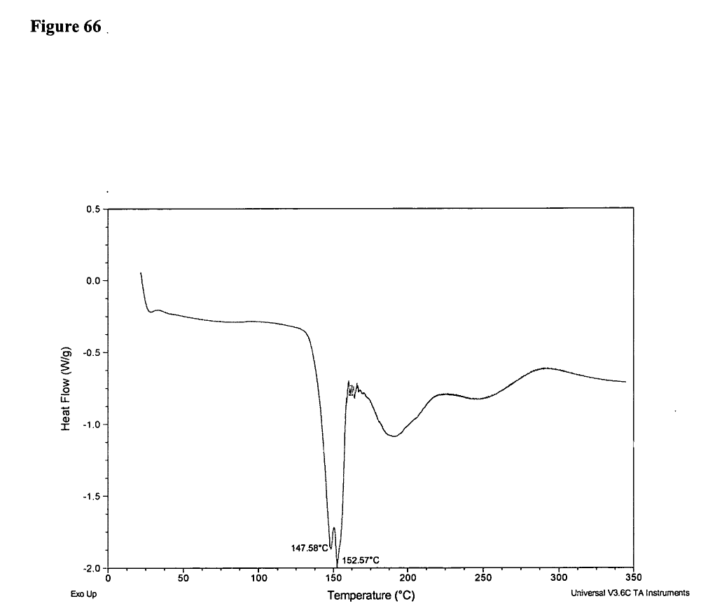

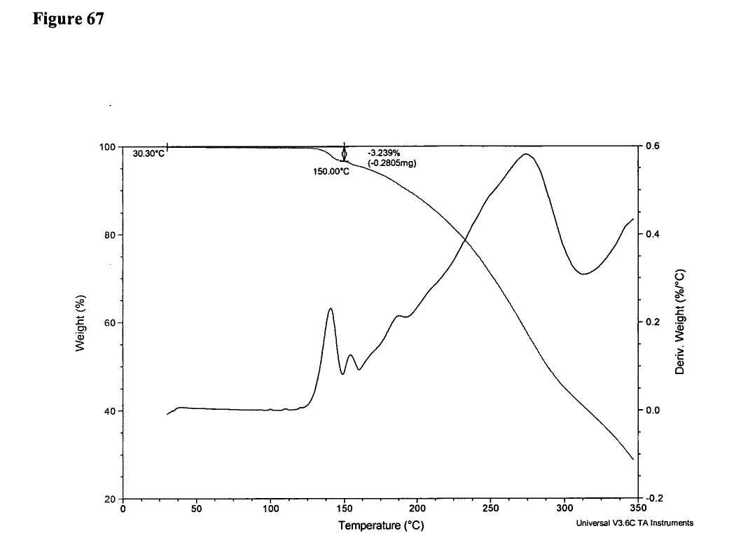

The (R,S)-amlodipine (L)-malate was analyzed by XRPD (FIG. 65) and DSC (FIG. 66). The sample was to have an endothermic transition at 148° C. and 153° C. In addition to XRPD and DSC, the sample was analyzed by TGA (FIG. 67). The TGA analysis shows a 3.2% mass loss at 150° C.

### INCORPORATION BY REFERENCE

All of the U.S. patents and U.S. patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. Optically pure (S)-amlodipine malate, or a polymorph, pseudopolymorph or solvate thereof.

2. The solvate according to claim 1, wherein said solvate comprises one or more solvent molecules independently selected from the group consisting of ketones, n-butanol, acetonitrile, benzonitrile, chloroform, cyclohexane, cyclopentanone, dibutylether, dichloromethane, N,N-dimethylformamide, dimethylsulfoxide, dioxane, ethanol, ethyl acetate, heptane, isopropanol, methanol, methyl ethyl ketone, N,N-dimethylacetamide, n-butylacetate, nitrobenzene, nitromethane, N-methylpyrrolidine, octane, propylene glycol, 1,2-propane diol, pyridine, tert-amylalcohol, tert-butyl methyl ether, tert-butyl methyl ether, tetrachloromethane, tetrahydrofuran, toluene, water, 2,2,2-trifluoroethanol and 2,2,4-trimethylpentane.

3. The solvate according to claim 1, wherein said solvate comprises one or more solvent molecules independently selected from the group consisting of water, dimethylsulfoxide, N-methylpyrroldinone, propylene glycol, pyridine, and N,N-dimethylformamide.

4. The compound of claim 1 or 2 or 3, wherein the enantiomeric excess of said optically pure (S)-amlodipine is at least about 90%.

5. The compound of claim 1 or 2 or 3, wherein the enantiomeric excess of said optically pure (S)-amlodipine is at least about 95%.

6. The compound of claim 1 or 2 or 3, wherein the enantiomeric excess of said optically pure (S)-amlodipine is at least about 99%.

7. The compound of claim 1 or 2 or 3, wherein said malate is optically pure L-malate.

8. The compound of claim 7, wherein the ratio of said optically pure (S)-amlodipine to said optically pure L-malate is about 1:1.

9. The compound of claim 7, wherein the ratio of said optically pure (S)-amlodipine to said optically pure L-malate is about 2:1.

10. The compound of claim 7, wherein the enantiomeric excess of said optically pure L-malate is at least about 90%.

11. The compound of claim 7, wherein the enantiomeric excess of said optically pure L-malate is at least about 95%.

12. The compound of claim 7, wherein the enantiomeric excess of said optically pure L-malate is at least about 99%.

13. Optically pure (S)-amlodipine L-malate, or a polymorph, pseudopolymorph or solvate thereof, wherein the optically pure (S)-amlodipine L-malate is substantially pure form A.

14. Optically pure (S)-amlodipine L-malate, or a polymorph, pseudopolymorph or solvate thereof, having an X-ray powder diffraction spectrum substantially the same as the X-ray powder diffraction spectrum of form A.

15. Optically pure (S)-amlodipine L-malate, or a polymorph, pseudopolymorph or solvate thereof, wherein the optically pure (S)-amlodipine L-malate is substantially pure form B.

16. Optically pure (S)-amlodipine L-malate, or a polymorph, pseudopolymorph or solvate thereof, having an X-ray powder diffraction spectrum substantially the same as the X-ray powder diffraction spectrum of form B.

17. Optically pure (S)-amlodipine L-malate, or a polymorph, pseudopolymorph or solvate thereof, wherein the optically pure (S)-amlodipine L-malate is substantially pure form C.

18. Optically pure (S)-amlodipine L-malate, or a polymorph, pseudopolymorph or solvate thereof, having an X-ray powder diffraction spectrum substantially the same as the X-ray powder diffraction spectrum of form C.

19. Optically pure (S)-amlodipine L-malate, or a polymorph, pseudopolymorph or solvate thereof, wherein the optically pure (S)-amlodipine L-malate is substantially pure form D.

20. Optically pure (S)-amlodipine L-malate, or a polymorph, pseudopolymorph or solvate thereof, having an X-ray powder diffraction spectrum substantially the same as the X-ray powder diffraction spectrum of form D.

21. Optically pure (S)-amlodipine L-malate, or a polymorph, pseudopolymorph or solvate thereof, wherein the optically pure (S)-amlodipine L-malate is substantially pure form E.

22. Optically pure (S)-amlodipine L-malate, or a polymorph, pseudopolymorph or solvate thereof, having an X-ray powder diffraction spectrum substantially the same as the X-ray powder diffraction spectrum of form E.

23. Optically pure (S)-amlodipine L-malate, or a polymorph, pseudopolymorph or solvate thereof, wherein the optically pure (S)-amlodipine L-malate is substantially pure form F.

24. Optically pure (S)-amlodipine L-malate, or a polymorph, pseudopolymorph or solvate thereof, having an X-ray powder diffraction spectrum substantially the same as the X-ray powder diffraction spectrum of form F.

25. Optically pure (S)-amlodipine L-malate, or a polymorph, pseudopolymorph or solvate thereof, wherein the optically pure (S)-amlodipine L-malate is substantially pure form G.

26. Optically pure (S)-amlodipine L-malate, or a polymorph, pseudopolymorph or solvate thereof, having an X-ray powder diffraction spectrum substantially the same as the X-ray powder diffraction spectrum of form G.

27. The compound of claim 1 or 2 or 3, wherein said malate is optically pure D-malate.

28. The compound of claim 27, wherein the ratio of said optically pure (S)-amlodipine to said optically pure D-malate is about 1:1.

29. The compound of claim 27, wherein the ratio of said optically pure (S)-amlodipine to said optically pure D-malate is about 2:1.

30. The compound of claim 27, wherein the enantiomeric excess of said optically pure D-malate is at least about 90%.

31. The compound of claim 27, wherein the enantiomeric excess of said optically pure D-malate is at least about 95%.

32. The compound of claim 27, wherein the enantiomeric excess of said optically pure D-malate is at least about 99%.

33. Optically pure (S)-amlodipine D-malate, or a polymorph, pseudopolymorph or solvate thereof, wherein the optically pure (S)-amlodipine D-malate is substantially pure form D′.

34. Optically pure (S)-amlodipine D-malate, or a polymorph, pseudopolymorph or solvate thereof, having an X-ray powder diffraction spectrum substantially the same as the X-ray powder diffraction spectrum of form D′.
